(19) United States
(12) Patent Application Publication
(10) Pub. No.: US 2002/0049176 A1
(43) Pub. Date: Apr. 25, 2002

(54) Modulation of mitochondrial mass and function for the treatment of diseases and for target and drug discovery

(76) Inventors: Christen M. Anderson, Encinitas, CA (US); William Clevenger, Oceanside, CA (US); K. David Becker, San Diego, CA (US); Kathryn Grako, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/795,651

(22) Filed: Feb. 27, 2001

Related U.S. Application Data
(63) Continuation-in-part of application No. 09/712,032, filed on Nov. 13, 2000.
(60) Provisional application No. 60/164,533, filed on Nov. 10, 1999.

(51) Int. Cl. A61K 48/00

(52) U.S. Cl. 514/44

(57) ABSTRACT

Compositions and methods are provided for the treatment of diseases associated with altered mitochondrial function, and in particular, for type 2 diabetes mellitus. Administration of an agent that increases mitochondrial mass, including promotion of mitochondrial biogenesis by induction of a PGC gene (e.g., PGC-1) and/or a nuclear regulatory factor gene (e.g., NRF-1), is also disclosed. Screening assays for agents that regulate such genes involved in mitochondrial biogenesis, and for genes and gene products that are regulated by such genes involved in mitochondrial biogenesis, are also provided.

### CROSS REFERENCES TO RELATED APPLICATIONS

This application is a continuation-in-part of U.S. Ser. No. 09/712,032, filed Nov. 13, 2000, which application claims the benefit of Provisional Application No. 60/164,533, filed Nov. 10, 1999, and which applications are incorporated herein by reference in their entirety.

### FIELD OF THE INVENTION

The invention relates generally to the modulation of mitochondrial mass and function for the treatment of diseases and for target and drug discovery.

### BACKGROUND OF THE INVENTION

The cell is the basic unit of life and comprises a variety of subcellular compartments including, e.g., organelles. An organelle is a structural component of a cell that is physically separated, typically by one or more membranes, from other cellular components, and which carries out specialized cellular functions.

Mitochondria contain their own DNA genome. These organellar genomes encode a fraction of the gene products required for organellar function, the remainder of such gene products being encoded by the nuclear genome. Relatively little is known about the mechanisms by which mitochondrial gene products, which may be encoded by nuclear sequences or sequences found in the organellar genomes, are coordinately regulated (Surpin and Chory, Essays Biochem. 32:113-125, 1997).

The organelle known as the mitochondrion (plural, mitochondria) is the main energy source in cells of higher organisms. Mitochondria provide direct and indirect biochemical regulation of a wide array of cellular respiratory, oxidative and metabolic processes. These include electron transport chain (ETC) activity, which drives oxidative phosphorylation to produce metabolic energy in the form of adenosine triphosphate (ATP), and which also underlies a central mitochondrial role in intracellular calcium homeostasis.

In addition to their role in energy production in growing cells, mitochondria (or, at least, mitochondrial components) participate in programmed cell death (PCD), also known as apoptosis (Newmeyer et al., Cell 79:353-364, 1994; Liu et al., Cell 86:147-157, 1996). Apoptosis is apparently required for normal development of the nervous system, and for proper functioning of the immune system. Moreover, some disease states are thought to be associated with either insufficient or excessive levels of apoptosis (e.g., cancer and autoimmune diseases in the first instance, and stroke damage and neurodegeneration in Alzheimer's disease in the latter case). For general reviews of apoptosis, and the role of mitochondria therein, see, e.g., Green and Reed (Science 281:1309-1312, 1998), Green (Cell 94:695-698, 1998) and Kromer (Nature Medicine 3:614-620, 1997). Altered or defective mitochondrial activity, including but not limited to failure at any step of the ETC, may result in the generation of highly reactive free radicals that have the potential of damaging cells and tissues. These free radicals may include reactive oxygen species (ROS) such as superoxide, peroxynitrite and hydroxyl radicals, and potentially other reactive species that may be toxic to cells. For example, oxygen free radical induced lipid peroxidation is a well established pathogenetic mechanism in central nervous system (CNS) injury such as that found in a number of degenerative diseases, and in ischemia (i.e., stroke).

In addition to free radical mediated tissue damage, there are at least two deleterious consequences of exposure to reactive free radicals arising from mitochondrial dysfunction that adversely impact the mitochondria themselves. First, free radical mediated damage may inactivate one or more of the myriad proteins of the ETC. Second, free radical mediated damage may result in catastrophic mitochondrial collapse that has been termed “permeability transition” (PT) or “mitochondrial permeability transition” (MPT). According to generally accepted theories of mitochondrial function, proper ETC respiratory activity requires maintenance of an electrochemical potential (Δψm) in the inner mitochondrial membrane by a coupled chemiosmotic mechanism, as described herein. Free radical oxidative activity may dissipate this membrane potential, thereby preventing ATP biosynthesis and halting the production of a vital biochemical energy source. In addition, mitochondrial proteins such as cytochrome c and “apoptosis inducing factor” may leak out of the mitochondria after permeability transition and may induce the genetically programmed cell suicide sequence known as apoptosis or programmed cell death (PCD). Therefore, mere determination of free radical induced damage, such as lipid peroxidation, is not an accurate or early indicator of mitochondrial dysfunction.

Altered mitochondrial function characteristic of the mitochondria associated diseases may also be related to loss of mitochondrial membrane electrochemical potential by mechanisms other than free radical oxidation, and permeability transition may result from direct or indirect effects of mitochondrial genes, gene products or related downstream mediator molecules and/or extramitochondrial genes, gene products or related downstream mediators, or from other known or unknown causes. Loss of mitochondrial potential therefore may be a critical event in the progression of diseases associated with altered mitochondrial function, including degenerative diseases.

Mitochondrial defects, which may include defects related to the discrete mitochondrial genome that resides in mitochondrial DNA and/or to the extramitochondrial genome, which includes nuclear chromosomal DNA and other extramitochondrial DNA, may contribute significantly to the pathogenesis of diseases associated with altered mitochondrial function. For example, alterations in the structural and/or functional properties of mitochondrial components comprising subunits encoded directly or indirectly by mitochondrial and/or extramitochondrial DNA, including alterations deriving from genetic and/or environmental factors or alterations derived from cellular compensatory mechanisms, may play a role in the pathogenesis of any disease associated with altered mitochondrial function. A number of degenerative, hyperproliferative and other types of diseases are thought to be caused by, or to be associated with, alterations (e.g., statistically significant increases or decreases) in mitochondrial ftunction. These include, for example, Alzheimer's Disease, Parkinson's Disease, Huntington's disease, diabetes mellitus, and hyperproliferative disorders, such as cancer, tumors and psoriasis. Additional diseases with which altered mitochondrial function or mitochondrial dysfunction has been associated include amyotrophic lateral sclerosis (ALS), Friedreich's ataxia, colon cancer, and exercise intolerance. Exercise intolerance has been associated with mutations in the mitochondrial gene encoding cytochrome b (Andreu et al., New Engl. J Med. 341:1037-1044, 1999), while colon cancer has been linked to a series of mtDNA mutations (Polyak et al., Nat Genet 20:3 291-3; 1998). Familial ALS, which is caused by mutations in Cu/Zn superoxide dismutase, is also associated with abnormalities of the mitochondrial electron transfer chain that include increased complex I and complex II-III activities (Browne et al., J. Neurochem. 71:281-287, 1998). The relationship between Friedreich's ataxia and mitochondrial function is less direct. Frataxin, a protein implicated in the ataxia, is homologous to a yeast mitochondrial gene which, when disrupted, causes accumulation of iron in mitochondria and disruption of mtDNA. The extensive list of mitochondria associated diseases, i.e., diseases associated with altered mitochondrial function and/or mitochondrial mutations, continues to expand as aberrant mitochondrial or mitonuclear activities are implicated in particular disease processes.

Mitochondrial ultrastructural characterization reveals the presence of an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix. (For a review, see, e.g., Ernster and Schatz, J. Cell Biol. 91:227s-255s, 1981.) The cristae, originally postulated to occur as infoldings of the inner mitochondrial membrane, have recently been characterized using three-dimensional electron tomography as also including tube-like conduits that may form networks, and that can be connected to the inner membrane by open, circular (30nm diameter) junctions (Perkins et al, Journal of Structural Biology 119:260-272, 1997). While the outer membrane is freely permeable to ionic and non-ionic solutes having molecular weights less than about ten kilodaltons, the inner mitochondrial membrane exhibits selective and regulated permeability for many small molecules, including certain cations, and is impermeable to large (>˜10 kDa) molecules.

Four of the five multisubunit protein complexes (Complexes 1, III, IV and V) that mediate ETC activity are localized to the inner mitochondrial membrane. The remaining ETC complex (Complex II) is situated in the matrix. In at least three distinct chemical reactions known to take place within the ETC, protons are moved from the mitochondrial matrix, across the inner membrane, to the intermembrane space. This disequilibrium of charged species creates an electrochemical potential of approximately 220 mV referred to as the “protonmotive force” (PMF). PMF, which is often represented by the notation Δp, corresponds to the sum of the electric potential (ATm) and the pH differential (ΔpH) across the inner mitochondrial membrane according to the equation

Δp=Δψ−ZΔpH,

wherein Z stands for −2.303 RT/F. The value of Z is −59 at 25° C. when Δp and Δψm are expressed in mV and ΔpH is expressed in pH units (see, e.g., Ernster et al., 1981 J. Cell Biol. 91:227s-255s and references cited therein).

Many mitochondrial finctions depend in part or entirely on Δψm. For example, Δψm provides the energy for phosphorylation of adenosine diphosphate (ADP) to yield ATP by ETC Complex V, a process that is coupled stoichiometrically with transport of a proton into the matrix. Furthermore, Δψm is also the driving force for the influx of cytosolic Ca²⁺ into the mitochondrion. Even fundamental biological processes, such as translation of mRNA molecules to produce polypeptides, may be dependent on Δψm (Cote et al., J. Biol. Chem. 265:7532-7538, 1990).

Under normal metabolic conditions, the inner membrane is impermeable to proton movement from the intermembrane space into the matrix, leaving ETC Complex V as the sole means whereby protons can return to the matrix. When, however, the integrity of the inner mitochondrial membrane is compromised, as occurs during mitochondrial permeability transition (MPT) that accompanies certain diseases associated with altered mitochondrial function, protons are able to bypass the conduit of Complex V without generating ATP, thereby uncoupling respiration. During MPT, Δψm collapses and mitochondrial membranes lose the ability to selectively regulate permeability to solutes both small (e.g., ionic Ca²⁺, Na⁺, K⁺, H⁺) and large (e.g., proteins).

Non-insulin-dependent diabetes mellitus (NIDDM, type II diabetes) is characterized by abnormalities in insulin secretion and insulin action. Subjects having NIDDM constitute 90-95% of the approximately 6 million diagnosed diabetics in the United States. NIDDM is characterized by hyperglycemia, the result of insulin resistance in peripheral tissues (e.g., skeletal muscle and adipose tissue) where insulin-stimulated uptake/utilization of glucose is blunted, and in liver, where insulin suppression of glucose output is insufficient. Such impairment of insulin action plays an important role in the development of glucose intolerance and elevated fasting levels of blood glucose. Careful attention to diet and exercise comprise a first-line therapy for NIDDM patients, who may also take hypoglycemic drugs to control blood glucose levels. The most widely used hypoglycemic agents are various formulations of insulin and sulfonylureas. A major drawback with these therapies is the occurrence of potentially life-threatening hypoglycemia due to hyperinsulinemia.

Type 2 diabetes mellitus, or “late onset” diabetes, is a common, degenerative disease affecting 5 to 10 percent of the population in developed countries. The propensity for developing type 2 diabetes mellitus (“type 2 DM”) is reportedly maternally inherited, suggesting a mitochondrial genetic involvement (Alcolado et al., Br. Med. J. 302:1178-1180, 1991; Reny, International J. Epidem. 23:886-890, 1994). Diabetes is a heterogeneous disorder with a strong genetic component; monozygotic twins are highly concordant and there is a high incidence of the disease among first degree relatives of affected individuals.

Current pharmacological therapies for type 2 DM include injected insulin, and oral agents that are designed to lower blood glucose levels. Currently available oral agents include (i) the sulfonylureas, which act by enhancing the sensitivity of the pancreatic beta cell to glucose, thereby increasing insulin secretion in response to a given glucose load; (ii) the biguanides, which improve glucose disposal rates and inhibit hepatic glucose output; (iii) the thiazolidinediones, which improve peripheral insulin sensitivity through interaction with nuclear peroxisome proliferator-activated receptors (PPAR, see, e.g., Spiegelman, 1998 Diabetes 47:507-514; Schoonjans et al., 1997 Curr. Opin. Lipidol. 8:159-166; Staels et al., 1997 Biochimie 79:95-99), (iv) repaglinide, which enhances insulin secretion through interaction with ATP-dependent potassium channels; and (v) acarbose, which decreases intestinal absorption of carbohydrates.

At the cellular level, the degenerative phenotype that may be characteristic of late onset diabetes mellitus includes indicators of mitochondrial respiratory function, for example impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, circulating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies, peripheral and sensory neuropathies and blindness.

It is clear that none of the current pharmacological therapies corrects the underlying biochemical defect in type 2 DM. Neither do any of these currently available treatments improve all of the physiological abnormalities in type 2 DM such as impaired insulin secretion, insulin resistance and/or excessive hepatic glucose output. In addition, treatment failures are common with these agents, such that multi-drug therapy is frequently necessary.

Due to the strong genetic component of diabetes mellitus, the nuclear genome has been the main focus of the search for causative genetic mutations. However, despite intense effort, nuclear genes that segregate with diabetes mellitus are rare and include, for example, mutations in the insulin gene, the insulin receptor gene and the glucokinase gene. By comparison, although a number of mitochondrial genes that segregate with diabetes mellitus have been reported (see generally, e.g., PCT/US95/04063), relationships amongst mitochondrial and extramitochondrial factors that contribute to cellular respiratory and/or metabolic activities as they pertain to diabetes remain poorly understood.

Cellular demands for increased energy supply are often accompanied by an increase in respiratory activity, which can include an increase in mitochondrial mass. Mitochondrial proliferation can be induced by a variety of environmental stimuli, such as exercise; induction of mitoproliferation has also been observed following direct electrical stimulation of cultured cardiomyocytes. Such increases in mitochondrial mass require exquisite coordination of specific nuclear and mitochondrial genes and factors involved in mitochondrial biogenesis.

As noted above, the majority of gene products required for mitochondrial respiratory function are encoded in the nuclear genome. One approach to understanding nucleo-mitochondrial (or mitonuclear) interactions in mammalian cells has been the identification of nuclear transcription factors that regulate the expression of such gene products. For example, using this approach two transcription factors known as nuclear respiratory factors-1 and -2 (NRF-1 and NRF-2) have been purified, and nucleic acid sequences encoding NRF-1 and NRF-2 have been molecularly cloned. The DNA binding and transcriptional specificities of these proteins have implicated them in the expression of many respiratory subunits along with key components of, inter alia, mitochondrial transcription, replication and heme biosynthetic mechanisms.

Nuclear respiratory factor 1 (NRF-1) is thus believed to comprise a transcription factor occurring as a homodimer of a 54 Kd polypeptide encoded by the nuclear gene nrf-1 (Evans and Scarpulla, Genes & Development 4:1023-1034 (1990), Scarpulla, J. Bioenergetics and Biomembranes 29:109-119 (1997), Moyes et al., J. Exper. Biol. 201:299-307 (1998)). NRF-1 binds to the upstream promoters of nuclear genes that encode respiratory components associated with mitochondrial transcription and replication; accordingly NRF-1 binding sites are found in many genes that encode respiratory proteins. A second transcription factor, NRF-2, is linked to cytochrome c oxidase subunit IV and Vb promoter function. NRF-1 and NRF-2 act on an overlapping subset of nuclear genes required for mitochondrial respiratory activity.

Peroxisome proliferator-activated receptor gamma (PPAR-gamma) is a member of the steroid/thyroid/retinoid superfamily of ligand-activated transcription factors. PPAR-gamma is one member of a subfamily of closely-related PPARs encoded by independent genes. Three mammalian PPARs have been presumptively identified and termed PPAR-alpha, PPAR-gamma, and NUC-1. PPARs regulate expression of target genes by binding to DNA sequence elements, termed PPAR response elements (PPRE), as heterodimers with the retinoid X receptors. A second isoform of PPAR-gamma, termed PPAR-gamma2, has been presumptively identified from a mouse adipocyte library.

The PPAR-gamma coactivator (PGC-1) gene is encoded by the nuclear genome and is a transcriptional coactivator of several known nuclear receptors, including PPAR-gamma (Butow et al., Current Biology 9:R767-R769, 1999; Puigserver et al., Cell 92:829-839, 1998; Lowell, Current Biology 8:R517-R520, 1998; Freake, Nutrition Reviews 57:154-156, 1999; Chawla et al. Endocrinol. 135:798-800, 1994). PGC-1 also is believed to stimulate induction of the gene expression of the nuclear factors NRF-1 and NRF-2. In response to external stimuli, PGC-1 is believed to direct the expression of key regulatory molecules responsible for the synthesis of nuclear-encoded components of the oxidative phosphorylation apparatus, including, for example (a) a mitochondrial transcription factor, mtTRFA, that is believed to control the replication and transcription of the mitochondrial genome; and (b) a unique family of uncoupling proteins, the UCP's, that uncouple mitochondrial electron transport from ATP synthesis.

For example, according to non-limiting theory, in a biologically relevant regulatory pathway, the nuclear pgc-1 gene is transcribed in response to an appropriate stimulus (e.g., cold) and the transcript subsequently translated. The expressed PGC-1 protein then binds to regulatory elements that modulate the expression of a variety of genes, including nrf-1, which results in the expression of NRF-1. NRF-1 can then bind to PGC-1 to form a NRF-1:PGC-1 complex, which is itself a transcription factor able to regulate the transcription of mtTRFA. Regulation (e.g., modulation) of mitochondrial DNA replication and/or transcription may be then mediated by mtTRFA.

From the foregoing, it is apparent that there exists a need to further understand the role of mitochondria, mitochondrial activity and mitochondrial biogenesis in a variety of diseases, including diabetes (e.g., types 1 and 2 DM), and further to identify molecular components such as gene products, naturally occurring agents and non-natural agents that regulate such mitochondrial processes. The present invention provides these and other related advantages, as will become more apparent from the detailed description of the invention provided herein.

### SUMMARY OF THE INVENTION

The present invention relates to compositions and methods for the treatment of a disease associated with altered mitochondrial function, for identification of useful molecular targets and for drug discovery, including compositions and methods for increasing mitochondrial mass and mitochondrial function. The present invention thus provides therapeutic compositions (and related agents) and methods for altering (e.g., increasing in a statistically significant manner relative to untreated controls) mitochondrial mass and/or altering (i.e., improving by increasing or decreasing in a statistically significant manner relative to untreated controls) mitochondrial function in cells of an animal or human subject which has, or is suspected of being prone to developing, diabetes.

In one aspect the invention provides a method for treating a disease associated with altered mitochondrial function comprising administering an agent that increases mitochondrial mass in cells in an individual in need thereof. In some emgodiments the disease associated with altered mitochondrial function is diabetes, which in certain further embodiments is type 2 diabetes mellitus. In another embodiment the agent that increases mitochondrial mass induces expression of a gene that is a PGC gene or a NRF gene. In certain further embodiments the PGC gene is PGC-1, or the NRF gene is NRF-1. In certain other further embodiments the agent that increases mitochondrial mass is provided in a pharmaceutical composition. In another embodiment the invention provides a method for treating a disease associated with altered mitochondrial function comprising administering an agent that alters mitochondrial function in cells in an individual in need thereof. In certain embodiments the disease associated with altered mitochondrial function is diabetes, which in certain further embodiments is type 2 diabetes mellitus. In other embodiments the mitochondrial function is oxygen consumption, mitochondrial biogenesis, oxidative phosphorylation, glucose-stimulated insulin secretion or apoptosis. In certain embodiments the cells are pancreatic cells, which in certain further embodiments are pancreatic beta cells. In other embodiments the cells are treated with at least one agent that is an agent that alters the expression of a PGC gene, an agent that alters the expression of a NRF gene, an agent that alters the activity of a PGC gene product or an agent that alters the activity of a NRF gene product. In certain further embodiments the agent is a polypeptide, a nucleic acid, a small molecule, a gene therapy construct or a test compound. In certain other further embodiments the agent that increases mitochondrial mass or that alters mitochondrial function is provided in a pharmaceutical composition. In certain further embodiments the PGC gene is PGC- 1, or the NRF gene is NRF- 1.

In another embodiment the present invention provides a method for identifying an agent for treating a disease associated with altered mitochondrial function, comprising contacting a cell comprising a regulatory expression construct with at least one candidate agent, wherein the regulatory expression construct comprises at least one regulatory element that is derived from a gene that is a PGC gene or a NRF gene and that is operably linked to a reporter gene, and wherein the candidate agent alters the expression of the reporter gene relative to reporter gene expression in the absence of the candidate agent, and therefrom identifying an agent for treating the disease associated with altered mitochondrial funtion. In another embodiment the invention provides a method for identifying an agent for treating a disease associated with altered mitochondrial function, comprising contacting a candidate agent with a sample comprising a mitochondrion, wherein the mitochondrion comprises an expression construct encoding one or more proteins selected from an NRF protein or a PGC protein; and determining a level of at least one indicator of mitochondrial function, wherein the candidate agent alters the level of the indicator of mitochondrial flnction relative to the level of said indicator in the absence of the agent, and therefrom identifying an agent for treating a disease associated with altered mitochondrial function.

In another embodiment the invention provides a method for identifying an agent for treating a disease associated with altered mitochondrial function, comprising contacting a candidate agent with a sample comprising a mitochondrion, wherein the mitochondrion comprises a product of an expression construct encoding one or more proteins selected from an NRF protein or a PGC protein; and determining a level of at least one indicator of mitochondrial function, wherein the candidate agent alters the level of the indicator of mitochondrial function relative to the level of the indicator in the absence of the agent, and therefrom identifying an agent for treating a disease associated with altered mitochondrial function. In another embodiment the invention provides a method for identifying an agent for treating a disease associated with altered mitochondrial function, comprising contacting a candidate agent with a sample comprising a cell containing a mitochondrion, wherein the cell comprises an expression construct encoding one or more proteins selected from an NRF protein or a PGC protein; and determining a level of at least one indicator of mitochondrial function, wherein the candidate agent alters the level of the indicator of mitochondrial function relative to the level of said indicator in the absence of the agent, and therefrom identifying an agent for treating a disease associated with altered mitochondrial function.

In another embodiment the invention provides a method for identifying an agent for treating a disease associated with altered mitochondrial function, comprising contacting a candidate agent with a sample comprising a cell containing a mitochondrion, wherein the cell comprises a product of an expression construct encoding one or more proteins selected from an NRF protein or a PGC protein; and determining a level of at least one indicator of mitochondrial function, wherein the candidate agent alters the level of the indicator of mitochondrial function relative to the level of said indicator in the absence of the agent, and therefrom identifying an agent for treating a disease associated with altered mitochondrial function. In certain further embodiments of the above described methods, the disease associated with altered mitochondrial function is diabetes. In certain further embodiments of the above described methods, the indicator of mitochondrial function is glucose responsiveness.

In another embodiment the invention provides a method for identifying a regulator of mitochondrial biogenesis, comprising contacting a stimulus with a cell comprising a mitochondrion under conditions and for a time sufficient to induce mitochondrial biogenesis; and detecting an altered level of a candidate signaling molecule, wherein an altered level of the candidate signaling molecule in a cell that has been contacted with the stimulus that induces mitochondrial biogenesis relative to the level of the candidate signaling molecule in a cell that has not been contacted with the stimulus indicates that the candidate signaling molecule is a regulator of mitochondrial biogenesis. In a further embodiment the stimulus is selected cold stress, an electrical stimulus or an adrenergic stimulus. In certain other embodiments mitochondrial biogenesis is detected by determining an indicator of mitochondrial function that is oxygen consumption, amount of mitochondrial DNA, mitochondrial mass or an ATP biosynthesis factor. In certain other embodiments the candidate signaling molecule regulates activity of a gene that is a PGC gene or a NRF gene. In certain other embodiments the candidate signaling molecule is regulated by a gene that is a PGC gene or a NRF gene. In certain other embodiments the altered level of the candidate signaling molecule is a level of a nucleic acid, a level of a polypeptide and a level of phosphorylation of a protein.

In another embodiment the invention provides a method for identifying an agent that alters activity of a regulator of mitochondrial biogenesis for treating a disease associated with altered mitochondrial function, comprising contacting, in the presence of a candidate agent, a stimulus with a cell comprising a mitochondrion under conditions and for a time sufficient to induce an altered level of a signaling molecule that regulates mitochondrial biogenesis, wherein an altered level of the signaling molecule that regulates mitochondrial biogenesis in a cell that has been contacted with the candidate agent relative to the level of the signaling molecule that regulates mitochondrial biogenesis in a cell that has not been contacted with the candidate agent indicates that the agent alters activity of a regulator of mitochondrial biogenesis.

In another embodiment the invention provides a method of identifying a gene encoding a target for therapeutic intervention in a disease associated with altered mitochondrial finction, comprising: (a) comparing (i) a first plurality of isolated nucleic acid molecules derived from a first biological source in which expression of a gene known to alter mitochondrial biogenesis has been induced, to (ii) a second plurality of isolated nucleic acid molecules derived from a second biological source in which expression of the gene known to alter mitochondrial biogenesis has not been induced, wherein the presence of at least one differentially expressed nucleic acid molecule in (i) or (ii) indicates the differentially expressed nucleic acid molecule is a candidate gene encoding a target for therapeutic intervention in a disease associated with altered mitochondrial function; and (b) determining that altered expression of said candidate gene alters mitochondrial biogenesis, and therefrom identifying a gene encoding a target for therapeutic intervention in a disease associated with altered mitochondrial function. In certain further embodiments the gene known to alter mitochondrial biogenesis is a PGC gene or a NRF gene. In certain other embodiments mitochondrial biogenesis is determined by measuring oxygen consumption. In another embodiment mitochondrial biogenesis is determined by detecting an indicator of mitochondrial function that is oxygen consumption, amount of mitochondrial DNA, mitochondrial mass or an ATP biosynthesis factor. In certain embodiments altered expression of the candidate gene is increased expression, and in certain other embodiments altered expression of the candidate gene is decreased expression.

In another aspect the present invention provides a method of treating a human patient having type 2 diabetes mellitus, comprising administering to the patient an agent that (a) substantially restores to a normal level at least one indicator of glucose responsiveness in cells having reduced glucose responsiveness and reduced mitochondrial mass and/or impaired mitochondrial function; (b) substantially restores to a normal level at least one indicator of mitochondrial function in cells having impaired mitochondrial function; or (c) increases at least one indicator of mitochondrial function to a level above and beyond normal levels in cells having normal mitochondrial function. In addition to being detectable or measurable in intact or permeabilized cells, mitochondrial function may be detected or measured in cellular extracts, isolated mitochondria, submitochondrial particles, or purified mitochondrial components or molecules derived from such cells.

In other embodiments, the present invention provides compositions (e.g., reagents) and methods (e.g., assays such as screening assays or high throughput screens) for identifying therapeutic compositions and/or agents for increasing mitochondrial mass and/or improving mitochondrial function in cells of an animal or human subject. In certain embodiments, candidate compositions and/or agents are screened for their ability to increase mitochondrial mass and/or improve mitochondrial function.

Thus, in certain embodiments the invention provides a method for treating diabetes comprising increasing mitochondrial mass in cells in an individual in need thereof. In certain embodiments such a method for treating diabetes comprises improving mitochondrial function in cells of an individual in need thereof. In certain embodiments the diabetes is type 1 diabetes or type 2 diabetes, and in certain embodiments the cells are pancreatic cells, which in certain further embodiments are pancreatic beta cells. In certain preferred embodiments, mitochondrial mass or mitochondrial activity is increased in response to a NRF or PGC gene or polypeptide.

In another embodiment the present invention provides a method of screening for or identifying an agent that influences the expression of a nucleic acid that encodes a NRF or PGC protein, comprising contacting at least one cell comprising an expression construct with one or more candidate agent and measuring the expression of an NRF protein or a PGC protein. Preferably, the method includes identifying an agent that influences the expression of a nucleic acid that encodes a NRF protein or PGC protein. In certain embodiments, a regulatory element for the expression of the NRF gene or PGC gene is operably linked to a reporter gene, such that the expression of the reporter gene is proportional to or related to the expression of the NRF gene or protein or PGC gene or protein.

In certain embodiments the invention provides a method of screening for or identifying an agent that influences the activity of a NRF protein or PGC protein comprising contacting at least one cell comprising a NRF protein or PGC protein with one or more candidate agent, and measuring the activity of the NRF protein or the PGC protein or measuring one or more mitochondrial activities. In certain other embodiments the invention provides a method of screening for or identifying an agent that influences the activity of a NRF protein or PGC protein, including contacting at least one cell comprising a NRF protein or PGC protein with one or more candidate agents, and measuring at lest one mitochondrial activity.

These and other aspects of the present invention will become evident upon reference to the following detailed description. In addition, various references are set forth herein which describe in more detail certain aspects of this invention, and are therefore incorporated by reference in their entireties.

### BRIEF DESCRIPTION OF THE DRAWINGS

FIG. 1 shows the loss over time of mitochondrial DNA (mtDNA) from INS-1 cells treated with ddC (panel IA) and the secretion of insulin by these cells and the parent INS-1 cells in response to glucose treatment (panel lB).

FIG. 2 shows the results of experiments in which INS-1 cells and mtDNA-depleted INS-1 cells are treated with glucose and measured for their ability to produce ATP (panel 2A) or lactate (panel 2B).

FIG. 3 is a Western blot showing the production of FLAG-huNRF1 protein from a tetracycline-inducible expression construct in the absence (+) or the presence (−) of tetracycline at various timepoints.

FIG. 4 is a gel electrophoretogram showing immunoprecipitated overexpressed recombinant NRF-1.

FIG. 5 shows increased mRNA levels encoding NRF-1,δ-aminolevulinate synthase (ALA-S), NADH dehydrogenase (ND-1) and ATP synthase subunit 6 (ATP6) in tetracycline-induced, Tet^{R} NRF-1 overexpressing SH-SY5Y transfectant cells relative to control, uninduced Tet^{R} SH-SY5Y transfectant cells.

FIG. 6 shows relative RNA hybridization signal intensities from tetracycline-induced, Tet^{R} NRF-1 overexpressing SH-SY5Y transfectant cells relative to control, uninduced Tet^{R} SH-SY5Y transfectant cells using an immobilized human gene array. “Up” in the Ratio column indicates the degree of increase for the indicated gene transcript in induced cells relative to uninduced cells; “down” indicates the degree of decrease for the indicated gene transcript in induced cells relative to uninduced cells.

FIG. 7 shows quantitative analysis of western immunoblot signals obtained following comparative detection of UCP-2 (right) and UCP-3 (left) protein expression in tetracycline-induced, Tet^{R} NRF- 1 overexpressing SH-SY5Y transfectant cells relative to control, uninduced Tet^{R} SH-SY5Y transfectant cells (“con”, control).

### DETAILED DESCRIPTION OF THE INVENTION

The present invention is directed in part to compositions and methods for the alteration of mitochondrial mass and function in the treatment of diseases associated with altered mitochondrial function, and for drug target and drug discovery. In certain embodiments the present invention thus provides several general and useful aspects, including:

(1) a method for treating diabetes that includes increasing mitochondrial mass in cells in an individual in need thereof and a method for treating diabetes that includes altering (and thereby improving) mitochondrial function in cells in an individual in need thereof;

(2) a method of screening for or identifying an agent that influences the expression of a nucleic acid that encodes a NRF or PGC protein that comprises contacting at least one cell comprising an expression construct with one or more candidate agents or test compounds and measuring the expression of an NRF protein or a PGC protein;

(3) a method of screening for or identifying an agent that influences the activity of a NRF protein or PGC protein that comprises contacting at least one cell comprising a NRF protein or PGC protein with one or more candidate agents or test compounds, and measuring the activity of the NRF protein or the PGC protein or measuring one or more mitochondrial activities; and

(4) a method of screening for or identifying an agent that influences the activity of a NRF protein or PGC protein, comprising contacting at least one cell comprising a NRF protein or PGC protein with one or more candidaet agents or test compounds, and measuring at least one mitochondrial activity.

DEFINITIONS AND GENERAL METHODS

Unless defined otherwise, all technical and scientific terms used herein have the same meaning as commonly understood by one of ordinary skill in the art to which this invention is directed. Generally, the nomenclature used herein and the laboratory procedures in cell biology, chemistry, microbiology, molecular biology, cell science, cell culture and tissue culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989)). Where a term is provided in the singular, the inventors also contemplate the plural of that term. The nomenclature used herein and the laboratory procedures described below are those well known and commonly employed in the art.

DEFINITIONS

“Membrane permeant derivative” refers to a chemical derivative of a compound that increases membrane permeability of the compound. These derivatives are made better able to cross cell membranes because hydrophilic groups are masked to provide more hydrophobic derivatives. Also, the making groups can be designed to be cleaved from the compound within a cell to make the compound more hydrophilic once within the cell. Because the substrate is more hydrophilic than the membrane permeant derivative, it preferentially localizes within the cell (U.S. Pat. No. 5,741,657 to Tsien et al., issued Apr. 21, 1998).

“Isolated polynucleotide” refers to a polynucleotide of genomic, cDNA, PCR or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, recombinant RNA, or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source, for example, free of cellular proteins), or (4) is expressed by a cell from a different species, or (5) does not occur in nature.

“Polypeptide” is used herein as a generic term to refer to native protein, fragments, or analogs of a polypeptide sequence.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including viruses, that can be isolated from a source in nature and which has not been intentionally modified by man in the laboratory is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal biding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

“Polynucleotide” refers to a polymeric form of nucleotides of a least ten bases in length, either ribonucleotides or deoxyribonucleotides or a modified from of either type of nucleotide. The term includes single and double stranded forms of DNA or RNA.

“Genomic polynucleotide” refers to a portion of the nuclear genome.

“Mitochondrial genomic polynucleotide” refers to a portion of the mitochondria genome.

“Active genomic polynucleotide” or active portion of a genome refer to regions of a genome (nuclear or mitrochondrial) that can be up regulated, down regulated or both, either directly or indirectly, by a biological process.

“Ribozyme” means enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Specific ribozyme cleavage sites within any potential RNA target are initially identified by scanning the target RNA target for ribozyme cleavage sites which include the sequences GUA, GUU and GUC. Once identified, short RNA sequences between 15 and 20 ribonucleotides corresponding to the region of the target gene containing the cleavage site can be evaluated for secondary structural features which may render the oligonucleotide inoperable. The suitability of candidate targets can also be evaluated by testing accessibility to hybridization with complementary oligonucleotides using ribonuclease protection assays.

“Directly” in the context of a biological process or processes, refers to direct causation of a process that does not require intermediate steps, usually caused by one molecule contacting or binding to another molecule (the same type or different type of molecule). For example, molecule A contacts molecule B, which causes molecule B to exert effect X that is part of a biological process.

“Indirectly” in the context of a biological process or precesses, refers to indirect causation that requires intermediate steps, usually caused by two or more direct steps. For example, molecule A contacts molecule B to exert effect X which in turn causes effect Y.

“Sequence identity” refers to the proportion of base matches between two nucleic acid sequences or the proportion of amino acid matches between two amino acid sequences. When sequence identity is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonuleotides as probes, the sequence identity between the target nucleic acid and the oligonucleotide sequence is preferably not less than 10 target base matches out of 20 (50% identity) and more preferably not less than about 60% identity, 70% identity, 80% identity or 90% identity), and most preferably not less than 95% identity.

“Selectively hybridize” refers to detectably and specifically bind. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence identity between the polynucleotides, oligonucleotides, and fragments thereof and a nucleic acid sequence of interest will be at least 30%, and more typically and preferably of at least 40%, 50%, 60%, 70%, 80% or 90%.

Hybridization and washing conditions are typically performed at high stringency according to conventional hybridization procedures. Positive clones are isolated and sequenced. For example, a full length polynucleotide sequence can be labeled and used as a hybridization probe to isolate genomic clones from an appropriate target library as they are known in the art. Typical hybridization conditions and methods for screening plaque lifts and other purposes are known in the art (Benton and Davis, Science 196:180 (1978); Sambrook et al., supra, (1989)).

Two amino acid sequences have share identity if there is a partial or complete identity between their sequences. For example, 85% identity means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) share identity, as this term is used herein, if they have an alignment score of at least 5 (in standard deviation units) using the program ALIGN with the mutation data matrix and a gap penalty of 6 or greater (Dayhoff, in Atlas of Protein Sequence and Structure, National Biomedical Research Foundation, volume 5, pp. 101-110 (1972) and Supplement 2, pp. 1-10).

“Corresponds to” refers to a polynucleotide sequence that shares identity (for example is identical) to all or a portion of a reference polynucleotide sequence, or that a polypeptide sequence is identical to all or a portion of a reference polypeptide sequence. In contradistinction, the term “complementary to” is used herein to mean that the complementary sequence is homologous to all or a portion of a reference polynucleotide sequence. For illustration, the nucleotide sequence TATAC corresponds to a reference sequence TATAC and is complementary to a reference sequence GTATA.

The following terms are used to describe the sequence relationships between two or more polynucleotides: “reference sequence,” “comparison window,” “sequence identity,” “percentage of sequence identity,” and “substantial identity.” A reference sequence is a defined sequence used as a basis for a sequence comparison; a reference sequence can be a subset of a larger sequence, for example, as a segment of a fall length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (for example a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A comparison window, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window can comprise additions and deletions (for example, gaps) of 20 percent or less as compared to the reference sequence (which would not comprise additions or deletions) for optimal alignment of the two sequences. Optimal alignment of sequences for aligning a comparison window can be conducted by the local identity algorithm (Smith and Waterman, Adv. Appl. Math., 2:482 (1981)), by the identity alignment algorithm (Needleman and Wunsch, J. Mol. Bio., 48:443 (1970)), by the search for similarity method (Pearson and Lipman, Proc. Natl. Acid. Sci. U.S.A. 85:2444 (1988)), by the computerized implementations of these algorithms such as GAP, BESTFIT, FASTA and TFASTA (Wisconsin Genetics Software Page Release 7.0, Genetics Computer Group, Madison, Wis.), or by inspection. Preferably, the best alignment (for example, the result having the highest percentage of identity over the comparison window) generated by the various methods is selected.

“Complete sequence identity” means that two polynucleotide sequences are identical (for example, on a nucleotide-by-nucleotide basis) over the window of comparison.

“Percentage of sequence identity” is calculated by comparing two optimally aligned sequences over the window of comparison, determining the number of positions at which the identical nucleic acid base occurs in both sequences to yield the number of matched positions, dividing the number of matched positions by the total number of positions in the window of comparison (for example, the window size), and multiplying the result by 100 to yield the percentage of sequence identity.

“Substantial identity” as used herein denotes a characteristic of a polynucleotide sequence, wherein the polynucleotide comprises a sequence that has at least 30 percent sequence identity, preferably at least 50 to 60 percent sequence, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or addition which total 20 percent or less of the reference sequence over the window of comparison.

“Substantial identity” as applied to polypeptides herein means that two peptide sequences, when optimally aligned, such as by the programs GAP or BESTFIT using default gap weights, share at least 30 percent sequence identity, preferably at least 40 percent sequence identity, and more preferably at least 50 percent sequence identity, and most preferably at lest 60 percent sequence identity. Preferably, residue positions, which are not identical, differ by conservative amino acid substitutions.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chan is cystein and methionine. Preferred conservative amino acid substitution groups are: valine-leucine-isoleucine; phenylalanine-tyrosine; lysine-arginine; alanine-valine; glutamic-aspartic; and asparagine-glutamine.

“Modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

“Modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonist, partial antagonist, partial agonist, antagonist, antineoplastic, cytotoxic, inhibitors of neoplastic transformation or cell proliferation, cell proliferation promoting agents, antiviral agents, antimicrobial agents, antibacterial agents, antibiotics, and the like) by inclusion in assays described herein. The activity of a modulator may be known, unknown or partially known.

“Test chemical” refers to a chemical or extract to be tested by at least one method of the present invention to be a putative modulator. A test chemical is usually not known to bind to the target of interest. “Control test chemical” refers to a chemical known to bind to the target (for example, a known agonist, antagonist, partial agonist or inverse agonist). Test chemical does not typically include a chemical added to a mixture as a control condition that alters the function of the target to determine signal specificity in an assay. Such control chemicals or conditions include chemicals that (1) non-specifically or substantially disrupt protein structure (for example denaturing agents such as urea or guandium, sulfhydryl reagents such as dithiotritol and beta-mercaptoethanol), (2) generally inhibit cell metabolism (for example mitochondrial uncouples) and (3) non-specifically disrupt electrostatic or hydrophobic interactions of a protein (for example, high salt concentrations or detergents at concentrations sufficient to non-specifically disrupt hydrophobic or electrostatic interactions). The term test chemical also does not typically include chemicals known to be unsuitable for a therapeutic use for a particular indication due to toxicity of the subject. Usually, various predetermined concentrations of test chemicals are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test chemicals, the concentration of test chemical used can be expressed on a weight to volume basis. Under these circumstances, the following ranges of concentrations can be used: between about 0.001 micrograms/ml and about 100 milligram/ml, preferably between about 0.01 micrograms/ml and about 10 milligrams/ml, and more preferably between about 0.1 micrograms/ml and about 1 milligrams/ml or between about 1 microgram/ml and about 100 micrograms/ml.

“Target” refers to a biochemical entity involved in a biological process. Targets are typically proteins that play a useful role in the physiology or biology of an organism. A therapeutic chemical typically binds to a target to alter or modulate its function. As used herein, targets can include, but not be limited to, cell surface receptors, G-proteins, G-protein coupled receptors, kinases, phosphatases, ion channels, lipases, phosholipases, nuclear receptors, intracellular structures, tubules, tubulin, and the like.

“Label” or “labeled” refers to incorporation of a detectable marker, for example by incorporation of a radiolabled compound or attachment to a polypeptide of moieties such as biotin that can be detected by the binding of a section moiety, such as marked avidin. Various methods of labeling polypeptide, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, beta-lactamase, horseradish peroxidase, alkaline phosphatase, luciferase; radioisotopes such as ³H, ¹⁴c, ³⁵s, ¹²⁵I or ¹³¹I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are beta-lactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued Sep. 8, 1998).

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally , as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

“Pharmaceutical agent or drug” refers to a chemical, composition or activity capable of inducing a desired therapeutic effect when property administered by an appropriate dose, regime, route of administration, time and delivery modality.

“Pharmaceutical agent or drug” refers to a chemical, composition or activity capable of inducing a desired therapeutic effect when property administered by an appropriate dose, regime, route of administration, time and delivery modality.

A “bioactive compound” refers to a compound that exhibits at least one bioactivity.

A “bioactivity” refers to a composition that exhibits at least one activity that modulates a biological process, cellular process or disease state. Preferred bioactivities include, but are not limited to activities that modulate at least one mitochondrial activity (such as the production of ATP) or mitochondrial mass, such as by an increase (mitochondrial biogenesis) or decrease in the number of mitrochondria or the amount of mitochondrial DNA. Another preferred bioactivity includes an activity that modulates a cellular process, such as the production or secretion of insulin. A further preferred bioactivity includes an activity that modulates a disease states such as diabetes type I or diabetes type II.

A “mitochondrial biogenesis activity” is an activity that modulates the production of active, inactive or defective mitochondria, preferably active mitochondria.

A “mitoclastic activity” is an activity that modulates the destruction of mitochondria.

An “anti-diabetic activity” is an activity that modulates the disease state of diabetes, including diabetes type I and diabetes type II. Preferably, an anti-diabetic activity is also, directly or indirectly, a mitochondrial biogenesis activity.

A “bioactive derivative” refers to a modification of a bioactive compound or bioactivity that retains at least one characteristic activity of the parent compound.

A “bioactive precursor” refers to a precursor of a bioactive compound or bioactivity that exhibits at least one characteristic activity of the resulting bioactive compound or bioactivity.

A “patient” or “subject” refers a whole organism in need of treatment, such as a farm animal, companion animal or human. An animal refers to any non-human animal.

An “indicator of mitochondrial finction” is any parameter that is indicative of mitochondrial function that can be measured by one skilled in the art. In certain embodiments, the indicator of mitochondrial function is a mitochondrial electron transport chain enzyme, a Krebs cycle enzyme, a mitochondrial matrix component, a mitochondrial membrane component or an ATP biosynthesis factor. In other embodiments, the indicator of mitochondrial function is mitochondrial number per cell or mitochondrial mass per cell. In other embodiments, the indicator of mitochondrial function is an ATP biosynthesis factor. In other embodiments, the indicator of mitochondrial function is the amount of ATP per mitochondrion, the amount of ATP per unit mitochondrial mass, the amount of ATP per unit protein or the amount of ATP per unit mitochondrial protein. In other embodiments, the indicator of mitochondrial function comprises free radical production. In other embodiments, the indicator of mitochondrial function comprises a cellular response to elevated intracellular calcium. In other embodiments, the indicator of mitochondrial function is the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the realtive or absolute amount of mitochondrial DNA per cell in the patient.

“Improving mitochondrial function” or “altering mitochondrial function” may refer to (a) substantially (e.g., in a statistically significant manner, and preferably in a manner that promotes a statistically significant improvement of a clinical parameter such as prognosis, clinical score or outcome) restoring to a normal level at least one indicator of glucose responsiveness in cells having reduced glucose responsiveness and reduced mitochondrial mass and/or impaired mitochondrial function; or (b) substantially (e.g., in a statistically significant manner, and preferably in a manner that promotes a statistically significant improvement of a clinical parameter such as prognosis, clinical score or outcome) restoring to a normal level, or increasing to a level above and beyond normal levels, at least one indicator of mitochondrial function in cells having impaired mitochondrial function or in cells having normal mitochondrial function, respectively. Improved or altered mitochondrial function may result from changes in extramitochondrial structures or events, as well as from mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

“Impaired mitochondrial function” may include a full or partial decrease, inhibition, diminution, loss or other impairment in the level and/or rate of any respiratory, metabolic or other biochemical or biophysical activity in some or all cells of a biological source. As non-limiting examples, markedly impaired ETC activity may be related to impaired mitochondrial function, as may be generation of increased ROS or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of mitochondrial function. These and other non-limiting examples of impaired mitochondrial function are described in greater detail below.

Other technical terms used herein have their ordinary meaning in the art that they are used, as exemplified by a variety of technical dictionaries, such as the McGraw-Hill Dictionary of Chemical Terms and the Stedman's Medical Dictionary.

ASSAYS OF MITOCHONDRIAL NUMBER AND FUNCTION

According to certain embodiments within any of the above aspects of the invention, the indicator of mitochondrial function is a mitochondrial electron transport chain enzyme. In certain embodiments the step of comparing comprises measuring electron transport chain enzyme catalytic activity. In certain embodiments the step of measuring comprises determining enzyme activity per mitochondrion in the sample. In certain embodiments the step of measuring comprises determining enzyme activity per unit of protein in the sample. In certain embodiments the step of comparing comprises measuring electron transport chain enzyme quantity. In certain embodiments the step of measuring comprises determining enzyme quantity per mitochondrion in the sample. In certain embodiments the step of measuring comprises determining enzyme quantity per unit of protein in the sample. In certain embodiments the mitochondrial electron transport chain enzyme comprises at least one subunit of mitochondrial complex 1. In certain embodiments the mitochondrial electron transport chain enzyme comprises at least one subunit of mitochondrial complex II. In certain embodiments the mitochondrial electron transport chain enzyme comprises at least one subunit of mitochondrial complex III. In certain embodiments the mitochondrial electron transport chain enzyme comprises at least one subunit of mitochondrial complex IV. In certain embodiments the at least one subunit of mitochondrial complex IV is COX1, COX2 or COX4. In certain embodiments the mitochondrial electron transport chain enzyme comprises at least one subunit of mitochondrial complex V. In certain embodiments the at least one subunit of mitochondrial complex V is ATP synthase subunit 8 or ATP synthase sub-unit 6.

According to certain other embodiments of the above aspects of the invention, the indicator of mitochondrial function is a mitochondrial matrix component. In certain embodiments the indicator of mitochondrial function is a mitochondrial membrane component. In certain embodiments the mitochondrial membrane component is a mitochondrial inner membrane component. In certain embodiments the mitochondrial membrane component is adenine nucleotide translocator (ANT), voltage dependent anion channel (VDAC), malate-aspartate shuttle, calcium uniporter, UCP-1, UCP-2, UCP-3 (e.g., Boss et al., 2000 Diabetes 49:143; Klingenberg 1999 J. Bioenergetics Biomembranes 31:419), a hexokinase, a peripheral benzodiazepine receptor, a mitochondrial intermembrane creatine kinase, cyclophilin D, a Bc1-2 gene family encoded polypeptide, tricarboxylate carrier or dicarboxylate carrier.

In certain embodiments the indicator of mitochondrial function is a Krebs cycle enzyme. In certain embodiments the step of comparing comprises measuring Krebs cycle enzyme catalytic activity. In certain embodiments the step of measuring comprises determining enzyme activity per mitochondrion in the sample. In certain embodiments the step of measuring comprises determining enzyme activity per unit of protein in the sample. In certain embodiments the step of comparing comprises measuring Krebs cycle enzyme quantity. In certain embodiments the step of measuring comprises determining enzyme quantity per mitochondrion in the sample. In certain embodiments the step of measuring comprises determining enzyme quantity per unit of protein in the sample. In certain embodiments the Krebs cycle enzyme is citrate synthase. In certain embodiments the Krebs cycle enzyme is aconitase, isocitrate dehydrogenase, alpha-ketoglutarate dehydrogenase, succinyl-coenzyme A synthetase, succinate dehydrogenase, fumarase or malate dehydrogenase.

In certain other embodiments of the above aspects of the invention, the indicator of mitochondrial function is mitochondrial mass per cell in the sample. In certain embodiments mitochondrial mass is determined using a mitochondria selective agent. In certain embodiments mitochondrial mass is determined using nonylacridine orange. In certain embodiments mitochondrial mass is determined by morphometric analysis. In certain embodiments the indicator of mitochondrial function is the number of mitochondria per cell in the sample. In certain embodiments the step of comparing comprises measuring a mitochondrion selective reagent. In certain embodiments the mitochondrion selective reagent is fluorescent.

According to certain other embodiments of the above aspects of the invention, the indicator of mitochondrial function is the amount of mitochondrial DNA (“mtDNA”) per cell in the sample. The amount of mitochondrial DNA per cell may be measured and/or expressed in absolute (e.g., mass of mtDNA per cell) or relative (e.g., proportion of mtDNA relative to nuclear DNA) terms. In certain embodiments, mitochondrial DNA is measured by contacting a biological sample containing mitochondrial DNA with an oligonucleotide primer having a nucleotide sequence that is complementary to a sequence present in the mitochondrial DNA, under conditions and for a time sufficient to allow hybridization of the primer to the mitochondrial DNA; and detecting hybridization of the primer to the mitochondrial DNA, and therefrom quantifying the mitochondrial DNA. In certain embodiments the step of detecting comprises a technique that may be polymerase chain reaction, oligonucleotide primer extension assay, ligase chain reaction, or restriction fragment length polymorphism analysis. In certain embodiments, mitochondrial DNA is measured by contacting a sample containing amplified mitochondrial DNA with an oligonucleotide primer having a nucleotide sequence that is complementary to a sequence present in the amplified mitochondrial DNA, under conditions and for a time sufficient to allow hybridization of the primer to the mitochondrial DNA; and detecting hybridization of the primer to the mitochondrial DNA, and therefrom quantifying the mitochondrial DNA. In certain embodiments the step of detecting comprises a technique that may be polymerase chain reaction, oligonucleotide primer extension assay, ligase chain reaction, or restriction fragment length polymorphism analysis. In certain embodiments the mitochondrial DNA is amplified using a technique that may be polymerase chain reaction, transcriptional amplification systems or self-sustained sequence replication. In certain embodiments, mitochondrial DNA is measured by contacting a biological sample containing mitochondrial DNA with an oligonucleotide primer having a nucleotide sequence that is complementary to a sequence present in the mitochondrial DNA, under conditions and for a time sufficient to allow hybridization of the primer to the mitochondrial DNA; and detecting hybridization and extension of the primer to the mitochondrial DNA to produce a product, and therefrom quantifying the mitochondrial DNA. In certain embodiments the step of comparing comprises measuring mitochondrial DNA by contacting a sample containing amplified mitochondrial DNA with an oligonucleotide primer having a nucleotide sequence that is complementary to a sequence present in the amplified mitochondrial DNA, under conditions and for a time sufficient to allow hybridization of the primer to the mitochondrial DNA; and detecting hybridization and extension of the primer to the mitochondrial DNA to produce a product, and therefrom quantifying the mitochondrial DNA. In certain embodiments the mitochondrial DNA is amplified using a technique that may be the polymerase chain reaction (PCR), including quantitaive and competitive PCR (Ahmed et al., BioTechniques 26:290-300, 1999), transcriptional amplification systems or self-sustained sequence replication. In certain embodiments, the amount of mitochondrial DNA in the sample is determined using an oligonucleotide primer extension assay. In other embodiments, the amount of mitochondrial DNA is determined by subjecting a sample to a cesium chloride gradient to separate it from nuclear DNA (see, e.g., Welter et al., Mol. Biol. Rep. 13:17-120, 1988) in the presence of a detectably labeled compound that binds to double-stranded nucleic acids (e.g., ethidium bromide) and comparing the relative and/or absolute signals corresponding to the mitochondrial and nuclear DNAs.

In certain embodiments of any of the above aspects of the invention, the indicator of mitochondrial function is the amount of ATP per cell in the sample. In certain embodiments the step of comparing comprises measuring the amount of ATP per mitochondrion in the sample. In certain embodiments the step of comparing comprises measuring the amount of ATP per unit protein in the sample. In certain embodiments the step of comparing comprises measuring the amount of ATP per unit mitochondrial mass in the sample. In certain embodiments the step of comparing comprises measuring the amount of ATP per unit mitochondial protein in the sample. In certain embodiments the indicator of mitochondrial function is the rate of ATP synthesis in the sample. In certain embodiments the indicator of mitochondrial function is an ATP biosynthesis factor. In certain embodiments the step of comparing comprises measuring ATP biosynthesis factor catalytic activity. In certain embodiments the step of measuring comprises determining ATP biosynthesis factor activity per mitochondrion in the sample. In certain embodiments the step of measuring comprises determining ATP biosynthesis factor activity per unit mitochondrial mass in the sample. In certain embodiments the step of measuring comprises determining ATP biosynthesis factor activity per unit of protein in the sample. In certain embodiments the step of comparing comprises measuring ATP biosynthesis factor quantity. In certain embodiments the step of measuring comprises determining ATP biosynthesis factor quantity per mitochondrion in the sample. In certain embodiments the step of measuring comprises determining ATP biosynthesis factor quantity per unit of protein in the sample.

In certain embodiments of any of the above aspects of the present invention, the indicator of mitochondrial function is free radical production. In certain embodiments the indicator of mitochondrial function is reactive oxygen species, protein nitrosylation, protein carbonyl modification, DNA oxidation, mtDNA oxidation, protein oxidation, protein carbonyl modification, malondialdehyde adducts of proteins, a glycoxidation product, a lipoxidation product, 8′-OH-guanosine adducts or TBARS. In certain embodiments the indicator of mitochondrial function is reactive oxygen species. In certain embodiments the indicator of mitochondrial function is protein nitrosylation. In certain embodiments the indicator of mitochondrial function is DNA oxidation. In certain embodiments the indicator of mitochondrial function is mitochondrial DNA oxidation. In certain embodiments the indicator of mitochondrial function is protein carbonyl modification. In certain embodiments the indicator of mitochondrial function is oxygen consumption, which may be determined according to any of a variety of known methodologies (e.g., Wu et al., 1999 Cell 98:115; Li et al. 1999 J. Biol. Chem. 274:17534).

In yet other certain embodiments of any of the above aspects of the instant invention, the indicator of mitochondrial function is a cellular response to elevated intracellular calcium. In certain other embodiments, the indicator of mitochondrial function is a cellular response to at least one apoptogen.

Without wishing to be bound by theory, impaired mitochondrial function characteristic of type 2 DM may also be related to loss of mitochondrial membrane electrochemical potential by mechanisms other than free radical oxidation, for example by defects in transmitochondrial membrane shuttles and transporters such as the adenine nucleotide transporter or the malate-aspartate shuttle, by intracellular calcium flux, by defects in ATP biosynthesis, by impaired association with porin of hexokinases or other enzymes or by other events. Such collapse of mitochondrial inner membrane potential may result from direct or indirect effects of mitochondrial genes, gene products or related downstream mediator molecules and/or extramitochondrial genes, gene products or related downstream mediators, or from other known or unknown causes.

By way of background, functional mitochondria contain gene products encoded by mitochondrial genes situated in mitochondrial DNA (mtDNA) and by extramitochondrial genes (e.g., nuclear genes) not situated in the circular mitochondrial genome. The 16.5 kb mtDNA encodes 22 tRNAs, two ribosomal RNAs (rRNA) and 13 enzymes of the electron transport chain (ETC), the elaborate multi-complex mitochondrial assembly where, for example, respiratory oxidative phosphorylation takes place. The overwhelming majority of mitochondrial structural and functional proteins are encoded by extramitochondrial, and in most cases presumably nuclear, genes. Accordingly, mitochondrial and extramitochondrial genes may interact directly, or indirectly via gene products and their downstream intermediates, including metabolites, catabolites, substrates, precursors, cofactors and the like. Alterations in mitochondrial function, for example impaired electron transport activity, defective oxidative phosphorylation or increased free radical production, may therefore arise as the result of defective mtDNA, defective extramitochondrial DNA, defective mitochondrial or extramitochondrial gene products, defective downstream intermediates or a combination of these and other factors.

In the most highly preferred embodiments of the invention, an enzyme is the indicator of mitochondrial function as provided herein. The enzyme may be a mitochondrial enzyme, which may further be an ETC enzyme or a Krebs cycle enzyme. The enzyme may also be an ATP biosynthesis factor, which may include an ETC enzyme and/or a Krebs cycle enzyme, or other enzymes or cellular components related to ATP production as provided herein. A “non-enzyme” refers to an indicator of mitochondrial function that is not an enzyme (i.e., that is not a mitochondrial enzyme or an ATP biosynthesis factor as provided herein). In certain other preferred embodiments, an enzyme is a co-indicator of mitochondrial function. The following enzymes may not be indicators of mitochondrial function according to the present invention, but may be co-indicators of mitochondrial function as provided herein: citrate synthase (EC 4.1.3.7), hexokinase II (EC 2.7.1.1; see, e.g., Kruszynska et al. 1998), cytochrome c oxidase (EC 1.9.3.1), phosphofructokinase (EC 2.7.1.11), glyceraldehyde phosphate dehydrogenase (EC 1.2.1.12), glycogen phosphorylase (EC 2.4.1.1) creatine kinase (EC 2.7.3.2), NADH dehydrogenase (EC 1.6.5.3), glycerol 3-phosphate dehydrogenase (EC 1.1.1.8), triose phosphate dehydrogenase (EC 1.2.1.12) and malate dehydrogenase (EC 1.1.1.37).

In other highly preferred embodiments, the indicator of mitochondrial function is any ATP biosynthesis factor as described below. In other preferred embodiments, the indicator is ATP production. In other preferred embodiments, the indicator of mitochondrial function may be mitochondrial mass or mitochondrial number. According to the present invention, mitochondrial DNA content may not be an indicator of mitochondrial finction but may be a co-predictor of mitochondrial function or a co-indicator of mitochondrial fumction, as provided herein. In other preferred embodiments the indicator of mitochondrial function may be free radical production, a cellular response to elevated intracellular calcium or a cellular response to an apoptogen.

INDICATORS OF MITOCHONDRIAL FUNCTION THAT ARE ENZYMES

Certain aspects of the invention are directed to methods that include the detection and/or absolute or relative measurement of at least one indicator of mitochondrial function in biological test samples, wherein the indicator of mitochondrial function is an enzyme. As provided herein, in certain preferred embodiments, such an enzyme may be a mitochondrial enzyme or an ATP biosynthesis factor that is an enzyme, for example an ETC enzyme or a Krebs cycle enzyme.

Reference herein to “enzyme quantity”, “enzyme catalytic activity” or “enzyme expression level” is meant to include a reference to any of a mitochondrial enzyme quantity, activity or expression level or an ATP biosynthesis factor quantity, activity or expression level; either of which may further include, for example, an ETC enzyme quantity, activity or expression level or a Krebs cycle enzyme quantity, activity or expression level. In the most preferred embodiments of the invention, an enzyme is a natural or recombinant protein or polypeptide that has enzyme catalytic activity as provided herein. Such an enzyme may be, by way of non-limiting examples, an enzyme, a holoenzyme, an enzyme complex, an enzyme subunit, an enzyme fragment, derivative or analog or the like, including a truncated, processed or cleaved enzyme.

A “mitochondrial enzyme” that may be an indicator of mitochondrial function as provided herein refers to a mitochondrial molecular component that has enzyme catalytic activity and/or functions as an enzyme cofactor capable of influencing enzyme catalytic activity. As used herein, mitochondria are comprised of “mitochondrial molecular components”, which may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or any covalently or non-covalently complexed combination of these components, or any other biological molecule that is a stable or transient constituent of a mitochondrion.

A mitochondrial enzyme that may be an indicator of mitochondrial function or a co-indicator of mitochondrial function as provided herein, or an ATP biosynthesis factor that may be an indicator of mitochondrial function as provided herein, may comprise an ETC enzyme, which refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC is typically described as comprising complex I (NADH:ubiquinone reductase), complex II (succinate dehydrogenase), complex III (ubiquinone: cytochrome c oxidoreductase), complex IV (cytochrome c oxidase) and complex V (mitochondrial ATP synthetase), where each complex includes multiple polypeptides and cofactors (for review see, e.g., Walker et al., 1995 Meths. Enzymol. 260:14; Emster et al., 1981 J. Cell Biol. 91:227s-255s, and references cited therein).

A mitochondrial enzyme that may be an indicator of mitochondrial function as provided herein, or an ATP biosynthesis factor that may be an indicator of mitochondrial function as provided herein, may also comprise a Krebs cycle enzyme, which includes mitochondrial molecular components that mediate the series of biochemical/bioenergetic reactions also known as the citric acid cycle or the tricarboxylic acid cycle (see, e.g., Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.). Krebs cycle enzymes include subunits and cofactors of citrate synthase, aconitase, isocitrate dehydrogenase, the a-ketoglutarate dehydrogenase complex, succinyl CoA synthetase, succinate dehydrogenase, fumarase and malate dehydrogenase. Krebs cycle enzymes further include enzymes and cofactors that are functionally linked to the reactions of the Krebs cycle, such as, for example, nicotinamide adenine dinucleotide, coenzyme A, thiamine pyrophosphate, lipoamide, guanosine diphosphate, flavin adenine dinucloetide and nucleoside diphosphokinase.

The methods of the present invention also pertain in part to the correlation of type 2 DM with an indicator of mitochondrial function that may be an ATP biosynthesis factor, an altered amount of ATP or an altered amount of ATP production. For example, decreased mitochondrial ATP biosynthesis may be an indicator of mitochondrial finction from which a risk for type 2 DM may be identified.

An “ATP biosynthesis factor” refers to any naturally occurring cellular component that contributes to the efficiency of ATP production in mitochondria. Such a cellular component may be a protein, polypeptide, peptide, amino acid, or derivative thereof, a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like. An ATP biosynthesis factor includes at least the components of the ETC and of the Krebs cycle (see, e.g., Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.) and any protein, enzyme or other cellular component that participates in ATP synthesis, regardless of whether such ATP biosynthesis factor is the product of a nuclear gene or of an extranuclear gene (e.g., a mitochondrial gene). Participation in ATP synthesis may include, but need not be limited to, catalysis of any reaction related to ATP synthesis, transmembrane import and/or export of ATP or of an enzyme cofactor, transcription of a gene encoding a mitochondrial enzyme and/or translation of such a gene transcript.

Compositions and methods for determining whether a cellular component is an ATP biosynthesis factor are well known in the art, and include methods for determining ATP production (including determination of the rate of ATP production in a sample) and methods for quantifying ATP itself. The contribution of an ATP biosynthesis factor to ATP production can be determined, for example, using an isolated ATP biosynthesis factor that is added to cells or to a cell-free system. The ATP biosynthesis factor may directly or indirectly mediate a step or steps in a biosynthetic pathway that influences ATP production. For example, an ATP biosynthesis factor may be an enzyme that catalyzes a particular chemical reaction leading to ATP production. As another example, an ATP biosynthesis factor may be a cofactor that enhances the efficiency of such an enzyme. As another example, an ATP biosynthesis factor may be an exogenous genetic element introduced into a cell or a cell-free system that directly or indirectly affects an ATP biosynthetic pathway. Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, ³²P; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of mitochondrial function. In one embodiment, for instance, ATP may be quantified by measuring luminescence of luciferase catalyzed oxidation of D-luciferin, an ATP dependent process.

“Enzyme catalytic activity” refers to any function performed by a particular enzyme or category of enzymes that is directed to one or more particular cellular function(s). For example, “ATP biosynthesis factor catalytic activity” refers to any function performed by an ATP biosynthesis factor as provided herein that contributes to the production of ATP. Typically, enzyme catalytic activity is manifested as facilitation of a chemical reaction by a particular enzyme, for instance an enzyme that is an ATP biosynthesis factor, wherein at least one enzyme substrate or reactant is covalently modified to form a product. For example, enzyme catalytic activity may result in a substrate or reactant being modified by formation or cleavage of a covalent chemical bond, but the invention need not be so limited. Various methods of measuring enzyme catalytic activity are known to those having ordinary skill in the art and depend on the particular activity to be determined.

For many enzymes, including mitochondrial enzymes or enzymes that are ATP biosynthesis factors as provided herein, quantitative criteria for enzyme catalytic activity are well established. These criteria include, for example, activity that may be defined by international units (IU), by enzyme turnover number, by catalytic rate constant (K_{cat}), by Michaelis-Menten constant (Kₘ), by specific activity or by any other enzymological method known in the art for measuring a level of at least one enzyme catalytic activity. Specific activity of a mitochondrial enzyme, such as an ATP biosynthesis factor, may be expressed as units of substrate detectably converted to product per unit time and, optionally, further per unit sample mass (e.g., per unit protein or per unit mitochondrial mass).

In certain preferred embodiments of the invention, enzyme catalytic activity may be expressed as units of substrate detectably converted by an enzyme to a product per unit time per unit total protein in a sample. In certain particularly preferred embodiments, enzyme catalytic activity may be expressed as units of substrate detectably converted by an enzyme to product per unit time per unit mitochondrial mass in a sample. In certain highly preferred embodiments, enzyme catalytic activity may be expressed as units of substrate detectably converted by an enzyme to product per unit time per unit mitochondrial protein mass in a sample. Products of enzyme catalytic activity may be detected by suitable methods that will depend on the quantity and physicochemical properties of the particular product. Thus, detection may be, for example by way of illustration and not limitation, by radiometric, colorimetric, spectrophotometric, fluorimetric, immunometric or mass spectrometric procedures, or by other suitable means that will be readily apparent to a person having ordinary skill in the art.

In certain embodiments of the invention, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals—Sixth Ed., Molecular Probes, Eugene, Oreg.; Scopes, R. K., Protein Purification: Principles and Practice, 1987, Springer-Verlag, New York; Hermanson, G. T. et al., Immobilized Affinity Ligand Techniques, 1992, Academic Press, Inc., New York; Luo et al., 1998 J. Biotechnol. 65:225 and references cited therein). Spectroscopic methods may be used to detect dyes (including, for example, colorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well known techniques.

As noted above, enzyme catalytic activity of an ATP biosynthesis factor may further include other functional activities that lead to ATP production, beyond those involving covalent alteration of a substrate or reactant. For example by way of illustration and not limitation, an ATP biosynthesis factor that is an enzyme may refer to a transmembrane transporter molecule that, through its enzyme catalytic activity, facilitates the movement of metabolites between cellular compartments. Such metabolites may be ATP or other cellular components involved in ATP synthesis, such as gene products and their downstream intermediates, including metabolites, catabolites, substrates, precursors, cofactors and the like. As another non-limiting example, an ATP biosynthesis factor that is an enzyme may, through its enzyme catalytic activity, transiently bind to a cellular component involved in ATP synthesis in a manner that promotes ATP synthesis. Such a binding event may, for instance, deliver the cellular component to another enzyme involved in ATP synthesis and/or may alter the conformation of the cellular component in a manner that promotes ATP synthesis. Further to this example, such conformational alteration may be part of a signal transduction pathway, an allosteric activation pathway, a transcriptional activation pathway or the like, where an interaction between cellular components leads to ATP production.

Thus, according to the present invention, an ATP biosynthesis may include, for example, a mitochondrial membrane protein. factor Suitable mitochondrial membrane proteins include such mitochondrial components as the adenine nucleotide transporter (ANT; e.g., Fiore et al., 1998 Biochimie 80:137; Klingenberg 1985 Ann. New York Acad. Sci. 456:279), the voltage dependent anion channel (VDAC, also referred to as porin; e.g., Manella, 1997 J. Bioenergetics Biomembr. 29:525), the malate-aspartate shuttle, the mitochondrial calcium uniporter (e.g., Litsky et al., 1997 Biochem. 36:7071), uncoupling proteins (UCP-1, -2, -3; see e.g., Jezek et al., 1998 Int. J. Biochem. Cell Biol. 30:1163), a hexokinase, a peripheral benzodiazepine receptor, a mitochondrial intermembrane creatine kinase, cyclophilin D, a Bcl-2 gene family encoded polypeptide, the tricarboxylate carrier (e.g., Iocobazzi et al., 1996 Biochim. Biophys. Acta 1284:9; Bisaccia et al., 1990 Biochim. Biophys. Acta 1019:250) and the dicarboxylate carrier (e.g., Fiermonte et al., 1998 J. Biol. Chem. 273:24754; Indiveri et al., 1993 Biochim. Biophys. Acta 1143:310; for a general review of mitochondrial membrane transporters, see, e.g., Zonatti et al., 1994 J. Bioenergetics Biomembr. 26:543 and references cited therein).

“Enzyme quantity” as used herein refers to an amount of an enzyme including mitochondrial enzymes or enzymes that are ATP biosynthesis factors as provided herein, or of another ATP biosynthesis factor, that is present, i.e., the physical presence of an enzyme or ATP biosynthesis factor selected as an indicator of mitochondrial function, irrespective of enzyme catalytic activity. Depending on the physicochemical properties of a particular enzyme or ATP biosynthesis factor, the preferred method for determining the enzyme quantity will vary. In the most highly preferred embodiments of the invention, determination of enzyme quantity will involve quantitative determination of the level of a protein or polypeptide using routine methods in protein chemistry with which those having skill in the art will be readily familiar, for example by way of illustration and not limitation, those described in greater detail below.

Accordingly, determination of enzyme quantity may be by any suitable method known in the art for quantifying a particular cellular component that is an enzyme or an ATP biosynthesis factor as provided herein, and that in preferred embodiments is a protein or polypeptide. Depending on the nature and physicochemical properties of the enzyme or ATP biosynthesis factor, determination of enzyme quantity may be by densitometric, mass spectrometric, spectrophotometric, fluorimetric, immunometric, chromatographic, electrochemical or any other means of quantitatively detecting a particular cellular component. Methods for determining enzyme quantity also include methods described above that are useful for detecting products of enzyme catalytic activity, including those measuring enzyme quantity directly and those measuring a detectable label or reporter moiety. In certain preferred embodiments of the invention, enzyme quantity is determined by immunometric measurement of an isolated enzyme or ATP biosynthesis factor. In certain preferred embodiments of the invention, these and other immunological and immunochemical techniques for quantitative determination of biomolecules such as an enzyme or ATP biosynthesis factor may be employed using a variety of assay formats known to those of ordinary skill in the art, including but not limited to enzyme linked immunosorbent assay (ELISA), radioimmunoassay (RIA), immunofluorimetry, immunoprecipitation, equilibrium dialysis, immunodiffusion and other techniques. (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston.) For example, the assay may be performed in a Western blot format, wherein a preparation comprising proteins from a biological sample is submitted to gel electrophoresis, transferred to a suitable membrane and allowed to react with an antibody specific for an enzyme or an ATP biosynthesis factor that is a protein or polypeptide. The presence of the antibody on the membrane may then be detected using a suitable detection reagent, as is well known in the art and described above.

In certain embodiments of the invention, an indicator (or co-indicator) of mitochondrial function including, for example, an enzyme as provided herein, may be present in isolated form. The term “isolated” means that a material is removed from its original environment (e.g., the natural environment if it is naturally occurring). For example, a naturally occurring polypeptide present in a living animal is not isolated, but the same polypeptide, separated from some or all of the co-existing materials in the natural system, is isolated. Such polypeptides could be part of a composition, and still be isolated in that such composition is not part of its natural environment.

Affinity techniques are particularly useful in the context of isolating an enzyme or an ATP biosynthesis factor protein or polypeptide for use according to the methods of the present invention, and may include any method that exploits a specific binding interaction involving an enzyme or an ATP biosynthesis factor to effect a separation. For example, because an enzyme or an ATP biosynthesis factor protein or polypeptide may contain covalently attached oligosaccharide moieties, an affinity technique such as binding of the enzyme (or ATP biosynthesis factor) to a suitable immobilized lectin under conditions that permit carbohydrate binding by the lectin may be a particularly useful affinity technique.

Other useful affinity techniques include immunological techniques for isolating and/or detecting a specific protein or polypeptide antigen (e.g., an enzyme or ATP biosynthesis factor), which techniques rely on specific binding interaction between antibody combining sites for antigen and antigenic determinants present on the factor. Binding of an antibody or other affinity reagent to an antigen is “specific” where the binding interaction involves a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹ . Affinities of binding partners or antibodies can be readily determined using conventional techniques, for example those described by Scatchard et al., Ann. New York Acad. Sci. 51:660 (1949).

Immunological techniques include, but need not be limited to, immunoaffinity chromatography, immunoprecipitation, solid phase immunoadsorption or other immunoaffinity methods. For these and other useful affinity techniques, see, for example, Scopes, R. K., Protein Purification: Principles and Practice, 1987, Springer-Verlag, New York; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques.

As noted above, an indicator of mitochondrial function can be a protein or polypeptide, for example an enzyme or an ATP biosynthesis factor. The protein or polypeptide may be an unmodified polypeptide or may be a polypeptide that has been posttranslationally modified, for example by glycosylation, phosphorylation, fatty acylation including glycosylphosphatidylinositol anchor modification or the like, phospholipase cleavage such as phosphatidylinositol-specific phospholipase c mediated hydrolysis or the like, protease cleavage, dephosphorylation or any other type of protein posttranslational modification such as a modification involving formation or cleavage of a covalent chemical bond.

INDICATORS OF MITOCHONDRIAL FUNCTION THAT ARE MITOCHONDRIAL MASS, MITOCHONDRIAL VOLUME OR MITOCHONDRIAL NUMBER

Certain aspects of the invention are directed to methods that include the detection and/or measurement of at least one indicator of mitochondrial function in biological test samples, wherein the indicator of mitochondrial function is absolute or relative mitochondrial mass, mitochondrial volume or mitochondrial number.

Methods for quantifying mitochondrial mass, volume and/or mitochondrial number are known in the art, and may include, for example, quantitative staining of a representative biological sample. Typically, quantitative staining of mitochondrial may be performed using organelle-selective probes or dyes, including but not limited to mitochondrion selective reagents such as fluorescent dyes that bind to mitochondrial molecular components (e.g., nonylacridine orange, MitoTrackers™) or potentiometric dyes that accumulate in mitochondria as a function of mitochondrial inner membrane electrochemical potential (see, e.g., Haugland, 1996 Handbook of Fluorescent Probes and Research Chemicals—Sixth Ed., Molecular Probes, Eugene, Oreg.). As another example, mitochondrial mass, volume and/or number may be quantified by morphometric analysis (e.g., Cruz-Orive et al., 1990 Am. J. Physiol. 258:L148; Schwerzmann et al., 1986 J. Cell Biol. 102:97). These or any other means known in the art for quantifying mitochondrial mass, volume and/or mitochondrial number in a sample are within the contemplated scope of the invention. For example, the use of such quantitative determinations for purposes of calculating mitochondrial density is contemplated and is not intended to be limiting. In certain highly preferred embodiments, mitochondrial protein mass in a sample is determined using well known procedures. For example, a person having ordinary skill in the art can readily prepare an isolated mitochondrial fraction from a biological sample using established cell fractionation techniques, and therefrom determine protein content using any of a number of protein quantification methodologies well known in the art.

INDICATORS OF MITOCHONDRIAL FUNCTION THAT INCLUDE MITOCHONDRIAL DNA CONTENT

Certain aspects of the invention are directed to methods that include the detection and/or measurement of at least one indicator of mitochondrial function in biological test samples, wherein the indicator of mitochondrial function is the absolute or relative amount of mitochondrial DNA. Quantification of mitochondrial DNA (mtDNA) content may be accomplished by any of a variety of established techniques that are useful for this purpose, including but not limited to oligonucleotide probe hybridization or polymerase chain reaction (PCR) using oligonucleotide primers specific for mitochondrial DNA sequences (see, e.g., Miller et al., 1996 J. Neurochem. 67:1897; Fahy et al., 1997 Nucl. Ac. Res. 25:3102; U.S. patent application Ser. No. 09/098,079; Lee et al., 1998 Diabetes Res. Clin. Practice 42:161; Lee et al., 1997 Diabetes 46(suppl. 1):175A). A particularly useful method is the primer extension assay disclosed by Fahy et al. (Nucl. Acids Res. 25:3102, 1997) and by Ghosh et al. (Am. J. Hum. Genet. 58:325, 1996). Suitable hybridization conditions may be found in the cited references or may be varied according to the particular nucleic acid target and oligonucleotide probe selected, using methodologies well known to those having ordinary skill in the art (see, e.g., Ausubel et al., Current Protocols in Molecular Biology, Greene Publishing, 1987; Sambrook et al., Molecular Cloning: A Laboratory Manual, Cold Spring Harbor Press, 1989).

Examples of other useful techniques for determining the amount of specific nucleic acid target sequences (e.g., mtDNA) present in a sample based on specific hybridization of a primer to the target sequence include specific amplification of target nucleic acid sequences and quantification of amplification products, including but not limited to polymerase chain reaction (PCR, Gibbs et al., Nucl. Ac. Res. 17:2437, 1989), transcriptional amplification systems (e.g., Kwoh et al., 1989 Proc. Nat. Acad. Sci. 86:1173); strand displacement amplification (e.g., Walker et al., Nucl. Ac. Res. 20:1691, 1992; Walker et al., Proc. Nat. Acad. Sci. 89:392, 1992) and self-sustained sequence replication (3SR, see, e.g., Ghosh et al, in Molecular Methods for Virus Detection, 1995 Academic Press, New York, pp. 287-314; Guatelli et al., Proc. Nat. Acad. Sci. 87:1874, 1990), the cited references for which are incorporated herein by reference in their entireties. Other useful amplification techniques include, for example, ligase chain reaction (e.g., Barany, Proc. Nat. Acad. Sci. 88:189, 1991), Q-beta replicase assay (Cahill et al., Clin. Chem. 37:1482, 1991; Lizardi et al., Biotechnol. 6:1197, 1988; Fox et al., J. Clin. Lab. Analysis 3:378, 1989) and cycled probe technology (e.g., Cloney et al., Clin. Chem. 40:656, 1994), as well as other suitable methods that will be known to those familiar with the art.

Sequence length or molecular mass of primer extension assay products may be determined using any known method for characterizing the size of nucleic acid sequences with which those skilled in the art are familiar. In a preferred embodiment, primer extension products are characterized by gel electrophoresis. In another embodiment, primer extension products are characterized by mass spectrometry (MS), which may further include matrix assisted laser desorption ionization/time of flight (MALDI-TOF) analysis or other MS techniques known to those skilled in the art. See, for example, U.S. Pat. Nos. 5,622,824, 5,605,798 and 5,547,835. In another embodiment, primer extension products are characterized by liquid or gas chromatography, which may further include high performance liquid chromatography (HPLC), gas chromatography-mass spectrometry (GC-MS) or other well known chromatographic methodologies.

INDICATORS OF MITOCHONDRIAL FUNCTION THAT ARE CELLULAR RESPONSES TO ELEVATED INTRACELLULAR CALCIUM

Certain aspects of the present invention, as it relates detecting and/or measuring an indicator of mitochondrial function, involve monitoring intracellular calcium homeostasis and/or cellular responses to perturbations of this homeostasis, including physiological and pathophysiological calcium regulation. The range of cellular responses to elevated intracellular calcium is broad, as is the range of methods and reagents for the detection of such responses. Many specific cellular responses are known to those having ordinary skill in the art; these responses will depend on the particular cell types present in a selected biological sample. As non-limiting examples, cellular responses to elevated intracellular calcium include secretion of specific secretory products, exocytosis of particular pre-formed components, increased glycogen metabolism and cell proliferation (see, e.g., Clapham, 1995 Cell 80:259; Cooper, The Cell—A Molecular Approach, 1997 ASM Press, Washington, D.C.; Alberts, B., Bray, D., et al., Molecular Biology of the Cell, 1995 Garland Publishing, New York).

As a brief background, normal alterations of intramitochondrial Ca²⁺ are associated with normal metabolic regulation (Dykens, 1998 in Mitochondria & Free Radicals in Neurodegenerative Diseases, Beal, Howell and Bodis-Wollner, Eds., Wiley-Liss, New York, pp. 29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1991, Am. J. Physio. 27: C755; Gunter et al., 1994, Am. J. Physiol. 267:313). For example, fluctuating levels of mitochondrial free Ca²⁺ may be responsible for regulating oxidative metabolism in response to increased ATP utilization, via allosteric regulation of enzymes (reviewed by Crompton et al., 1993 Basic Res. Cardiol. 88: 513-523;) and the glycerophosphate shuttle (Gunter et al., 1994 J. Bioenerg. Biomembr. 26: 471).

Normal mitochondrial function includes regulation of cytosolic free calcium levels by sequestration of excess Ca²⁺ within the mitochondrial matrix. Depending on cell type, cytosolic Ca²⁺ concentration is typically 50-100 nM. In normally functioning cells, when Ca²⁺ levels reach 200-300 nM, mitochondria begin to accumulate Ca²⁺ as a function of the equilibrium between influx via a Ca²⁺ uniporter in the inner mitochondrial membrane and Ca²⁺ efflux via both Na⁺ dependent and Na⁺ independent calcium carriers. In certain instances, such perturbation of intracellular calcium homeostasis is a feature of diseases (such as type 2 DM) associated with mitochondrial function, regardless of whether the calcium regulatory dysfunction is causative of, or a consequence of, mitochondrial function.

Elevated mitochondrial calcium levels thus may accumulate in response to an initial elevation in cytosolic free calcium, as described above. Such elevated mitochondrial calcium concentrations in combination with reduced ATP or other conditions associated with mitochondrial pathology, can lead to collapse of mitochondrial inner membrane potential (see Gunter et al., 1998 Biochim. Biophys. Acta 1366:5; Rottenberg and Marbach, 1990, Biochim. Biophys. Acta 1016:87). Generally, in order to practice the subject invention method for identifying a risk for type 2 DM in an individual, the extramitochondrial (cytosolic) level of Ca²⁺ in a biological sample is greater than that present within mitochondria. In the case of type 2 DM, mitochondrial or cytosolic calcium levels may vary from the above ranges and may range from, e.g., about 1 nM to about 500 mM, more typically from about 10 nM to about 100 mM and usually from about 20 nM to about 1 mM, where “about” indicates ±10%. A variety of calcium indicators are known in the art, including but not limited to, for example, fura-2 (McCormack et al., 1989 Biochim. Biophys. Acta 973:420); mag-fura-2; BTC (U.S. Pat. No. 5,501,980); fluo-3, fluo-4 and fluo-5N (U.S. Pat. No. 5,049,673); rhod-2; benzothiaza-1; and benzothiaza-2 (all of which are available from Molecular Probes, Eugene, Oreg.). These or any other means for monitoring intracellular calcium are contemplated according to the subject invention method for identifying a risk for type 2 DM.

For monitoring an indicator of mitochondrial function that is a cellular response to elevated intracellular calcium, compounds that induce increased cytoplasmic and mitochondrial concentrations of Ca²⁺, including calcium ionophores, are well known to those of ordinary skill in the art, as are methods for measuring intracellular calcium and intramitochondrial calcium (see, e.g., Gunter and Gunter, 1994 J. Bioenerg. Biomembr. 26: 471; Gunter et al., 1998 Biochim. Biophys. Acta 1366:5; McCormack et al., 1989 Biochim. Biophys. Acta 973:420; Orrenius and Nicotera, 1994 J. Neural. Transm. Suppl. 43:1; Leist and Nicotera, 1998 Rev. Physiol. Biochem. Pharmacol. 132:79; and Haugland, 1996 Handbook of Fluorescent Probes and Research Chemicals—Sixth Ed., Molecular Probes, Eugene, Oreg.). Accordingly, a person skilled in the art may readily select a suitable ionophore (or another compound that results in increased cytoplasmic and/or mitochondrial concentrations of Ca²⁺) and an appropriate means for detecting intracellular and/or intramitochondrial calcium for use in the present invention, according to the instant disclosure and to well known methods.

Ca²⁺ influx into mitochondria appears to be largely dependent, and may be completely dependent, upon the negative transmembrane electrochemical potential (DY) established at the inner mitochondrial membrane by electron transfer, and such influx fails to occur in the absence of DY even when an eight-fold Ca²⁺ concentration gradient is imposed (Kapus et al., 1991 FEBS Lett. 282:61). Accordingly, mitochondria may release Ca²⁺ when the membrane potential is dissipated, as occurs with uncouplers like 2,4-dinitrophenol and carbonyl cyanide p-trifluoro-methoxyphenylhydrazone (FCCP). Thus, according to certain embodiments of the present invention, collapse of DY may be potentiated by influxes of cytosolic free calcium into the mitochondria, as may occur under certain physiological conditions including those encountered by cells of a subject having type 2 DM. Detection of such collapse may be accomplished by a variety of means as provided herein.

Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Ernster et al., 1981 J. Cell Biol. 91:227s and references cited; see also Haugland, 1996 Handbook of Fluorescent Probes and Research Chemicals—Sixth Ed., Molecular Probes, Eugene, Oreg., pp. 266-274 and 589-594.). For example, by way of illustration and not limitation, the fluorescent probes 2-,4-dimethylaminostyryl-N-methyl pyridinium (DASPMI) and tetramethylrhodamine esters (e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, at http://www.probes.com/handbook/toc.html). Other fluorescent detectable compounds that may be used in the invention include but are not limited to rhodamine 123, rhodamine B hexyl ester, DiOC₆(3), JC-1 [5,5′,6,6′-Tetrachloro-1,1′,3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, G_ttingen, Germany; see Sakanoue et al., 1997 J. Biochem. 121:29). Methods for monitoring mitochondrial membrane potential are also disclosed in U.S. patent application Ser. No. 09/161,172.

Mitochondrial membrane potential can also be measured by non-fluorescent means, for example by using TTP (tetraphenylphosphonium ion) and a TTP-sensitive electrode (Kamo et al., 1979 J. Membrane Biol. 49:105; Porter and Brand, 1995 Am. J. Physiol. 269:RI213). Those skilled in the art will be able to select appropriate detectable compounds or other appropriate means for measuring DYm. By way of example and not limitation, TMRM is somewhat preferable to TMRE because, following efflux from mitochondria, TMRE yields slightly more residual signal in the endoplasmic reticulicum and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein.)

Exquisite sensitivity to extraordinary mitochondrial accumulations of Ca²⁺ that result from elevation of intracellular calcium, as described above, may also characterize type 2 DM. Such mitochondrial sensitivity may provide an indicator of mitochondrial finction according to the present invention. Additionally, a variety of physiologically pertinent agents, including hydroperoxide and free radicals, may synergize with Ca²⁺ to induce collapse of DY (Novgorodov et al., 1991 Biochem. Biophys. Acta 1058: 242; Takeyama et al., 1993 Biochem. J. 294: 719; Guidox et al., 1993 Arch. Biochem. Biophys. 306:139).

INDICATORS OF MITOCHONDRIAL FUNCTION THAT INCLUDE RESPONSES TO APOPTOGENIC STIMULI

Turning to another aspect, the present invention relates to the detection and/or measurement of an indicator of mitochondrial function, wherein the mitochondrial function involves programmed cell death or apoptosis. The range of responses to various known apoptogenic stimuli is broad, as is the range of methods and reagents for the detection of such responses.

By way of background, mitochondrial dysfumction is thought to be critical in the cascade of events leading to apoptosis in various cell types (Kroemer et al., FASEB J 9:1277-87, 1995). Mitochondrial physiology may be among the earliest events in programmed cell death (Zamzami et al., J. Exp. Med. 182:367-77, 1995; Zamzami et al., J. Exp. Med. 181:1661-72, 1995) and elevated reactive oxygen species (ROS) levels that result from such mitochondrial function may initiate the apoptotic cascade (Ausserer et al., Mol Cell Biol 14:5032-42, 1994). In several cell types, reduction in the mitochondrial membrane potential (DYm) precedes the nuclear DNA degradation that accompanies apoptosis. In cell-free systems, mitochondrial, but not nuclear, enriched fractions are capable of inducing nuclear apoptosis (Newmeyer et al., Cell 70:353-64, 1994). Perturbation of mitochondrial respiratory activity leading to altered cellular metabolic states, such as elevated intracellular ROS, may occur in type 2 DM and may further induce pathogenetic events via apoptotic mechanisms.

Oxidatively stressed mitochondria may release a pre-formed soluble factor that can induce chromosomal condensation, an event preceding apoptosis (Marchetti et al., Cancer Res. 56:2033-38, 1996). In addition, members of the Bcl-2 family of anti-apoptosis gene products are located within the outer mitochondrial membrane (Monaghan et al., J. Histochem. Cytochem. 40:1819-25, 1992) and these proteins appear to protect membranes from oxidative stress (Korsmeyer et al., Biochim. Biophys. Act. 1271:63, 1995). Localization of Bcl-2 to this membrane appears to be indispensable for modulation of apoptosis (Nguyen et al., J. Biol. Chem. 269:16521-24, 1994). Thus, changes in mitochondrial physiology may be important mediators of apoptosis.

Impaired mitochondrial function may therefore be reflected in a lower threshold for induction of apoptosis by one or more apoptogens. A variety of apoptogens are known to those familiar with the art (see, e.g., Green et al., 1998 Science 281:1309 and references cited therein) and may include by way of illustration and not limitation: tumor necrosis factor-alpha (TNF-a); Fas ligand; glutamate; N-methyl-D-aspartate (NMDA); interleukin-3 (IL-3); herbimycin A (ManciniÊtet al., 1997 J. Cell. Biol. 138:449-469); paraquat (Costantini et al., 1995 Toxicology 99:1-2); ethylene glycols; protein kinase inhibitors, e.g., staurosporine, calphostin C, caffeic acid phenethyl ester, chelerythrine chloride, genistein; 1-(5-isoquinolinesulfonyl)-2-methylpiperazine; KN-93; N-[2-((p-bromocinnamyl)amino)ethyl]-5-5-isoquinolinesulfonamide; quercitin; d-erythrosphingosine derivatives; UV irradiation; ionophores, e.g.: ionomycin and valinomycin; MAP kinase inducers, e.g.: anisomycin, anandamine; cell cycle blockers, e.g.: aphidicolin, colcemid, 5-fluorouracil, homoharringtonine; acetylcholinesterase inhibitors, e.g. berberine; anti-estrogens, e.g.: tamoxifen; pro-oxidants, e.g.,: tert-butyl peroxide, hydrogen peroxide; free radicals, e.g., nitric oxide; inorganic metal ions, e.g., cadmium; DNA synthesis inhibitors, e.g.: actinomycin D; DNA intercalators, e.g., doxorubicin, bleomycin sulfate, hydroxyurea, methotrexate, mitomycin C, camptothecin, daunorubicin; protein synthesis inhibitors, e.g., cycloheximide, puromycin, rapamycin; agents that affect microtubulin formation or stability, e.g.: vinblastine, vincristine, colchicine, 4-hydroxyphenylretinamide, paclitaxel; Bad protein, Bid protein and Bax protein (see, e.g., Jurgenmeier et al., 1998 Proc. Nat. Acad. Sci. USA 95:4997-5002 and references cited therein); calcium and inorganic phosphate (Kroemer et al., 1998 Ann. Rev. Physiol 60:619).

In one embodiment of the subject invention method wherein the indicator of mitochondrial function is a cellular response to an apoptogen, cells in a biological sample that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by a person having ordinary skill in the art, for example by using light microscopy. As another example, cells undergoing apoptosis may exhibit fragmentation and disintegration of chromosomes, which may be apparent by microscopy and/or through the use of DNA-specific or chromatin-specific dyes that are known in the art, including fluorescent dyes. Such cells may also exhibit altered plasma membrane permeability properties as may be readily detected through the use of vital dyes (e.g., propidium iodide, trypan blue) or by the detection of lactate dehydrogenase leakage into the extracellular milieu. These and other means for detecting apoptotic cells by morphologic criteria, altered plasma membrane permeability and related changes will be apparent to those familiar with the art.

In another embodiment of the subject invention method wherein the indicator of mitochondrial function is a cellular response to an apoptogen, cells in a biological sample may be assayed for translocation of cell membrane phosphatidylserine (PS) from the inner to the outer leaflet of the plasma membrane, which may be detected, for example, by measuring outer leaflet binding by the PS-specific protein annexin. (Martin et al., J. Exp. Med. 182:1545, 1995; Fadok et al., J. Immunol. 148:2207, 1992.) In still another embodiment of this aspect of the invention, a cellular/biochemical response to an apoptogen is determined by an assay for induction of specific protease activity in any member of a family of apoptosis-activated proteases known as the caspases (see, e.g., Green et al., 1998 Science 281:1309). Those having ordinary skill in the art will be readily familiar with methods for determining caspase activity, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., 1997 J. Neurosci. 17:6165). The synthetic peptide Z-Tyr-Val-Ala-Asp-AFC (SEQ ID NO:13), wherein “Z” indicates a benzoyl carbonyl moiety and “AFC” indicates 7-amino-4-trifluoromethylcoumarin (Kluck et al., 1997 Science 275:1132; Nicholson et al., 1995 Nature 376:37), is one such substrate. Other non-limiting examples of substrates include nuclear proteins such as U1-70 kDa and DNA-PKcs (Rosen and Casciola-Rosen, 1997 J. Cell. Biochem. 64:50; Cohen, 1997 Biochem. J. 326:1).

As described above, the mitochondrial inner membrane may exhibit highly selective and regulated permeability for many small solutes, but is impermeable to large (>˜10 kDa) molecules. (See, e.g., Quinn, 1976 The Molecular Biology of Cell Membranes, University Park Press, Baltimore, Md.). In cells undergoing apoptosis, however, collapse of mitochondrial membrane potential may be accompanied by increased permeability permitting macromolecule diffusion across the mitochondrial membrane. Thus, in another embodiment of the subject invention method wherein the indicator of mitochondrial function is a cellular response to an apoptogen, detection of a mitochondrial protein, for example cytochrome c that has escaped from mitochondria in apoptotic cells, may provide evidence of a response to an apoptogen that can be readily determined. (Liu et al., Cell 86:147, 1996) Such detection of cytochrome c may be performed spectrophotometrically, immunochemically or by other well established methods for determining the presence of a specific protein.

For instance, release of cytochrome c from cells challenged with apoptotic stimuli (e.g., ionomycin, a well known calcium ionophore) can be followed by a variety of immunological methods. Matrix-assisted laser desorption ionization time-of-flight (MALDI-TOF) mass spectrometry coupled with affinity capture is particularly suitable for such analysis since apo-cytochrome c and holo-cytochrome c can be distinguished on the basis of their unique molecular weights. For example, the Surface-Enhanced Laser Desorption/Ionization (SELDI™) system (Ciphergen, Palo Alto, Calif.) may be utilized to detect cytochrome c release from mitochondria in apoptogen treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular mass of the protein is determined by its time of flight to the detector of the SELDI™ mass spectrometer.

A person having ordinary skill in the art will readily appreciate that there may be other suitable techniques for quantifying apoptosis, and such techniques for purposes of determining an indicator of mitochondrial function that is a cellular response to an apoptogenic stimulus are within the scope of the methods provided by the present invention.

FREE RADICAL PRODUCTION As AN INDICATOR OF MITOCHONDRIAL FUNCTION

In certain embodiments of the present invention, free radical production in a biological sample may be detected as an indicator of mitochondrial function. Although mitochondria are a primary source of free radicals in biological systems (see, e.g., Murphy et al., 1998 in Mitochondria and Free Radicals in Neurodegenerative Diseases, Beal, Howell and Bodis-Wollner, Eds., Wiley-Liss, New York, pp. 159-186 and references cited therein), the invention should not be so limited and free radical production can be an indicator of mitochondrial function regardless of the particular subcellular source site. For example, numerous intracellular biochemical pathways that lead to the formation of radicals through production of metabolites such as hydrogen peroxide, nitric oxide or superoxide radical via reactions catalyzed by enzymes such as flavin-linked oxidases, superoxide dismutase or nitric oxide synthetase, are known in the art, as are methods for detecting such radicals (see, e.g., Kelver, 1993 Crit. Rev. Toxicol. 23:21; Halliwell B. and J. M. C. Gutteridge, Free Radicals in Biology and Medicine, 1989 Clarendon Press, Oxford, UK; Davies, K. J. A. and F. Ursini, The Oxygen Paradox, Cleup Univ. Press, Padova, IT). Mitochondrial function, such as failure at any step of the ETC, may also lead to the generation of highly reactive free radicals. As noted above, radicals resulting from mitochondrial function include reactive oxygen species (ROS), for example, superoxide, peroxynitrite and hydroxyl radicals, and potentially other reactive species that may be toxic to cells. Accordingly, in certain preferred embodiments of the invention an indicator of mitochondrial function may be a detectable free radical species present in a biological sample. In certain particularly preferred embodiments, the detectable free radical will be a ROS.

Methods for detecting a free radical that may be useful as an indicator of mitochondrial finction are known in the art and will depend on the particular radical. Typically, a level of free radical production in a biological sample may be determined according to methods with which those skilled in the art will be readily familiar, including but not limited to detection and/or measurement of: glycoxidation products including pentosidine, carboxymethylysine and pyrroline; lipoxidation products including glyoxal, malondialdehyde and 4-hydroxynonenal; thiobarbituric acid reactive substances (TBARS; see, e.g., Steinbrecher et al., 1984 Proc. Nat. Acad. Sci. USA 81:3883; Wolff, 1993 Br. Med. Bull. 49:642) and/or other chemical detection means such as salicylate trapping of hydroxyl radicals (e.g., Ghiselli et al., 1998 Meths. Mol. Biol. 108:89; Halliwell et al., 1997 Free Radic. Res. 27:239) or specific adduct formation (see, e.g., Mecocci et al. 1993 Ann. Neurol. 34:609; Giulivi et al., 1994 Meths. Enzymol. 233:363) including malondialdehyde formation, protein nitrosylation, DNA oxidation including mitochondrial DNA oxidation, 8Õ-OH-guanosine adducts (e.g., Beckman et al., 1999 Mutat. Res. 424:51), protein oxidation, protein carbonyl modification (e.g., Baynes et al., 1991 Diabetes 40:405; Baynes et al., 1999 Diabetes 48:1); electron spin resonance (ESR) probes; cyclic voltametry; fluorescent and/or chemiluminescent indicators (see also e.g., Greenwald, R. A. (ed.), Handbook of Methods for Oxygen Radical Research, 1985 CRC Press, Boca Raton, Fla.; Acworth and Bailey, (eds.), Handbook of Oxidative Metabolism, 1995 ESA, Inc., Chelmsford, Mass.; Yla-Herttuala et al., 1989 J. Clin. Invest. 84:1086; Velazques et al., 1991 Diabetic Medicine 8:752; Belch et al., 1995 Int. Angiol. 14:385; Sato et al., 1979 Biochem. Med. 21:104; Traverso et al., 1998 Diabetologia 41:265; Haugland, 1996 Handbook of Fluorescent Probes and Research Chemicals—Sixth Ed., Molecular Probes, Eugene, Oreg., pp. 483-502, and references cited therein). For example, by way of illustration and not limitation, oxidation of the fluorescent probes dichlorodihydrofluorescein diacetate and its carboxylated derivative carboxydichlorodihydrofluorescein diacetate (see, e.g., Haugland, 1996, supra) may be quantified following accumulation in cells, a process that is dependent on, and proportional to, the presence of reactive oxygen species (see also, e.g., Molecular Probes On-line Handbook of Fluorescent Probes and Research Chemicals, at http://www.probes.com/handbook/toc.html). Other fluorescent detectable compounds that may be used in the invention for detection of free radical production include but are not limited to dihydrorhodamine and dihydrorosamine derivatives, cis-parinaric acid, resorufin derivatives, lucigenin and any other suitable compound that may be known to those familiar with the art.

Thus, as also described above, free radical mediated damage may inactivate one or more of the myriad proteins of the ETC and in doing so, may uncouple the mitochondrial chemiosmotic mechanism responsible for oxidative phosphorylation and ATP production. Indicators of mitochondrial function that are ATP biosynthesis factors, including determination of ATP production, are described in greater detail herein. Free radical mediated damage to mitochondrial functional integrity is also just one example of multiple mechanisms associated with mitochondrial fuction that may result in collapse of the electrochemical potential maintained by the inner mitochondrial membrane. Methods for detecting changes in the inner mitochondrial membrane potential are described above and in co-pending U.S. patent application Ser. No. 09/161,172.

SAMPLES

Samples of cells for the present invention can be provided as cells in culture or from a subject, such as a tissue, fluid or organ or a portion of any of the foregoing. For example, cells can preferably be from tissues that are involved in glucose metabolism, such as pancreatic cells, islates of Langerhans, pancreatic beta cells, muscle cells, liver cells or other appropriate cells. Preferably, cells are provided in culture and can be a primary cell line or a continuous cell line and can be provided as a clonal population of cells or a mixed population of cells. Preferably, the cells are insulin producing (and more preferably insulin secreting) cells in that they naturally produce and optionally secrete insulin or have been engineered to produce and optionally secrete insulin under appropriate stimuli, such as in the presence of Glucose.

Preferred cells include, but are not limited to, a glucose-responsive, insulin-producing cell line such as the rat-derived INS-1 cell line; cells (particularly beta cells) derived from Zucker diabetic fatty rat (ZDF) or cells (particularly beta cells) from Zucker lean control rates (ZLC) ) (Shafrir et al., J. Basic Clin. PhysioL Pharmacol. 9:347-385, 1988). Other preferred cells include derivatives of the above cell lines that have been depleted of their mitochondrial DNA (mtDNA); such cells are commonly referred to as “ρ⁰” (“rho-zero”). Other preferred cells include cybrid cells, i.e., derivatives of the above cell lines in which the endogenous mtDNA has been replaced by mtDNA from an individual suffering from diabetes or another mitochondrial disease of interest. General methods for preparing, using and assaying the mitochondrial functions of rho-zero and cybrid cells are described in U.S. Pat. No. 5,888,438, published PCT applications WO 95/26973 and WO 98/17826, King and Attardi (Science 246:500-503, 1989), Chomyn et al. (Mol. Cell. Biol. 11:2236-2244, 1991), Miller et al. (J. Neurochem. 67:1897-1907, 1996), Swerdlow et al. (Annals of Neurology 40:663-671, 1996), Cassarino et al. (Biochim. Biophys. Acta 1362:77-86, 1997), Swerdlow et al. (Neurology 49:918-925, 1997), Sheehan et al. (J. Neurochem. 68:1221-1233, 1997), and Sheehan et al. (J. Neurosci. 17:4612-4622, 1997). Cybrid cells comprising mitochondria dervied from diabetic individuals are described in published PCT applications WO 95/26973 and WO 98/17826.

Cybrid cells can be made using mitochondria from healthy subjects or from subjects that may have mitochondrial defects. Briefly, a host cell line is treated with ethidium bromide, or an antiviral agent (as described in copending U.S. patent applications Ser. Nos. 09/069,489 and 09/237,999) such as ddC, to substantially deplete cells of mitochondrial DNA (mtDNA). Platelets, or other sources of mitochondria, are fused with the mitochondria depleted cells to form a hybrid cell that includes the nuclear genome of the host cell and the mitochondria (and thus mitochondrial genome) of the subject.

In the beta cells of ZDF rats, increased ceraminde synthesis and nitric oxide increases beta cell apoptosis. Cermaide (particularly C2 ceramide, but not C2 dihydroceramide) and nitric oxide are stimulated by FAA (oleate:palmitate). Also, C6 ceramide can induce casepase 3 activation in INS-1 cells. Furthermore, sodium nitroprusside (SNP) can induce INS-1 cell death.

Biological samples may comprise any tissue or cell preparation in which at least one candidate indicator of mitochondrial function can be detected, and may vary in nature accordingly, depending on the particular indicator(s) to be compared. Biological samples may be provided by obtaining a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiatable cell lines, transformed cell lines and the like. In certain preferred embodiments of the invention, the subject or biological source may be suspected of having or being at risk for having type 2 diabetes mellitus, and in certain preferred embodiments of the invention the subject or biological source may be known to be free of a risk or presence of such as disease.

In certain other preferred embodiments where it is desirable to determine whether or not a subject or biological source falls within clinical parameters indicative of type 2 diabetes mellitus, signs and symptoms of type 2 diabetes that are accepted by those skilled in the art may be used to so designate a subject or biological source, for example clinical signs referred to in Gavin et al. (Diabetes Care 22(suppl. 1):S5-S19, 1999, American Diabetes Association Expert Committee on the Diagnosis and Classification of Diabetes Mellitus) and references cited therein, or other means known in the art for diagnosing type 2 diabetes.

In certain aspects of the invention, biological samples containing at least one candidate indicator (or co-indicator as provided herein) of mitochondrial function may be obtained from the subject or biological source before and after contacting the subject or biological source with a candidate agent, for example to identify a candidate agent capable of effecting a change in the level of the indicator (or co-indicator) of mitochondrial function as defined above, relative to the level before exposure of the subject or biological source to the agent. The indicator (or co-indicator) may optionally, in certain preferred embodiments wherein the indicator (or co-indicator) is an enzyme or an ATP biosynthesis factor, be determined as a measure of enzyme (or ATP biosynthesis factor) catalytic activity in the sample, as a measure of enzyme (or ATP biosynthesis factor) quantity in the sample or as a measure of enzyme (or ATP biosynthesis factor) expression level in the sample, as provided herein.

In a most preferred embodiment of the invention, the biological sample containing at least one candidate indicator (or co-indicator) of mitochondrial function comprises a skeletal muscle biopsy. In another preferred embodiment of the invention, the biological sample containing at least one candidate indicator (or co-indicator) of mitochondrial function may comprise whole blood, and may in another preferred embodiment comprise a crude buffy coat fraction of whole blood, which is known in the art to comprise further a particulate fraction of whole blood enriched in white blood cells and platelets and substantially depleted of erythrocytes. Those familiar with the art will know how to prepare such a buffy coat fraction, which may be prepared by differential density sedimentation of blood components under defined conditions, including the use of density dependent separation media, or by other methods. In other preferred embodiments, the biological sample containing at least one indicator (or co-indicator) of mitochondrial finction may comprise an enriched, isolated or purified blood cell subpopulation fraction such as, for example, lymphocytes, polymorphonuclear leukocytes, granulocytes and the like. Methods for the selective preparation of particular hematopoietic cell subpopulations are well known in the art (see, e.g., Current Protocols in Immunology, J. E. Coligan et al., (Eds.) 1998 John Wiley & Sons, New York).

According to certain embodiments of the invention, the particular cell type or tissue type from which a biological sample is obtained may influence qualitative or quantitative aspects of at least one candidate indicator (or co-indicator) of mitochondrial function contained therein, relative to the corresponding candidate indicator (or co-indicator) of mitochondrial function obtained from distinct cell or tissue types of a common biological source. It is therefore within the contemplation of the invention to quantify at least one candidate indicator (or co-indicator) of mitochondrial function in biological samples from different cell or tissue types as may render the advantages of the invention most useful for type 2 diabetes mellitus, and further for a particular degree of progression of known or suspected type 2 diabetes. The relevant cell or tissue types will be known to those familiar with such diseases.

For example, as provided herein, skeletal muscle may represent a particularly preferred tissue type in which oxidative energy demand (e.g., ATP demand) is high and is requried for normal glucose utilization. Accordingly, other biological samples derived from cell or tissue types that use mitochondrial ATP for cellular functions involved in glucose homeostasis, for example pancreatic beta cells and adipose cells, may also be particularly useful.

In order to determine whether a mitochondrial alteration may contribute to a particular disease state, it may be useful to construct a model system for diagnostic tests and for screening candidate therapeutic agents in which the nuclear genetic background may be held constant while the mitochondrial genome is modified. It is known in the art to deplete mitochondrial DNA from cultured cells to produce r⁰ cells, thereby preventing expression and replication of mitochondrial genes and inactivating mitochondrial function. It is further known in the art to repopulate such r⁰ cells with mitochondria derived from foreign cells in order to assess the contribution of the donor mitochondrial genotype to the respiratory phenotype of the recipient cells. Such cytoplasmic hybrid cells, containing genomic and mitochondrial DNAs of differing biological origins, are known as cybrids. See, for example, International Publication Number WO 95/26973 and U.S. Pat. No. 5,888,498 which are hereby incorporated by reference in their entireties, and references cited therein.

According to the present invention, a level of at least one indicator (or co-indicator) of mitochondrial function is determined in a biological sample from a subject or biological source. For subjects that are asymptomatic, that exhibit IGT or that meet clinical criteria for having or being at risk for having type 2 DM (Gavin et al. Diabetes Care 22(suppl. l):S5-S19, 1999, American Diabetes Association Expert Committee on the Diagnosis and Classification of Diabetes Mellitus), such determination may have prognostic and/or diagnostic usefulness. For example, where other clinical indicators of type 2 DM are known, levels of at least one indicator of mitochondrial function in subjects known to be free of a risk or presence of type 2 DM based on the absence of these indicators may be determined to establish a control range for such level(s). The levels may also be determined in biological samples obtained from subjects suspected of having or being at risk for having type 2 DM, and compared to the control range determined in disease free subjects. Those having familiarity with the art will appreciate that there may be any number of variations on the particular subjects, biological sources and bases for comparing levels of at least one indicator of mitochondrial function that are useful beyond those that are expressly presented herein, and these additional uses are within the scope and spirit of the invention.

For instance, determination of levels of at least one indicator (or co-indicator) of mitochondrial function may take the form of a prognostic or a diagnostic assay performed on a skeletal muscle biopsy, on whole blood collected from a subject by routine venous blood draw, on buffy coat cells prepared from blood or on biological samples that are other cells, organs or tissue from a subject. Alternatively, in certain situations it may be desirable to construct cybrid cell lines using mitochondria from either control subjects or subjects suspected of being at risk for type 2 DM. Such cybrids may be used to determine levels of at least one indicator of mitochondrial function for diagnostic or predictive purposes, or as biological sources for screening assays to identify agents that may be suitable for treating type 2 DM based on their ability to alter the levels of at least one indicator of mitochondrial function in treated cells.

In one embodiment of this aspect of the invention, therapeutic agents or combinations of agents that are tailored to effectively treat an individual patient's particular disease may be identified by routine screening of candidate agents on cybrid cells constructed with the patient's mitochondria. In another embodiment, a method for identifying subtypes of type 2 DM is provided, for example, based on differential effects of individual candidate agents on cybrid cells constructed using mitochondria from different type 2 DM subjects.

In other embodiments, the invention provides a method of identifying an agent suitable for treating a subject suspected of being at risk for having type 2 DM by comparing the level of at least one indicator of mitochondrial function, or by comparing the level of a co-indicator of mitochondrial function and at least one non-enzyme indicator of mitochondrial function, in the presence and absence of a candidate agent, to determine the suitability of the agent for treating type 2 DM. In particularly preferred embodiments, the agent is a small molecule.

Candidate agents for use in a method of screening for a modulator of an indicator of mitochondrial function according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples, and then assayed for their ability to increase or decrease the level of at least one indicator of mitochondrial function.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCTIUS91/04666, which are hereby incorporated by reference in their entireties) or other compositions that may include small molecules as provided herein (see e.g., PCT/IJS94/08542, EP 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries may be prepared according to established procedures, and tested for their influence on an indicator of mitochondrial function, according to the present disclosure.

The present invention provides compositions and methods that are useful in pharmacogenomics, for the classification and/or stratification of a subject or patient population. In one embodiment, for example, such stratification may be achieved by identification in a subject or patient population of one or more distinct profiles of at least one indicator (or co-indicator) of mitochondrial function that correlate with type 2 DM. Such profiles may define parameters indicative of a subject's predisposition to develop type 2 DM, and may further be useful in the identification of novel subtypes of type 2 DM. In another embodiment, correlation of one or more traits in a subject with at least one indicator (or co-indicator) of mitochondrial function may be used to gauge the subject's responsiveness to, or the efficacy of, a particular therapeutic treatment. In another embodiment of the invention, measurement of the level(s) of at least one indicator (or co-indicator) of mitochondrial function in a biological sample from a subject is combined with identification of the subject's potential IGT status to determine the risk for, or presence of, type 2 DM in the subject. By using the combination of the methods for determining levels of at least one indicator of mitochondrial function as disclosed herein, and methods known in the art for determining the presence of IGT or type 2 DM (Gavin et al. Diabetes Care 22(suppl. 1):S5-S19, 1999), an enhanced ability to detect the relative risk for type 2 DM is provided by the instant invention along with other related advantages. Similarly, where levels of at least one indicator (or co-indicator) of mitochondrial function and risk for type 2 DM are correlated, the present invention provides advantageous methods for identifying agents suitable for treating type 2 DM, where such agents affect levels of at least one indicator of mitochondrial function in a biological source.

As described herein, determination of levels of at least one indicator of mitochondrial function may also be used to stratify a type 2 DM patient population (i.e., a population classified as having type 2 DM by independent criteria). Accordingly, in another preferred embodiment of the invention, determination of levels of at least one indicator of mitochondrial fumction in a biological sample from a type 2 DM subject may provide a useful correlative indicator for that subject. A type 2 DM subject so classified on the basis of levels of at least one indicator of mitochondrial function may be monitored using type 2 DM clinical parameters referred to above, such that correlation between levels of at least one indicator of mitochondrial function and any particular clinical score used to evaluate type 2 DM may be monitored. For example, stratification of a type 2 DM patient population according to levels of at least one indicator of mitochondrial function may provide a useful marker with which to correlate the efficacy of any candidate therapeutic agent being used in type 2 DM subjects.

In certain other embodiments, the invention provides a method of treating a patient having type 2 DM by administering to the patient an agent that substantially restores at least one indicator (or co-indicator) of mitochondrial function to a level found in control or normal subjects. In one embodiment the indicator of mitochondrial function is the amount of ATP produced. In another embodiment, the indicator of mitochondrial function is the amount of mtDNA present. In a most preferred embodiment, an agent that substantially restores (e.g., increases or decreases) at least one indicator of mitochondrial function to a normal level effects the return of the level of that indicator to a level found in control subjects. In another preferred embodiment, the agent that substantially restores such an indicator confers a clinically beneficial effect on the subject. In another embodiment, the agent that substantially restores the indicator promotes a statistically significant change in the level of at least one indicator (or co-indicator or co-predictor) of mitochondrial function. As noted herein, those having ordinary skill in the art can readily determine whether a change in the level of a particular indicator brings that level closer to a normal value and/or clinically benefits the subject. Thus, an agent that substantially restores at least one indicator of mitochondrial function to a normal level may include an agent capable of fully or partially restoring such level.

EXPRESSION SYSTEMS

In order to produce a gene product of interest in sufficient quantities for further embodiments of the invention, the nucleotide sequence of interest, such as a PGC or NRF, or functional equivalents thereof, is inserted into an appropriate “expression vector,” i.e., a genetic element, often capable of autonomous replication, which contains the necessary elements for the transcription and, in instances where the gene product is a protein, translation of the inserted nucleotide sequence. A genetic element that comprises an expression vector and a nucleic acid of interest in an arrangement appropriate for expression of a gene product of interest is referred to herein as an “expression construct.”

Methods which are well known to those skilled in the art can be used to prepare expression constructs containing a nucleotide sequence of interest and appropriate transcriptional and translational controls. These methods include in vitro recombinant DNA techniques, synthetic techniques and in vivo recombination or genetic recombination. Such techniques are known in the art (see, e.g., Sambrook et al., Molecular Cloning: A Laboratory Manual, Second Edition, Cold Spring Harbor Press, Plainview N.Y., 1989; Ausubel et al., eds., Short Protocols in Molecular Biology, Second Edition, John Wiley & Sons, New York N.Y., 1992).

A variety of expression vector/host systems may be utilized to contain and express a nucleotide sequence of interest. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322-based plasmids); or animal cell systems.

The “control elements” or “regulatory sequences” of these systems, which may vary in their strength and specificities, are those nontranslated regions of the vector, enhancers, promoters, and 5′ and 3′ untranslated regions, which interact with host cellular proteins to carry out transcription and, where the gene product of interest is a protein, translation. Depending on the vector system and host utilized, any number of suitable transcription and translation elements, including constitutive and inducible promoters, may be used. For example, when cloning in bacterial systems, inducible promoters, including hybrid promoters, such as lacZ promoter of the Bluescript™ phagemid (Stratagene, La Jolla, Calif.) or pSportl (Life Technologies, Inc., Rockville, Md.) and ptrp-lac hybrids and the like may be used. In insect cells, the baculovirus polyhedrin promoter may be used. Promoters and/or enhancers derived from the genomes of plant cells (e.g., heat shock, RUBISCO; and storage protein gene promoters) or from plant viruses or pathogens (e.g., viral or Agrobacterium-based promoters or leader sequences) may be cloned into the vector. In mammalian cell systems, promoters from mammalian genes or from mammalian viruses are appropriate. If it is necessary to generate a cell line that contains multiple copies of the nucleotide sequence of interest, vectors based on SV40 or EBV may be used with an appropriate selectable marker.

In bacterial systems, a number of expression vectors may be selected depending upon the use intended for expressed gene product of interest. For example, when large quantities of a protein of interest are needed for the induction of antibodies, vectors which direct high level expression of the protein of interest, or fusion proteins derived therefrom that are more readily assayed and/or purified, may be desirable.

Such vectors include, but are not limited to, Escherichia coli cloning and expression vectors such as pET (Stratagene, La Jolla, Calif.), pRSET (Invitrogen, Carlsbad, Calif.) or PGEMEX™ (Promega, Madison, Wis.) vectors, in which the sequence encoding a protein of interest is ligated downstream from a bacteriophage T7 promoter and ribosome binding site so that, when the expression construct is transformed into E. coli expressing the T7 RNA polymerase, large levels of the polypeptide of interest are produced; PGEM™ vectors (Promega), in which inserts into sequences encoding the lacZ α-peptide may be detected using colorimetric screening; and the like. For polypeptides that are relatively insoluble, it may be desirable to produce thioredoxin fusion proteins using, for example, pBAD/Thio-TOPO vectors (Invitrogen).

Plasmids such as pGEX vectors (Amersham Pharmacia Biotech, Piscataway, N.J.) may be used to express polypeptides of interest as fusion proteins. Such vectors comprise a promoter operably linked to a glutathione S-transferase (GST) gene from Schistosoma japonicum (Smith et al., 1988, Gene 67:31-40), the coding sequence of which has been modified to comprise a thrombin cleavage site-encoding nucleotide sequence immediately 5′ from a multiple cloning site. GST fusion proteins can be detected by Western blots with anti-GST or by using a colorimetric assay; the latter assay utilizes glutathione and 1-chloro-2-4-dinitrobenzene (CDNB) as substrates for GST and yields a yellow product detectable at 340 nm (Habig et al., 1974, J. Biol Chem. 249:7130-7139). GST fusion proteins produced from expression constructs derived from this expression vector can be purified by, e.g., adsorption to glutathione-agarose beads followed by elution in the presence of free glutathione. Another series of expression vectors of this type are the pBAD/His vectors (Guzman et al., J. Bact. 177:4121-4130, 1997; Invitrogen, Carlsbad, Calif.), which contains the following elements operably linked in a 5′ to 3′ orientation: the inducible, but tightly regulatable, araBAD promoter; optimized E. coli translation initiation signals; an amino terminal polyhistidine(6xHis)-encoding sequence (also referred to as a “His-tag”); an XPRESS™ epitope-encoding sequence; an enterokinase cleavage site which can be used to remove the preceding N-terminal amino acids following protein purification, if so desired; a multiple cloning site; and an in-frame termination codon. Fusion proteins made from pBAD/His expression constructs can be purified using substrates or antibodies that specifically bind to the His-tag, and assayed by Western analysis using the Anti-Xpress™ antibody. Proteins made in such systems are designed to include heparin, thrombin, enterokinase, factor XA or other protease cleavage sites so that the cloned polypeptide of interest can be released from the GST moiety by treatment with the appropriate protease.

Expression vectors derived from bacteriophage, including cosmids and phagemids, may also be used to express nucleic acids of interest in bacterial cells. Such vectors include, but are not limited to, ZAP Express™, Lambda ZAP™, and Lambda gtl 1 bacteriophage vectors, pBluescript™ phagemids, (all available from Stratagene) and the pSL 1180 Superlinker Phagemid (Amersham Pharmacia Biotech).

In yeast such as Saccharomyces cerevisiae or Pichia pastoris, a number of vectors containing constitutive or inducible promoters such as those for mating factor alpha, GAL1, TEF1, AOX1 or GAP may be used. Appropriate expression vectors include various pYES, pYD and pTEF derivatives (Invitrogen) (see, for example, Grant et al., Methods in Enzymology 153:516-544, 1987; Lundblad et al., Units 13.4 to 13.7 of Chapter 13 in: Short Protocols in Molecular Biology, 2nd Ed., Ausubel et al., eds., John Wiley & Sons, New York, N.Y., 1992, pages 13-19 to 13-33).

In cases where plant expression vectors are used, the expression of a nucleotide sequence of interest may be driven by any of a number of promoters. For example, viral promoters such as the 35S and 19S promoters of CaMV (Brisson et al., Nature 310:511-514, 1984) may be used alone or in combination with the omega leader sequence from TMV (Takamatsu et al., EMBO J. 6:307-311, 1987). Alternatively, plant promoters such as the promoter of the gene encoding the small subunit of RUBISCO (Coruzzi et al.., EMBO J. 3:1671-1680, 1984; Broglie et al., Science 224:838-843, 1984); or heat shock promoters (Winter and Sinibaldi, Results Probl. Cell. Differ. 17:85-105, 1991) may be used. These constructs can be introduced into plant cells by direct DNA transformation or pathogen-mediated transfection. For reviews of such techniques, see Gossen et al. (Curr. Opin. Biotechnol. 5:516-520, 1994), Porta and Lomonossoff (Mol. Biotechnol. 3:209-221, 1996) and Turner and Foster (Mol. Biotechnol. 3:225-36, 1995).

Another expression system which may be used to express a gene product of interest is an insect system. In one such system, Autographa californica nuclear polyhedrosis virus (AcNPV) is used as a vector to express foreign genes in Spodoptera frugiperda cells or in Trichoplusia larvae. The nucleotide sequence of interest may be cloned into a nonessential region of the virus, such as the polyhedrin gene, and placed under control of the polyhedrin promoter. Successful insertion of the sequence of interest will render the polyhedrin gene inactive and produce recombinant virus lacking coat protein. The recombinant viruses are then used to infect S. frugiperda cells or Trichoplusia larvae in which the gene product of interest is expressed (see “Piwnica-Worms, Expression of Proteins in Insect Cells Using Baculovirus Vectors,” Section II of Chapter 16 in: Short Protocols in Molecular Biology, 2nd Ed., Ausubel et al., eds., John Wiley & Sons, New York, N.Y., 1992, pages 16-32 to 16-48; Lépez-Ferber et al., Chapter 2 in: Baculovirus Expression Protocols, Methods in Molecular Biology, Vol. 39, C. R. Richardson, Ed., Humana Press, Totawa, N.J., 1995, pages 25-63). S. frugiperda cells (Sf9, Sf21 or High Five™ cells) and appropriate baculovirus transfer vectors are commercially available from, e.g., Invitrogen. Expression systems utilizing Drosophila S2 cells (also available from Invitrogen) may also be utilized.

Expression constructs for expressing nucleic acids of interest in mammalian cells are prepared in a step-wise process. First, expression cassettes that comprise a promoter (and associated regulatory sequences) operably linked to a nucleic acid of interest are constructed in bacterial plasmid-based systems; these expression cassette-comprising constructs are evaluated and optimized for their ability to produce the gene product of interest in mammalian cells that are transiently transfected therewith. Second, these expression cassettes are transferred to viral systems that produce recombinant proteins during lytic growth of the virus (e.g., SV40, BPV, EBV, adenovirus; see below) or from a virus that can stably integrate into and transduce a mammalian cellular genome (e.g., a retroviral expression construct).

With regard to the first step, commercially available “shuttle” (i.e., capable of replication in both E. coli and mammalian cells) vectors that comprise promoters that function in mammalian cells and can be operably linked to a nucleic acid of interest include, but are not limited to, SV40 late promoter expression vectors (e.g., pSVL, Amersham Pharmacia Biotech), glucocorticoid-inducible promoter expression vectors (e.g., pMSG, Amersham Pharmacia Biotech), Rous sarcoma enhancer-promoter expression vectors (e.g., pRc/RSV, Invitrogen) and CMV immediate early promoter expression vectors, including derivatives thereof having selectable markers to agents such as Neomycin, Hygromycin or ZEOCIN™ (e.g., pRc/CMV2, pCDM8, pcDNA1.1, pcDNA1.1Amp, pcDNA3.1, pcDNA3.1/Zeo and pcDNA3.1/Hygro, Invitrogen). In general, preferred shuttle vectors for nucleic acids of interest are those having selectable markers (for ease of isolation and maintenance of transformed cells) and inducible, and thus regulatable, promoters as overexpression of a gene product of interest may have toxic effects.

Methods for transfecting mammalian cells are known in the art (see, Kingston et al., “Transfection of DNA into Eukaryotic Cells,” Section 1 of Chapter 9 in: Short Protocols in Molecular Biology, 2nd Ed., Ausubel et al., eds., John Wiley & Sons, New York, N.Y., 1992, pages 9-3 to 9-16). A control plasmid, such as pCH110 (Pharmacia), may be cotransfected with the expression construct being examined so that levels of the gene product of interest can be normalized to a gene product expressed from the control plasmid. Preferred expression cassettes, consisting essentially of a promoter and associated regulatory sequences operably linked to a nucleic acid of interest, are identified by the ability of cells transiently transformed with a vector comprising a given expression cassette to express high levels of the gene product of interest, or a fusion protein derived therefrom, when induced to do so. Expression may be monitored by Northern or Western analysis or, in the case of fusion proteins, by a reporter moiety such as an enzyme or epitope. Effective expression cassettes are then incorporated into viral expression vectors.

Nucleic acids, preferably DNA, comprising preferred expression cassettes are isolated from the transient expression constructs in which they were prepared, characterized and optimized. A preferred method of isolating such expression cassettes is by amplification by PCR, although other methods (e.g., digestion with appropriate restriction enzymes) can be used. Preferred expression cassettes are introduced into viral expression vectors, preferably retroviral expression vectors, in the following manner.

A DNA molecule comprising a preferred expression cassette is introduced into a retroviral transfer vector by ligation. Two types of retroviral transfer vectors are known in the art: replication-incompetent and replication-competent. Replication-incompetent vectors lack viral genes necessary to produce infectious particles but retain cis-acting viral sequences necessary for viral transmission. Such cis-acting sequences include the ψ packaging sequence, signals for reverse transcription and integration, and viral promoter, enhancer, polyadenylation and other regulatory sequences. Replication-competent vectors retain all these elements as well as genes encoding virion structural proteins (typically, those encoded by genes designated gag, pol and env) and can thus infectious particles. In contrast, these functions are supplied in trans to replication-incompetent vectors in a packaging cell line, i.e, a cell line that produces mRNAs encoding gag, pol and env genes but lacking the ψ packaging sequence. See, generally, Cepko, Unit 9.10 of Chapter 9 in: Short Protocols in Molecular Biology, 2nd Ed., Ausubel et al., eds., John Wiley & Sons, New York, N.Y., 1992, pages 9-30 to 9-35.

A retroviral construct comprising an expression cassette comprising a nucleic acid of interest produces RNA molecules comprising the cassette sequences and the ψ packaging sequence. These RNA molecules correspond to viral genomes that are encapsidated by viral structural proteins in an appropriate cell line (by “appropriate” it is meant that, for example, a packaging cell line must be used for constructs based on replication-incompetent retroviral vectors). Infectious viral particles are then produced, and released into the culture supernatant, by budding from the cellular membrane. The infectious particles, which comprise a viral RNA genome that includes the expression cassette for the gene product of interest, are prepared and concentrated according to known methods. It may be desirable to monitor undesirable helper virus, i.e., viral particles which do not comprise the expression cassette for the gene product of interest. See, generally, Cepko, Units 9.11, 9.12 and 9.13 of Chapter 9 in: Short Protocols in Molecular Biology, 2nd Ed., Ausubel et al., eds., John Wiley & Sons, New York, N.Y., 1992, pages 9-36 to 9-45.

Viral particles comprising an expression cassette for the gene product of interest are used to infect in vitro (e.g., cultured cells) or in vivo (e.g., cells of a rodent, or of an avian species, which are part of a whole animal). Tissue explants or cultured embryos may also be infected according to methods known in the art. See, generally, Cepko, Unit 9.14 of Chapter 9 in: Short Protocols in Molecular Biology, 2nd Ed., Ausubel et al., eds., John Wiley & Sons, New York, N.Y., 1992, pages 9-45 to 9-48. Regardless of the type of cell used, production of the gene product of interest is directed by the recombinant viral genome.

In eukaryotic expression systems, host cells may be chosen for its ability to modulate the expression of the inserted sequences or, when the gene product of interest is a protein, to process the protein of interest in the desired fashion. Such modifications of proteins include, but are not limited to, acetylation, carboxylation, glycosylation, phosphorylation, lipidation and acylation. Post-translational processing which cleaves a “prepro” form of the protein of interest may also be important for its correct intracellular localization, folding and/or function. Different host cells such as CHO, HeLa, MDCK, HEK293, W138, etc. have specific cellular machinery and characteristic mechanisms for such post-translational activities and may be chosen to ensure the correct modification and processing of a protein of interest.

Expression systems of the invention also include the few systems in which a nucleic acid of interest is expressed from an organellar genome. Means for the genetic manipulation of the mitochondrial genome of Saccharomyces cerevisiae (Steele et al., Proc. Natl. Acad. Sci. U.S.A. 93:5253-5257, 1996) and systems for the genetic manipulation of plant chlorplasts (U.S. Pat. No. 5,693,507; Daniell et al., Nature Biotechnology 16:345-348, 1998) have been described. Naturally, nucleic acids that encode polypeptide sequences may have to be altered in organellar expression systems in order to reflect the differences in the genetic codes of organelles (see, e.g., Table 1).

NUCLEIC ACIDS AND NUCLEOTIDE SEQUENCES

Once a nucleic of interest has been identified, it can be used to generate other useful nucleic acids having related sequences, including without limitation deoxyribonucleic acids (DNA). In a preferred embodiment, an RNA of interest is used to generate a cDNA molecule that can be used to detect nucleic acids having the sequence of interest, or to produce a polypeptide encoded by the sequence of the RNA of interest.

For example, it is known in the art to isolate mRNAs of interest and have them reverse-transcribed. Reverse transcription is a process by which a reverse complementary DNA (cDNA) is produced from an RNA molecule which acts as a template. The RNA portion of the resultant (RNA:DNA) hybrid may then be displaced or enzymatically degraded, after which the single-stranded DNA (ssDNA) is used as a template for one or more rounds of DNA polymerization, the product of which is a double-stranded DNA (dsDNA) molecule. The dsDNA molecule includes the sequence of the RNA of interest (except that uridine residues in the RNA are replaced by thymidine residues in the DNA). The nucleotide sequence of the dsDNA is then determined and analyzed; additionally or alternatively, the dsDNA is cloned, i.e., incorporated into a vector DNA that is capable of replication in an appropriate host cell. If the dsDNA molecule includes a sequence that encodes a polypeptide, a preferred vector is an expression vector.

A DNA molecule prepared according to the methods of the invention can be a full-length cDNA, i.e., one comprising a nucleotide sequence that encodes an entire protein. At a minimum, a full-length cDNA will encompass a “start” (translation initiating) codon, a “stop” (translation terminating) codon, and all the polypeptide-encoding sequences in-between.

Alternatively, a DNA molecule prepared according to the methods of the invention can be an Expressed Sequence Tag (EST), i.e., one which does not comprise a complete full length cDNA but which does comprise a nucleotide sequence that is a portion of an full length cDNA or of a mRNA comprising a full length cDNA. An EST is useful in and of itself as, e.g., a probe in methods for detecting a mRNA of interest. Because a full-length cDNA is required for, e.g., recombinant DNA expression of a protein encoded by a mRNA interest, it may also be desirable to use an EST as a tool to isolate a full-length cDNA according to a variety of methods. For example, a nucleic acid comprising an EST sequence of interest can be labeled and used to probe preparations of cellular DNA, cDNA or RNA for hybridizing sequences, and such hybridizing sequences can be isolated, amplified and cloned according to known methods. As another example, the sequence of an EST can be used to prepare primers for inverse PCR, a process by which sequences flanking an EST of interest can be determined (see, e.g., Benkel and Fong, Genet. Anal. 13:123-127, 1996; Silverman, Methods Mol. Biol. 54:145-155, 1996; Pang and Knecht, BioTechniques 22:1046-1048, 1997; Huang, Methods MoL Biol. 69:89-96, 1997; Huang, Methods Mol. BioL 67:287-294, 1997; and Offringa and van der Lee, Methods MoL BioL 49:181-195, 1996; all of which are hereby incorporated by reference).

In methods of cloning full-length cDNAs from ESTs, and as a useful method in its own right, it is desirable to screen mRNA or cDNA libraries prepared from various cells and tissues in order to identify cells and tissues that express relatively high levels of a nucleic acid of interest. For example, a nucleic acid of interest can be used to examine tissue- or temporal-specific patterns of expression of a nucleic acid of interest in a variety of methods known in the art. The nucleic acid of interest can be detectably labeled and used to probe (i) an immobilized collection of mRNA molecules (e.g., RNA Master Blots™ or Multiple Tissue Northern, MTN™, Blots from Clontech) or (ii) a cDNA library (prepared according to methods known in the art or available from, e.g., Clontech or from depositories such as the American Type Culture Collection, ATCC, Manassas, Va.). Alternatively or additionally, a sequence of interest can be used to design specific PCR primers that can be used in amplification reactions in 96-well plates wherein each well comprises first strand cDNAs from a particular tissue (e.g., the Rapid-Scan™ gene expression panel from OriGene Technologies, Inc., Rockville, Md.). In this embodiment, automated, semi-automated or robotic means may be used to carry out such assays.

Mammalian tissues that may be examined include but are not limited to brain (including, by way of example but not limitation, whole brain and subsections thereof, e.g., amygdala, caudate nucleus, cerebellum, cerebral cortex, frontal lobe, hippocampus, medulla oblongata, occipital lobe, putamen, substantia nigra, temporal lobe, thalamus, acumens, subthalamic nucleus, inferio temporal cortex, medial frontal cortex, occipital pole), heart, kidney, spleen, liver, colon, lung, small intestine, stomach, skeletal muscle, smooth muscle, testis, uterus, bladder, lymph nodes, spinal cord, dorsal root ganglia, trachea, bone marrow, placenta, salivary glands, thyroid glands, thymus, adrenal glands, pancreas, ovary, uterus, prostate, skin, bone marrow, pancreas or portions thereof such as beta cells, fetal brain and fetal liver.

In order to identify tissues or cells from which a cDNA corresponding to an EST of interest can optimally be prepared, mRNA or cDNA libraries or arrays derived from the organism from which the EST of interest was isolated are probed. Tissues or cells having a high level of expression of the nucleic acid of interest are preferably used as sources for full-length nucleic acids, i. e., nucleic acids containing all the genetic information required to express a complete gene product of interest. The full-length nucleic acids are used, e.g., to express the gene product (i.e., RNA or protein) of interest or to prepare manipulated cells or transgenic animals in which the level of expression or activity, or tissue- or temporal-specific patterns of expression, of the gene product of interest is altered relative to the wildtype condition.

Another utility of ESTs and full-length cDNAs is to search in silico for corresponding protein sequences, in order to identify proteins of interest encoded thereby and to prepare antibodies thereto. For example, the nucleotide sequence of an EST or cDNA of interest is translated in silico in all six potential reading frames (three reading frames on each strand of a dsDNA), and the resulting amino acid sequences are used as probes to search protein databases for a match to a portion of a protein having a known amino acid sequence. In the case of mitochondrial proteins, it is desirable to perform such in silico translations using both the “universal” genetic code and the somewhat different genetic code utilized in mitochondria (TABLE 1), as different amino acid sequences will result in each case.

TABLE 1
Differences Between the “Universal” and Mitochondrial Genetic Codes

| Cod- | “Universal” | Yeast Mitochondrial | Mammalian Mitochondrial |
|---|---|---|---|
| on | Genetic Code | Genetic Code | Genetic Code |
| AGA | Arg | Arg | (stop) |
| AGG | Arg | Arg | (stop) |
| AUA | Ile | Met | Met |
| CUA | Leu | Thr | Leu |
| UGA | (stop) | Trp | Trp |

Nucleic acids having or comprising a sequence of interest can be prepared by a variety of methods known in the art. For example, such nucleic acids can be made using molecular biology or synthetic techniques (Sambrook et al., Molecular Cloning, A Laboratory Manual, Cold Spring Harbor Press (1989)). Many equivalent bases, both naturally occurring and synthetic, in nucleotide sequences are known in the art. For example, thymine (T) residues in DNA are transcribed into uracil (U) residues in RNA molecules but, because both T and U specifically pair with adenine (A) residues, these changes do not impact hybridization specificity. Nucleic acids comprising such equivalent substitutions are within the scope of the disclosure. In addition, nucleic acids of the invention may have one or more non-nucleotide moieties. These non-nucleotides and their use in ribozymes are described in U.S. Pat. No. 5,891,683 and includes polyethers, polyamines, polyamides, polyhydrocarbons and abasic nucleotides.

As another example, such nucleic acids can be oligonucleotides, including oligodeoxyribonucleotides and oligoribonucleotides synthesized in vitro by, for example, the phosphotriester, phosphoramidite or H-phosphanate methodologies (see, respectively, Christodoulou, “Oligonucleotide Synthesis: Phosphotriester Approach,” Chapter 2 In: Protocols for Oligonucleotides and Analogs: Synthesis and Properties, Agrawal, ed., Methods in Molecular Biology Vol. 20, Humana Press, Totowa, N.J. (1993); Beaucage, “Oligodeoxyribonucleotide Synthesis: Phosphoramidite Approach,” Chapter 3, Id.; and Froehler, “Oligodeoxynucleotide Synthesis: H-phosphonate Approach,” Chapter 4, Id., all of which are hereby incorporated by reference).

The length of a nucleic acid according to the present invention can be chosen by one skilled in the art depending on the particular purpose for which the nucleic acid is intended. For PCR primers and antisense oligonucleotides, the length of the nucleic acid is preferably from about 10 to about 100 base nucleotides (nt), more preferably from about 12 nt to about 60 nt, and most preferably from about 15 nt to about 30 nt. For ribozymes, the length of the nucleic acid is preferably from about 20 nt to about 200 nt, more preferably from about 30 nt to about 100 nt, and most preferably from about 40 nt to about 80 nt. For probes, the length of the nucleic acid is preferably from about 10 nt to about 5,000 nt, more preferably from about 15 to about 1,000 nt, and most preferably from about 20 nt to about 500 nt.

Appropriate chemical modifications to nucleic acids of the invention are also readily chosen by one skilled in the art. Such modifications may include, for example, means by which the nucleic acid is detectably labeled for use as a probe. Typical detectable labels include radioactive moieties and reporter groups, e.g., enzymes and fluorescent or luminescent moieties. Other chemical modifications appropriate for particular uses, such as antisense applications, as explained herein.

Detectably labeled nucleic acids are preferred for diagnostic, prognostic and pharmacogenetic methods of the invention. Whether labeled or unlabeled, nucleic acids of the invention can be provided in kit form, e.g., in a single or separate container, along with other reagents, buffers, enzymes or materials to be used in practicing at least one method of the invention. The kit can be provided in a container that can optionally include instructions or software for performing a method of the invention. Such instructions or software can be provided in any language or human- or machine-readable format.

DETECTING NUCLEIC ACIDS, INCLUDING DIFFERENTIALLYEXPRESSED NUCLEIC ACIDS

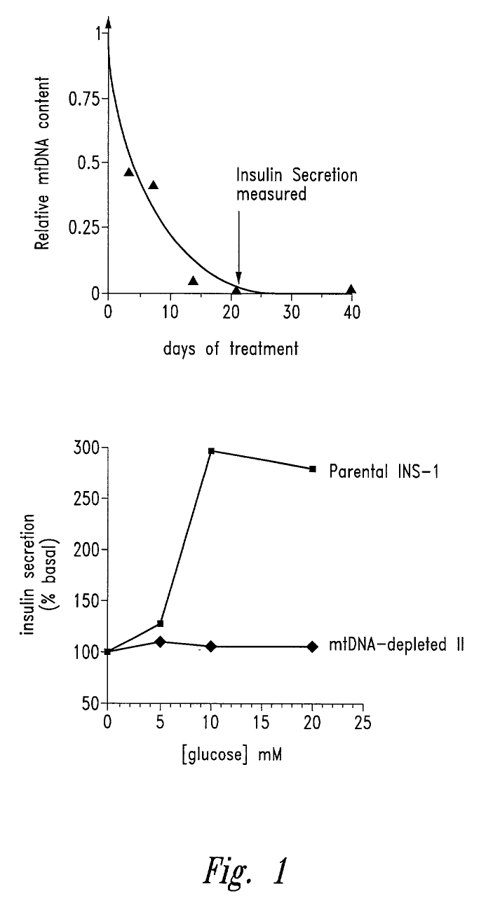

A variety of methods and means for detecting nucleic acids, including differentially expressed nucleic acids, may be used in the methods of the invention. Methods and means include, without limitation, the following methodologies. It should be noted that, regardless of which method is used to identify candidate differentially expressed genes, a second independent method should be used to verify the results obtained from the first method. Preferably, in the present invention, cells that do not express NRF, PGC or NRF and PGC are used as a first cell and cells that express NRF, PGC or NRF and PGC are used as the second cell such that differential display of the first cell and the second cell is determined. In the present invention, the first cell and the second cell can be the same cell, however, the second cell has been induced to express NRF, PGC or NRF and PGC by an appropriate inducer, such as tetracyclene, in a construct such as that described in FIG. 1.

Subtractive Hybridization: In a typical procedure for applying the technique of subtraction hybridization (Hedrick et al., Nature 308:149-153, 1984) to investigate differences in the expression of genes of a certain sample of test or target cells, e.g. from tumor tissues or tissues in a disease state, such as tissues affected by diabetes, as compared with the expression of genes of a sample of reference cells, e.g. cells from corresponding normal tissue, total cell mRNA is extracted (using any preferred method) from both samples of cells. The mRNA in the extract from the test or target cells is then used in a conventional manner to synthesize corresponding single stranded cDNA using an appropriate primer and a reverse transcriptase in the presence of the necessary deoxynucleoside triphosphates, and the template mRNA is subsequently degraded by alkaline hydrolysis or RNase H to leave only the single stranded CDNA. The single stranded cDNA thus derived from the mRNA expressed by the test or target cells is then mixed under hybridizing conditions with an excess quantity of the mRNA extract from the reference (normal) cells; this mRNA is generally termed the subtraction hybridization “driver” since it is this mRNA or other single stranded nucleic acid present in excess which “drives” the subtraction process. As a result, cDNA strands having common complementary sequences anneal with the mRNA strands to form mRNA/cDNA duplexes and are thus subtracted from the single stranded species present. The only single stranded DNA remaining is then the unique cDNA that is derived specifically from the MRNA produced by genes which are expressed solely by the test or target cells. Alternatively, the reference cells may be used as a source of single-stranded DNA, and the test or target cells may be used as a source of driver RNA. In this case the remaining single-stranded DNA is derived from mRNA produced by genes expressed in the reference cells but not in the target cells.

To complete the subtraction process, it is generally desirable to physically to separate out the common mRNA/cDNA duplexes, using for example hydroxyapatite (HAP) or (strept)avidin-biotin in a chromatographic separation method. One or more repeat rounds of the subtraction hybridization may be carried out to improve the degree of removal of commonly expressed sequences, although other means may be employed (see, e.g., U.S. Pat. No. 5,589,339). It is generally desirable to clone the sequences isolated by subtractive hybridization, such that they may be amplified and to facilitate identification. The single-stranded CDNA may be converted to double-stranded DNA by methods or means know in the art. For example, multiple copies of a single nucleotide, for example deoxycytidine may be added, onto the 3′ end of the single-stranded DNA molecules using an enzyme such as terminal transferase, and then an oligonucleotide of complementary sequence, e.g. poly G to prime synthesis of the complementary strand using any of a number of commercially available DNA polymerases can be used. The CDNA sequences obtained from subtractive hybridization may be used to produce labeled probes that may perhaps then be used for detecting or identifying corresponding cloned copies in a CDNA clone colony or cDNA library (labeling of such probes is frequently introduced by using labeled deoxynucleoside triphosphates in synthesis of the cDNA),

High Density Arrays: Multiple sample nucleic acid hybridization analysis can be carried out on micro-formatted multiplex or matrix devices (e.g., DNA or RNA chips, filters and microarrays) (see, e.g., Bains, Bio/Technology 10:757-758, 1992). These hybridization formats are micro-scale versions of the conventional “fdot blot” and “sandwich” hybridization systems. In these methods, specific DNA sequences are typically attached to, or synthesized on, very small specific areas of a solid support, allowing large numbers of different DNA sequences to be placed in a small area. The high density arrays comprise target elements, i.e., target nucleic acid molecules bound to a solid support. The nucleic acids for both the target elements and the probes may be, for example, RNA, DNA, or CDNA. In one type of array, target elements comprising nucleic acid elements that are short synthetic oligonucleotides derived from mRNA, cDNA or EST sequences are used to carry out serial analysis of gene expression (SAGE; U.S. Pat. No. 5,866,330).

In methods for comparing two nucleic acid collections, nucleic acid molecules in the test and control collections (which may be, e.g., mRNA preparations from a diseased and undiseased human) are detectably labeled. The first and second labeled probes thus formed are each contacted to an identical high density array comprising a plurality of target elements under conditions such that nucleic acid hybridization to the target elements can occur.

After contacting the probes to the target elements the amount of binding to each target element in each of the two arrays is measured, and the binding ratio (i.e., amount bound in the disease sample/amount bound in the control sample) is determined for each target element. A binding ratio >1 indicates that nucleic acids hybridizing to the particular target element are “up-regulated” in the nucleic acid collection prepared from the diseased patient relative to the nucleic acid prepared from the control individual, whereas a binding ratio <1 indicates that nucleic acids hybridizing to the particular target element are “down-regulated” in the diseased patient.

High density cDNA arrays that may be used in the invention include but are not limited to GeneChip™ arrays comprising synthetic oligonucleotides (Affymetrix, Inc., Santa Clara, Calif.); GeneFilters™ yeast or human cDNA arrays (Research Genetics, Huntsville, Ala.); ATLAS™ cDNA arrays (Clontech); and GEM™ and Gene Display Arrays (GDA) cDNA arrays (Genome Systems, Inc., St. Louis, Mo.). Furthermore, one method for building a microarrayer (a machine that produces microarrays) is available on-line at http://cmgm.stanford.edu/pbrown/mguide/index.html.

One type of high density array uses electronic hybridization, i.e., a method that directs sample DNA molecules to, and concentrates them at, test sites on a microchip that can be electronically activated by a positive charge. Because DNA molecules in solution have strong negative charges, they are attracted to activated sites. The electronic hybridization of sample DNA molecules at each test site promotes rapid hybridization of the sample DNAs with the nucleic acids of the target elements. Materials for electronic hybridization are available from Nanogen (San Diego, Calif.) and the method is described in U.S. Pat. No. 5,849,486.

Differential Display. To investigate differences in the expression of genes of a certain sample of test or target cells, such as tissues affected by diabetes, as compared with the expression of genes of a sample of reference cells, e.g. cells from corresponding normal brain tissue, the RNA may be reverse transcribed and amplified with specific primer sets, and the resulting amplification products from the two samples compared (Hipfel R, et al. (1998) J. Biochem Biophys. Methods 37: 131-135; Bosch T C and Lohmann J U (1998) Mthods Mol Biol 86: 153-160). Total cell RNA is extracted (using any preferred method) from both samples of cells. The RNA from both samples is reverse transcribed using a set of twelve primers containing a sequences of poly (T) terminating in one of either AA, AC, AG, AT, CA, CC, CG, CT, GA,GC, GG, or GT. The single stranded cDNAs of the resulting cDNA/mRNA hybrids are then amplified in separate reactions, with each reaction using one of the set of twelve “3′” primers used in the reverse transcription reaction and one of a set of “5′” primers. Typically a set of about twenty 5′ primers is used, each with a different arbitrary sequence. The resulting amplification products are labeled, preferably by using primers which have incorporated a fluorescent dye, but other labeling methods and other labels may be used, and electrophoresed such as on gels. The products resulting from reverse transcription and amplification of RNA from two different samples with the same primer sets are compared. Bands which are overexpressed or underexpressed in one sample when compared with another sample may be excised from the gel, reamplified, cloned, and sequenced to identify genes with different levels of expression in the two samples.

GENETIC MODULATION OFNUCLEICACIDSAND GENE PRODUCTS

Various antisense-based methodologies may be used to modulate (reduce or eliminate) the expression of a nucleic acid of interest, and the corresponding gene product, in organelles, cells, tissues, organs and organisms. Such antisense modulation may be used to validate the role of a gene of interest in a disease or disorder or, when the causes or symptoms of a disease or disorder result from the over-expression of a nucleic acid of interest, as therapeutic agents. In the case of the present invention, the expression of NRF or PGC, or both, can be increased by interfering with the transcription or translation of inhibitors of NRF or PGC transcription or translation. Alternatively, the expression of NRF or PGC, or both, can be decreased by interfering with the transcription or translation of activators or NRF or PGC transcription or translation or by interfering with the transcription or translation of NRF or PGC themselves.

The term “antisense” refers to nucleic acids that comprise one or more sequences that are the reverse complement of the “sense” strand of a gene, i.e., the strand that is transcribed and, in the case of protein-encoding sequences, translated. Because antisense nucleic acids bind with high specificity to their targeted nucleic acids, selectivity is high and toxic side effects resulting from misdirection of the compounds can be minimal.

In general, antisense compositions are of two types: (i) synthetic antisense oligonucleotides, including enzymatic ones, e.g., ribozymes; and (ii) antisense expression constructs. One skilled in the art will be able to utilize either modality as is appropriate to the given situation.

Synthetic antisense oligonucleotides are prepared from the reverse complement of a nucleic acid of interest. An antisense oligonucleotide consists of nucleic acid sequences corresponding to the reverse complement of a differentially expressed RNA. When introduced into cells expressing the RNA of interest, the antisense oligonucleotides specifically bind to the RNA molecules and interfere with their finction by preventing secondary structures from forming or blocking the binding of regulatory or RNA-stabilizing factors. In addition, in the case of protein-encoding RNA species, oligonucleotides can inhibit RNA splicing, polyadenylation or protein translation, thus limiting or preventing the amount of protein made from such mRNAs. Additionally or alternatively, such oligoncuelotides can bind to double-stranded DNA molecules and form triplexes therewith, and thus interfere with the transcription of such sequences.

In instances where it is desired to target antisense oligonucleotides to RNAs produced from organellar genomes, peptide nucleic acids (PNAs) are preferred synthetic oligonucleotides. In PNAs, the sugar-phosphate backbone of biological nucleic acids has been replaced with a polypeptide-like chain. Targeting sequences that direct proteins to organelles can be conjugated to the backbone of antisense PNAs, with the result being that such conjugates are preferentially delivered to the targeted organelle (see, for example, published PCT applications WO 97/41150 and WO 99/05302.

Antisense oligonucleotides may be inherently enzymatic in nature, that is, capable of degrading the RNA molecule towards which they are targeted; such molecules are generally referred to as “ribozymes.” A variety of increasingly short synthetic ribozyme frameworks that can be modified to comprise a nucleic acid sequence of interest have been described (Couture and Stinchcomb, Trends Genet. 12:510-515, 1996), including but not limited to hairpin ribozymes (Hampel, Prog. Nucleic Acid Res. Mol. Biol. 58:1-39, 1998), hammerhead ribozymes (Birikh et al., Eur. J. Biochem. 245:1-16, 1997) and minizymes (Kuwabara et al., Nature Biotechnology 16:961-965, 1998).

In the case of non-catalytic antisense nucleic acids and ribozymes antisense modulation of gene expression in a cell can also be achieved by expression constructs that direct the transcription of the reverse complement of a nucleotide sequence of interest in vivo. For example, in order to express non-catalytic antisense transcripts in mammalian or plant cells, all that may be required is the “flipping” (i.e., reversing the orientation) of a nucleic acid of interest that has been cloned into a mammalian or plant expression vector, respectively. It is not necessary to maintain the proper relationship of elements such as translation signals and the like, as the minimum requirement for an antisense expression construct of this type is a promoter operably linked to the reverse complement of a nucleic acid of interest. It is also possible to design expression constructs that express ribozymes in cells. Antisense and ribozyme expression constructs are also used to produce transgenic animals in which the level of expression of a gene of interest can be modulated in a temporal- or tissue-specific manner (see Sokol and Murray, Transgenic Res. 5:363-371, 1996, for a review).

Nucleic acid sequences derived according to the present invention may also be used to design “RNA decoys,” i.e., short RNA molecules corresponding to cis-acting regulatory sequences that bind trans-acting regulatory factors. When overexpressed in a cell or administered in excess thereto, such RNA decoys competitively inhibit the binding and thus action of the trans-acting regulatory factors, and thus limit or prevent the ability of such factors to carry out processes that stabilize (or destabilize) the RNA of interest, or enhance (or decrease) the polyadenylation, splicing nuclear transport, or translation of the RNA (Sullenger et al., J. Virol. 65:6811-6816, 1991). Expression of the RNA of interest may thus be either enhanced or decreased for therapeutic purposes.

POLYPEPTIDES AND PROTEINS

The nucleic acids of interest identified according to the methods of the invention may encode amino acid sequences. Such amino acid sequences may correspond to a full-length protein or to a polypeptide portion thereof. The present invention also includes polypeptides that are derivatives of PGC or NRF, or polypeptides that have at least one activity of PGC or NRF.

In instances wherein a full-length protein is encoded by a nucleic acid of interest, the protein may be a known protein that is commercially available or one to which antibodies are known and can be used to isolate the protein from appropriate biological samples. If a full-length protein of the invention has not previously been described, it may be produced via recombinant DNA methodologies for example, using the expression systems described previously, or prepared from biological samples using known biochemical techniques. Short (i.e., having less than about 30 amino acids) polypeptides that are encoded by short (i.e., having less than about 100 nucleotides) nucleic acids of the invention or derived from the amino acid sequences encoded by longer nucleic acids or from full-length proteins can be synthesized in vitro by methods known in the art. Fusion proteins comprising amino acid sequences of interest may also be prepared and are included within the scope of the polypeptides and proteins of the invention.

Regardless of the means by which they are prepared, the polypeptides and proteins of the invention have a variety of applications. They may be used to generate antibodies or to screen for ligands that may serve as therapeutic agents, or may themselves be used as therapeutic agents. Full-length proteins of the invention may have the activity of the wildtype protein and may thus be used to treat conditions resulting from a loss of such activity. Polypeptides of the invention may also have such activities, or may competitively inhibit a protein of interest in vivo by binding a ligand of the protein. If the ligand is an activator of the protein, such polypeptides may be used to treat conditions resulting from the over-expression or over-activation of the protein in vivo. If the ligand is a toxin or activator of cell death (apoptosis or necrosis), administration of a protein or polypeptide that binds such a ligand to a patient in need thereof will have the beneficial effect of competitively inhibiting the action of the toxin or cell death activator.

ANTIBODIES

Antibodies to a protein or polypeptide of interest are prepared according to a variety of methods known in the art. In particular, antibodies that bind with NRF, PGC or a label sequence, such as FLAG, can be used to detect NRF or PGC or a label sequence, particularly in a cell, using labeled antibodies that bind with such polypeptides. In general, such antibodies may be polyclonal, monoclonal or monospecific antibodies. Primary antibodies of the invention bind specifically to a particular protein or polypeptide of interest and are thus used in assays to detect and quantitate such proteins and polypeptides. The invention also includes active fragments or active portions that exhibit the binding specificity or the substantial binding specificity of the intact antibody they were derived from. In such assays, generally referred to in the art as immunoassays, a primary antibody of the invention is detectably labeled or is specifically recognized and monitored by a detectably labeled secondary antibody or a combination of a secondary antibody and a tertiary molecule (which may also be an antibody) that is detectably labeled. Regardless of the specific format, the primary antibody of the invention provides a means by which a protein or polypeptide of interest is specifically bound and subsequently detected. One preferred assay format is the Enzyme-Linked Immunosorbent Assay (ELISA) format.

A nucleic acid of interest may encode a known protein or a portion thereof, or a polypeptide sequence that is homologous to a known protein. In such instances, antisera to the known protein, or the known protein itself, may be commercially available. In the latter instance, or when the nucleic acid of interest can be used to produce a protein of interest (or a polypeptide portion thereof greater than about 30 amino acids in length) via recombinant DNA expression techniques, the known or recombinantly-produced protein can be used to immunize a mammal of choice (e.g., a rabbit, mouse or rat) in order to produce antisera from which polyclonal antibodies can be prepared (see, e.g., Cooper and Paterson, Units 11.12 and 11.13 in Chapter 11 in: Short Protocols in Molecular Biology, 2nd Ed., Ausubel et al., eds., John Wiley & Sons, New York, N.Y., 1992, pages 11-37to 11-41).

In the event that a nucleic acid sequence of interest encodes a polypeptide sequence for which no complete protein (or homolog thereof) is known, is too short to encode more than about 30 amino acids (i.e., the nucleic acid of interest is less than about 100 nucleotides in length), or encodes more than one polypeptide sequence of potential interest, such candidate amino acid sequences can be used to synthesize one or more polypeptide molecules, each of which has a defined amino acid sequence. Such synthetic polypeptides can then be used to immunize animals (e.g., rabbits) according to methods known in the art (Collawn and Paterson, Units 11.14 and 11.15 in Chapter 11 in: Short Protocols in Molecular Biology, 2nd Ed., Ausubel et al., eds., John Wiley & Sons, New York, N.Y., 1992, pages 11-42 to 11-46; Cooper and Paterson, Units 11.12 and 11.13 in Chapter 11 in: Short Protocols in Molecular Biology, 2nd Ed., Ausubel et al., eds., John Wiley & Sons, New York, N.Y., 1992, pages 11-37 to 11-41). The resulting antisera, sometimes referred to as “monospecific,” may then be used to probe cells from which the nucleic acid of interest was isolated. A positive response to a given antiserum indicates that the candidate reading frame from which the synthetic polypeptide used to raise the antiserum was derived is a reading frame used to encode at least one protein in the cell(s) so examined. Moreover, such an antiserum can be used to identify proteins of interest in the cells from which the nucleic acid of interest was isolated.

Because of their high degree of specificity and homogeneity, monoclonal antibodies are often the preferred type of antibody for a variety of applications. Methods for producing and preparing monoclonal antibodies are known in the art (see, e.g., Fuller et al., Units 11.4 to 11.11 in Chapter 11 in: Short Protocols in Molecular Biology, 2nd Ed., Ausubel et al., eds., John Wiley & Sons, New York, N.Y., 1992, pages 11-22 to 11-36). Murine monoclonal antibodies may be “humanized” to reduce their antigenicity in humans and used as therapeutic agents (see, e.g., Güssow and Seemann, Methods in Enzymology 203:99-121, 1991; Vaughan et al., Nature Biotechnology 16:535-539, 1998).

Antibodies to proteins and polypeptides of interest are used to detect such proteins and polypeptides in a variety of assay formats. Such immunoassays may useful in diagnostic, prognostic or phannacogenetic methods of the invention, or in methods in which various cell types, tissues or organs are probed for the presence of a protein of interest. Monoclonal antibodies are generally preferred for such methods due to their high degree of specificity and homogeneity.

DIAGNOSTIC, PROGNOSTIC AND PHARMACOGENETIC METHODS

Assays for or utilizing one or more of the antibodies, polypeptides and proteins, ligands therefor and nucleic acids of the invention are used in diagnostic, prognostic and pharmacogenetic methods of the invention. The term “diagnostic” refers to assays that provide results which can be used by one skilled in the art, typically in combination with results from other assays, to determine if an individual is suffering from a disease or disorder of interest such as diabetes, including type I and type II, whereas the term “prognostic” refers to the use of such assays to evaluate the response of an individual having such a disease or disorder to therapeutic or prophylactic treatment. The term “pharmacogenetic” refers to the use of assays to predict which individual patients in a group will best respond to a particular therapeutic or prophylactic composition or treatment.

The terms “disease” and “disorder” refers to diabetes, either type I or type II.

In diagnostic and prognostic applications of the invention, samples from individuals are assayed with regard to the relative or absolute amounts of a “marker,” i.e., a nucleic acid or protein of interest, or an endogenous ligand of or antibody to a nucleic acid or protein of interest. An increased or decreased level of a marker relative to control levels indicates that the individual from which the sample was taken has, has had, or is likely to develop the disease or disorder of interest. The term “control level” refers to the level of marker present in samples taken from one or more individuals known to not have the disease or disorder of interest, or to the level of marker present in a sample taken from the individual in question before or after the diagnostic sample. Additionally or alternatively, a number of individuals known to not have the disease or disorder of interest are tested for levels of the marker, and an absolute amount or concentration corresponding to a normal level of the marker is established; in this embodiment, affected individuals are identified as those having a level of marker that is significantly lower or higher than the normal value. In addition, nucleic acids of the invention may be used to screen for single nucleotide polymorphisms (SNPs) and other mutations such as gene deletions or insertions, by hybridization methods (Sapolsky R J et al. Genet. Anal. (1999) 14: 187-192), or other methods as they are known or later developed in the art.

In pharmacogenetic applications of the invention, patients suffering from a disease or disorder of interest are stratified with regards to desirable or undesirable responses to a potential treatment using one or more assays of the invention. A therapeutic composition and/or treatment known to be more effective, or which produces fewer side-effects, in some patients as compared to others is administered a group of patients suffering from a disease or disorder of interest. A method of identifying which patients having the disease are more likely to respond to a therapeutic composition and/or treatment comprises providing samples from a group of patients having said disease; measuring the amount or molecular attribute of a protein or polypeptide of interest, or of a nucleic acid of interest, or a ligand therefor or antibody thereto, or any combination thereof present in said samples; providing the therapeutic composition and/or treatment to the patients; measuring the degree, frequency, rate or extent of responses of the patients to the therapeutic composition and/or treatment; and determining if a correlation exists between the amount or molecular attributes of a nucleic acid of interest, or the amount or molecular attributes of a protein or polypeptide of interest, or a ligand therefor or antibody thereto present in said samples and the degree, frequency, rate or extent of such responses.

The resulting correlations are used to stratify patients in the following manner. If such a correlation is a positive correlation, the presence of such correlation indicates that patients yielding samples having an increased or decreased amount, relative to the established normal range, of the protein or polypeptide of interest, or the ligand or antibodies therefor, or nucleic acid molecules, or an increase or decreased amount, relative to the established normal range, of the nucleic acid of interest, are more likely to respond to said treatment. In contrast, if the correlation is a negative correlation, the presence of said correlation indicates that patients yielding samples having an increased amount of the protein or polypeptide of interest, or the ligand therefor, or of the nucleic acid of interest are less likely to respond to said treatment. Additionally, molecular attributes of nucleic acids and/or polypeptides of the invention may correlate positively or negatively with patients' responses to therapeutic compositions and treatments, and methods to screen for the relevant molecular attributes to stratify patients to determine optimal therapeutic courses are also part of the invention.

The response(s) that are measured in these methods can be desirable response(s), in which case it is preferred to provide the therapeutic composition and/or treatment to patients having a relatively high level of the protein or polypeptide of interest, or the ligand therefor, or of the nucleic acid of interest present. Alternatively, the response(s) that are measured in these methods can be undesirable response(s), in which case it is preferred to avoid providing the therapeutic composition and/or treatment to patients having a relatively high level of the protein or polypeptide of interest, or the ligand therefor, or of the nucleic acid of interest.

The assays for the preceding methods may be performed at a laboratory to which patient-derived samples or delivered, or at the site of patient treatment. In the latter instance, kits for performing one or more assays of the invention are preferred. Antibodies, polypeptides and proteins, ligands therefor and nucleic acid probes and primers of the invention can be provided in kit form, e.g., in a single or separate container, along with other reagents, buffers, enzymes or materials to be used in practicing at least one method of the invention. Such kits can be provided in a container that can optionally include instructions or software for performing a method of the invention. Such instructions or software can be provided in any language or human- or machine-readable format.

COMPOUND SCREENING, INCLUDING HIGH-THROUGHPUT ASSAYS

The nucleic acids, proteins, polypeptides, antibodies and transgenic animals of the invention may be used to validate the role of a gene product of interest in a particular disease, disorder or undesirable response, and to screen for conditions or compounds that can be used to treat such diseases, disorders and undesirable responses, preferably using high-throughput screening methods such as they are known in the art or later developed. Such treatment can be remedial, therapeutic, palliative, rehabilitative, preventative, impeditive or prophylactic in nature. Diseases and disorders to which the invention may be applied include diabetes, including type I and type II.

The term “undesirable response” refers to a biological or biochemical response by one or more cells of an organism to one or more physical conditions, chemical agents, or combinations thereof that leads to an undesirable consequence. An undesirable response can occur at the organellar level (e.g., loss of Δ_{ψ} in mitochondria), the cellular level (e.g., cell death such as apoptosis or necrosis), in tissues (e.g., ischemia), in organs (e.g., ischemic heart disease) or to the organism as a whole (e.g., death; loss of reproductive capacity or cognitive processes).

Physical conditions that may produce an undesirable response include, without limitation, hypothermia, hyperthermia, dehydration, exposure to ultraviolet and other types of radiation, micro-gravity, physical trauma, tensile stress, and exposure to electrical or magnetic fields. Chemical agents that may produce an undesirable response include without limitation reactive oxygen species (ROS), apoptogens, and the like.

Nucleic acids of the invention are used to screen for conditions or compounds that can be used to treat disease states and undesirable responses in the following manner. Treatment of cells with antisense molecules, including ribozymes, or introduction thereinto of antisense constructs specific for a given gene product of interest, should result in such cells demonstrating at least one of the biochemical or biological defects associated with the disease or disorder for which the gene product is being validated. In like fashion, transgenic animals comprising constructs directing the over-expression of a gene of interest, or an antisense or ribozyme expression construct, or animals to which antisense, ribozyme or molecular decoy oligonucleotides are administered, will demonstrate at least one of the biochemical or biological defects associated with the disease or disorder of interest if the nucleic acid encodes a gene product that is a valid target for the disease or disorder. In addition, SNPs or mutant forms of the gene identified by the invention and correlated with diseases or disorders may be introduced into cells or animals by homologous recombination. Such cells or animals or cells derived from such animals, may be used to assess responses to conditions or compounds that can be used to treat disease states by any of a variety of assays or physiological assessments/measurements.

Similarly, for polypeptides of interest that may be targets for therapeutic intervention, cells may be contacted with one or more antibodies specific for the polypeptide, and the presentation of responses associated with the disease or disorder will be seen with valid targets. Polypeptides and proteins of the invention are also used to screen for conditions or compounds that can be used to treat disease states and undesirable responses. In one type of screen, the protein of interest, or a polypeptide derived therefrom having at least one activity of the protein of interest, is produced by recombinant DNA methods or in vitro synthetic techniques. The protein or polypepeptide, which may be attached to a solid support, is contacted with a detectably labeled ligand (including, for example, an antibody). A compound is then introduced to the reaction vessel, and active compounds are identified as those that cause the release of the detectably labeled ligand.

Assays involving nucleic acids, polypeptides, or antibodies of the invention may be automated for rapid screening of multiple compounds. The invention includes high throughput screens that may be developed as having particular applicability to the nucleic acids, polypeptides, antibodies, and genetically manipulated cells of the invention, and also high throughput screens as they are currently known in the art (for example, Stockwell, B R et al. (1999) Chem. Biol. 6: 71-83; McDonald, O B et al. (1999) Anal. Biochem. 268: 318-329; Sapolsky, R J et al. Genet. Anal. (1999) 14: 187-192; Swartzmann, E E et al. (1999) Anal. Biochem. 271: 143-151; Gonzalez, J E and Neglescu P A (1998) Curr. Opin. Biotech. 624-631), and as may be adapted for the purposes of the invention.

THERAPEUTIC APPLICATIONS

Therapeutic agents derived therefrom according to the above embodiments can be employed in combination with conventional excipients, i.e., pharmaceutically acceptable organic or inorganic carrier substances suitable for parenteral application which do not deleteriously react with the active compound. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethylcellulose, polyvinylpyrrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol, and/or dextran. Optionally, the suspension may also contain stabilizers (see generally WO 98/13353 to Whitney, published Apr. 2, 1998).

The term “therapeutically effective amount,” for the purposes of the invention, refers to the amount of a therapeutic agent which is effective to achieve its intended purpose. While individual needs vary, determination of optimal ranges for effective amounts of a therapeutic agent is within the skill of the art. Human doses can be extrapolated from animal studies (Fingle and Woodbury, Chapter 1 in Goodman and Gilman's The Pharmacological Basis of Therapeutics, 5th Ed., MacMillan Publishing Co., New York (1975), pages 1-46). Generally, the dosage required to provide an effective amount of the composition, and which can be adjusted by one of ordinary skill in the art will vary, depending on the age, health physical condition, weight, extent of disease of the recipient, frequency of treatment and the nature and scope of the desired effect.

Therapeutic agents of the invention can be delivered to mammals via intermittent or continuous intravenous injection of one or more these compositions or of a liposome (Rahman and Schein, in Liposomes as Drug Carriers, Gregoriadis, ed., John Wiley, New York (1988), pages 381-400; Gabizon, A., in Drug Carrier Systems, Vol. 9, Roerdink et al., eds., John Wiley, New York, 1989, pp. 185-212) microparticle (Tice et al., U.S. Pat. No. 4,542,025), or a formulation comprising one or more of these compositions; via subdermal implantation of drug-polymer conjugates (Duncan, Anti-Cancer Drugs 3:175-210, 1992; via microparticle bombardment (Sanford et al., U.S. Pat. No. 4,945,050); via infusion pumps (Blackshear and Rohde, in: Drug Carrier Systems, Vol. 9, Roerdink et al., eds., John Wiley, New York, 1989, pp. 293-310) or by other appropriate methods known in the art (see, generally, Remington's Pharmaceutical Sciences, 18th Ed., Gennaro, ed., Mack Publishing Co., Easton, Pa., 1990).

TRANSGENIC ANIMALS

Transgenic animals, modified with regards to a nucleic acid of interest, may be prepared. Such animals are useful for developing animal models of human disease and for evaluating the safety and effectiveness of therapeutic agents of the invention. In general, such transgenic animals are of four types: (i) “transgenic knock-outs,” in which the animal's homologs of a gene of interest are disrupted or removed, with a resulting loss of function of the corresponding gene product; (ii) “constitutive transgenics,” in which the gene of interest in operably linked to a constitutive promoter, (iii) “regulatable transgenics,” in which the gene of interest is operably linked to an inducible promoter; and (iv) “replacement transgenics,” in which the animal's homolog of the gene of interest has been replaced with the human gene of interest, or with an alternate form, for example a mutated form, of the gene of interest, which may be expressed from an endogenous or inducible promoter.

The non-human transgenic animals of the invention comprise any animal that can be genetically manipulated to produce one or more of the above-described classes of transgenic animals. Such non-human animals include vertebrates such as rodents, non-human primates, sheep, dog, cow, amphibians, reptiles, etc. Preferred non-human animals are selected from non-human mammalian species of animals, including without limitation animals from the rodent family including but not limited to rats and mice, most preferably mice (see, e.g., U.S. Pat. Nos. 5,675,060 and 5,850,001). Other non-human transgenic animals that may be prepared include without limitation rabbits (U.S. Pat. No. 5,792,902), pigs (U.S. Pat. No. 5,573,933), bovine species (U.S. Pat. Nos. 5,633,076 and 5,741,957) and ovine species such as goats and sheep (U.S. Pat. Nos. 5,827690; 5,831,141; and 5,849,992).

In one aspect of the present invention, animals, such as mice or rats, that have identified PGC and/or NRF genes can be engineered such that the animal PGC and/or animal NRF is “knocked out” and replaced with the human version. Such mice can be made using homologous recombination. These animals can be compared to their non-engineered counterparts to evaluate the activity of the human PGC and/or human NRF.

The transgenic animals of the invention are animals into which has been introduced by nonnatural means (ie., by human manipulation), one or more genes that do not occur naturally in the animal, e.g., foreign genes, genetically engineered endogenous genes, etc. The nonnaturally introduced genes, known as transgenes, may be from the same species as the animal but not naturally found in the animal in the configuration and/or at the chromosomal locus conferred by the transgene, or they may be from a different species. Transgenes may comprise foreign DNA sequences, i.e., sequences not normally found in the genome of the host animal. Alternatively or additionally, transgenes may comprise endogenous DNA sequences that are abnormal in that they have been rearranged or mutated in vitro in order to alter the normal in vivo pattern of expression of the gene, or to alter or eliminate the biological activity of an endogenous gene product encoded by the gene. (Watson et al., in Recombinant DNA, 2d Ed., W. H. Freeman & Co., New York, 1992), pages 255-272; Gordon, Intl. Rev. CytoL 115:171-229, 1989; Jaenisch, Science 240:1468-1474, 1989; Rossant, Neuron 2:323-334, 1990). Transgenes may be introduced into the genome by homologous recombination, whereby the transgene replaces the endogenous copy of the gene in the recipient animal's genome. Methods of generating and screening targeted gene replacements and the generation of transgenic animals carrying targeted gene replacements are described in U.S. Pat. No. 5,814,300.

The transgenic non-human animals of the invention are produced by introducing transgenic constructs comprising sequences of interest, or the host animal's homologs thereof, into the germline of the non-human animal. Embryonic target cells at various developmental stages are used to introduce the transgenes of the invention. Different methods are used depending on the stage of development of the embryonic target cell(s).

Microinjection of zygotes is the preferred method for incorporating transgenes into animal genomes in the course of practicing the invention. A zygote, a fertilized ovum that has not undergone pronuclei fusion or subsequent cell division, is the preferred target cell for microinjection of transgenic DNA sequences. The murine male pronucleus reaches a size of approximately 20 micrometers in diameter, a feature which allows for the reproducible injection of 1-2 picoliters of a solution containing transgenic DNA sequences. The use of a zygote for introduction of transgenes has the advantage that, in most cases, the injected transgenic DNA sequences will be incorporated into the host animal's genome before the first cell division (Brinster et al., Proc. Natl. Acad. Sci. USA. 82:4438-4442, 1985). As a consequence, all cells of the resultant transgenic animals (founder animals) stably carry an incorporated transgene at a particular genetic locus, referred to as a transgenic allele. The transgenic allele demonstrates Mendelian inheritance: half of the offspring resulting from the cross of a transgenic animal with a non-transgenic animal will inherit the transgenic allele, in accordance with Mendel's rules of random assortment.

Viral integration can also be used to introduce the transgenes of the invention into an animal. The developing embryos are cultured in vitro to the developmental stage known as a blastocyte. At this time, the blastomeres may be infected with appropriate retroviruses (Jaenisch, Proc. Natl. Sci. U.S.A. 73:1260-1264, 1976; Soriano and Jaenisch, Cell 46:19-29, 1986). Infection of the blastomeres is enhanced by enzymatic removal of the zona pellucida (Hogan, et al., in Manipulating the Mouse Embryo, Cold Spring Harbor Press, Cold Spring Harbor, N.Y., 1986). Transgenes are introduced via viral vectors which are typically replication-defective but which remain competent for integration of viral-associated DNA sequences, including transgenic DNA sequences linked to such viral sequences, into the host animal's genome (Jahner et al., Proc. Natl. Acad. Sci. U.S.A. 82:6927-6931, 1985; Van der Putten et al., Proc. Natl. Acad. Sci. U.S.A. 82:6148-6152, 1985). Transfection is easily and efficiently obtained by culture of blastomeres on a mono-layer of cells producing the transgene-containing viral vector (Van der Putten et al., Proc. Natl. Acad. Sci. U.S.A. 82:6148-6152, 1985; Stewart, et al., EMBO J. 6:383-388, 1987). Alternatively, infection may be performed at a later stage, such as a blastocoele (Jahneret al., Nature 298:623-628, 1982). In any event, most transgenic founder animals produced by viral integration will be mosaics for the transgenic allele; that is, the transgene is incorporated into only a subset of all the cells that form the transgenic founder animal. Moreover, multiple viral integration events may occur in a single founder animal, generating multiple transgenic alleles which will segregate in future generations of offspring. Introduction of transgenes into germline cells by this method is possible but probably occurs at a low frequency (Jahner et al., Nature 298:623-628, 1982). However, once a transgene has been introduced into germline cells by this method, offspring may be produced in which the transgenic allele is present in all of the animal's cells, i.e., in both somatic and germline cells.

Embryonic stem (ES) cells can also serve as target cells for introduction of the transgenes of the invention into animals. ES cells are obtained from pre-implantation embryos that are cultured in vitro (Evans et al., Nature 292:154-156, 1981; Bradley et al., Nature 309:255-258, 1984; Gossler et al., Proc. Natl. Acad. Sci. U.S.A. 83:9065-9069, 1986; Robertson et al., Nature 322:445-448, 1986; Robertson, E.J., in Teratocarcinomas and Embryonic Stem Cells: A Practical Approach, Robertson, E. J., ed., IRL Press, Oxford, 1987, pp. 71-112). ES cells, which are commercially available (from, e.g., Genome Systems, Inc., St. Louis, Mo.), can be transformed with one or more transgenes by established methods (Lovell-Badge, R. H., in Teratocarcinomas and Embryonic Stem Cells: A Practical Approach, Robertson, E. J., ed., IRL Press, Oxford, 1987, pp. 153-182). Transformed ES cells can be combined with an animal blastocyst, whereafter the ES cells colonize the embryo and contribute to the germline of the resulting animal, which is a chimera (composed of cells derived from two or more animals) (Jaenisch, Science 240:1468-1474, 1988; Bradley in: Teratocarcinomas and Embryonic Stem Cells: A Practical Approach, Robertson, E. J., ed., IRL Press, Oxford 1987, pp. 113-151). Again, once a transgene has been introduced into germline cells by this method, offspring may be produced in which the transgenic allele is present in all of the animal's cells, i.e., in both somatic and germline cells.

However it occurs, the initial introduction of a transgene is a non-Mendelian event. However, the transgenes of the invention may be stably integrated into germ line cells and transmitted to offspring of the transgenic animal as Mendelian loci. In mosaic transgenic animals, some cells carry the transgenes and other cells do not. In mosaic transgenic animals in which germ line cells do not carry the transgenes, transmission of the transgenes to offspring does not occur. Nevertheless, mosaic transgenic animals are capable of demonstrating phenotypes associated with the transgenes.

Offspring that have inherited the transgenes of the invention are distinguished from littermates that have not inherited transgenes by analysis of genetic material from the offspring for the presence of biomolecules that comprise unique sequences corresponding to sequences of, or encoded by, the transgenes of the invention. For example, biological fluids that contain polypeptides uniquely encoded by the transgenes of the invention may be immunoassayed for the presence of the polypeptides. A more simple and reliable means of identifying transgenic offspring comprises obtaining a tissue sample from an extremity of an animal, e.g., a tail, and analyzing the sample for the presence of nucleic acid sequences corresponding to the DNA sequence of a unique portion or portions of the transgenes of the invention. The presence of such nucleic acid sequences may be determined by, e.g., hybridization (“Southern”) analysis with DNA sequences corresponding to unique portions of the transgene, analysis of the products of PCR reactions using DNA sequences in a sample as substrates and oligonucleotides derived from the transgene's DNA sequence, etc.

Cloned animals, transgenic and otherwise, of the invention may also be prepared (for a review of mammalian cloning techniques, see Wolf et al., J. Assist. Reprod. Genet. 15:235-239, 1998). Such cloned animals include, without limitation, ovine species such as sheep (Campbell et al., Nature 380:64-66, 1996; Wells et al., Biol. Reprod. 57:385-393, 1997) rodents such as mice (Wakayama et al., Nature 394:369-374, 1998) and non-human primates such as rhesus monkeys (Meng et al., Biol. Reprod. 57:454-459, 1997).

The transgenic and cloned animals of the invention may be used as animal models of human disease states and to evaluate potential therapies for such disease states. For example, in such methods, a first transgenic animal having a disease state (or one or more symptomatic components thereof) is given a known dose of a candidate therapeutic composition or exposed to a candidate therapeutic treatment, and a second (control) transgenic animal is given a placebo or not exposed to the candidate therapeutic treatment. Symptoms and/or clinical end-points relevant to the disease state are measured in both animals over appropriate intervals of time, and the results are compared. Therapeutic (desirable) compositions and treatments are identified as those which ameriolate, delay the onset of or eliminate such symptoms and end-points in the treated animal relative to the control animal. In like fashion, undesirable compositions and treatments that aggravate or accelerate the disease state are identified as those which enhance the degree of such symptoms and end-points and/or hasten their onset. Because of their high degree of genetic identity, cloned transgenic animals are preferred in such methods.

### EMBODIMENTS OF THE INVENTION

I. METHODS FOR INCREASING MITOCHONDRIAL MASS OR AT LEAST ONE MITOCHONDRIAL FUNCTION IN A CELL.

In certain embodiments the present invention provides a method to increase mitochondrial mass or increase at least one mitochondrial function in cells, particularly ex vivo or in vivo. The present invention is not limited to any particular cell type, disease or disorder. Preferably, the present invention increases mitochondrial mass or increases at least one mitochondrial function in diabetic or prediabetic cells or subjects (diabetes type I or diabetes type II), particularly in insulin producing cells or glucose responsive cells. Such increase in mitochondrial mass or at least one mitochondrial function can preferably be accomplished by regulating the transcription, translation or activity of NRF or PGC.

Thus, the invention provides a method for treating diabetes that includes increasing mitochondrial mass or improving at least one mitochondrial function in cells in a subject in need thereof. This method can be accomplished in any number of ways, including providing appropriate stimuli, compounds or compositions, including small molecules, polypeptides, nucleic acid molecules, gene therapy constructs or organic molecules, compounds or compositions identified using a method of the present invention or combinations thereof.

Increasing mitochondrial mass or improving at least one mitochondrial function in a cell can be accomplished in any manner. Preferably, the mitochondrial mass being increased is of metabolically fumctional (e.g., aerobic respiration-competent) mitochondria, and not of respiration uncoupled (e.g., wherein oxidative phosphorylation is uncoupled from ATP synthesis) mitochondria, such that ATP production within the cell is increased. However, mitochondria can be uncoupled to some degree, for example, by uncoupling factors such as UCP's (Wu et al., Cell 98:115-124 (1999)). Alternatively, mitochondrial function in increased such that ATP production within the cell is increased. Not wanting to be limited to theory, the increase in ATP production related to the increase in mitochondrial mass or function in insulin producing cells results in an increase in insulin production and/or insulin secretion. Alternatively, the increase in ATP production can increase the sensitivity of insulin sensitive cells to insulin.

The cells can be any cells within the subject, preferably insulin producing cells or insulin sensitive cells. Preferred insulin producing cells are pancreatic cells, such as within the islets of Langerhans, preferably the beta cells. Preferred insulin sensitive cells are those cells involved in glucose metabolism, homeostasis and/or storage, such as liver cells and/or muscle cells. One additional benefit to increasing mitochondrial mass or function in liver cells is that the activity of the liver can increase such that these cells can perform detoxification functions, such as for reducing the toxicity or increasing the solubility of compounds, including therapeutics such as antiviral compounds and antisense compounds. In addition, subjects that have liver diseases or disorders, such as hepatitis, cirrhosis, toxic intake of compounds, can have their liver function increased using the methods of the present invention.

In certain embodiments of the present invention, the subject and/or the cells are treated with at least one agent that enhances at least one activity of a NRF or PGC gene or polypeptide. Agents that increase the activity of a NRF gene or PGC gene are those that can directly or indirectly increase the transcription of such gene, modulate post-transcriptional modification or mRNA half-life. Examples of such compounds can include cold and caloric intake. Alternatively, the cell or subject can include a nucleic acid molecule that can be induced to increase the transcription of endogenous or exogenous NRF or PGC genes. For example, such constructs can include a NRF gene or PGC gene operably linked to an inducible or constitutive promoter such that NRF or PGC transcription can be increase in a regulated or non-regulated fashion.

NRF can be any NRF, such as rat, mouse or human, such as NRF-1 and NRF-2. A NRF can have at least one activity of a NRF, preferably the coactivation of a PGC such as PGC-1 which can modulate the trancription of mtTFA (mitochondrial transcription factor A) that can lead to mitochondrial biogenesis and enhanced transcription of the mitochondrial genome. Preferably, such mitochondrial biogenesis and enhanced transcription of the mitochondrial genome results in the enhanced production of ATP. Various NRF nucleic acid sequences and amino acid sequences from a variety of biological sources are provided in SEQ ID NO:1, SEQ ID NO:2, SEQ ID NO:3, SEQ ID NO:4, SEQ ID NO:7, SEQ ID NO:8, SEQ ID NO:9 and SEQ ID NO:10. These sequences or portions thereof or related sequences as described herein that include at least one activity of a NRF can be used in the present invention.

The activity of NRF-1 is regulated by phosphorylation at serine residues 39, 44, 46, 47, and 52 (Gugneja and Scarpulla, J Biol Chem 272:18732-18739, 1997). Although the kinase(s) responsible for the phosphorylation in vivo is not known, casein kinase II produces an identical phosphorylation pattern in vitro to that observed in vivo. PGC-1 may also be regulated by phosphorylation, since its sequence contains three consensus phosphorylation sites for protein kinase A. Hence, one method to modulate mitochondrial biogenesis may be through exploiting the relevant phosphorylation cascade. To identify members of the phosphorylation signaling pathway (e.g., Gogneja et al., 1997 J Biol Chem 272:18732-18739) such as signaling molecules that may be used to alter, modulate or otherwise regulate mitochondrial biogenesis, a cell model is selected in which mitochondrial biogenesis can be stimulated. As examples, cultured skeletal muscle cells may be used with electrical stimulation or thyroid hormone as the stimulus for mitochondrial biogenesis. Alternatively, a fat cell culture such as 3T3-L1 cells may be used, with norepinephrine providing the stimulus for mitochondrial biogenesis. Alternatively, cultured cells such as HeLa or HEK293 that express PGC-l and/or NRF-1 under a tetracycline inducible system may be used, wherein induced expression of PGC-1 and/or NRF-1 stimulates mitochondrial biogenesis. After sufficient time with the appropriate stimulus to allow induction (1-2 days), the cells are incubated with [³²P]orthophosphate for 4 hrs. Cells are then harvested and subjected to SDS-PAGE to resolve the labeled proteins. Alternatively, known members of phosphorylation cascades (including MAP, MAPK, jun, etc.) can be immunoprecipitated from the cell lysates using appropriate antibodies against signaling molecules, phosphoserine, or phosphotyrosine. The induced and non-induced (stimulated) cells are then compared. Those proteins whose phosphorylation is increased (or decreased) in the induced versus the non-induced cells are candidate signaling molecules. Proteins that cannot be identified by immunoprecipitation with antibodies to known proteins may be cut from the gels and partially sequenced to reveal their identities. Novel proteins will likely require complete sequencing. The proposed role of the candidate signaling proteins can be validated by traditional overexpression or knockout approaches to ascertain the effects of such manipulations on mitochondrial biogenesis in the engineered cell lines. This approach ultimately identifies additional molecules whose expression or activity can be modulated to enhance mitochondrial biogenesis.

An alternative approach is to identify the relevant signaling molecules, including protein kinases, phosphatases, co-factors, activators, inducers and the like, that regulate PGC-1 and/or NRF-1 phosphorylation. The presence of protein kinase A (PKA) consensus phosphorylation sites in PGC-1 implicates one of the PKAs or a related kinase. One method to identify the relevant protein kinase that phosphorylates PGC-1 (or NRF-1) is described briefly: Knockout or transgenic mice that lack various forms of PKA, or that express mutant PKA, are available or can be produced according to well known methodologies. Without wishing to be bound by theory, if PGC-1 phosphorylation is a regulator of mitochondrial biogenesis, then its phosphorylation would be expected to increase in brown fat of mice exposed to cold, or in skeletal muscle of mice following exercise (e.g., electrochemical stimulation at neuromuscular junctions), or in appropriate receptor-bearing cells following adrenergic stimulation (e.g., Boss et al., 1999 Biochem. Biphys. Res. Comm. 261:870). Hence, control and transgenic mice may be exposed to one of these stimuli, the appropriate tissues harvested, and the degree of phosphorylation of PGC-1 interrogated using antibodies specific for PGC-1 and phosphoserine. Specifically, tissue may be prepared in the presence of kinase and phosphatase inhibitors to preserve the in vivo phosphorylation state of PGC-1, which may be determined according to any of a variety of well known procedures, for example, by immunoprecipitation using anti-PGC-1 antibody followed by electrolphoresis and western immunoblotting, using an anti-phosphoserine antibody. If the transgenic mice with deficient or absent PKA demonstrate less phosphorylation of PGC-1 than do normal mice, then one could conclude that PKA has a role in controlling mitochondrial biogenesis. Agents that regulate PKA are then screened for their ability to enhance mitochondrial biogenesis. In an alternate approach, recombinant PGC-1 immobilized on a solid support (through, for example, binding of a polyHis tag to Ni-agarose) may be in vitro phosphorylated by tissue lysates from normal and PKA-deficient animals to determine whether the degree of PGC-1 phosphorylation differs. Similar studies could be employed to interrogate the role of other protein kinases in PGC-1 and NRF-1 phosphorylation.

PGC can be any PGC, such as rat, mouse or human, such as PGC-1. A PGC can have at least one activity of a PCG, preferably the coactivitation of a NRF such as NRF- 1 which can modulate the trancription of mtTFA (mitochondrial transcription factor A) that can lead to mitochondrial biogenesis and enhanced transcription of the mitochondrial genome. Preferably, such mitochondrial biogenesis and enhanced transcription of the mitochondrial genome results in the enhanced production of ATP. A variety of PGC nucleic acid sequences and amino acid sequences from a variety of sources are provided in SEQ ID NO:5, SEQ ID NO:6, SEQ ID NO:11 and SEQ ID NO:12. These sequences, or portions thereof, or related sequences as described herein that include at least one activity of a PGC, can be used in the present invention.

II. METHODS FOR SCREENING FOR TEST COMPOUNDS THAT INCREASE MITOCHONDRIAL MASS OR THAT INCREASE MITOCHONDRIAL FUNCTION.

As provided herein, according to certain embodiments the present invention provides a method to screen for compounds that increase mitochondrial mass or increase mitochondrial fuinction, particularly ex vivo or in vivo. The present invention is not limited to a particular mechanism cell type, disease state or disorder. Preferably, mitochondrial mass or mitochondrial function is increased in cells that are prediabetic or diabetic in nature, particularly insulin producing cells, including glucose responsive cells (diabetes type I or diabetes type II). Such increase in mitochondrial mass or function can be accomplished by regulating the transcription, translation or activity of NRF or PGC.

One embodiment of the present invention is a method for screening for identifying test compounds that influence the expression of a nucleic acid that encodes a NRF protein or a PGC protein, that includes contacting at least one cell that includes a nucleic acid molecule that encodes a NRF protein or a PGC protein with one or more test compounds; and measuring the expression of an NRF protein or a PGC protein.

The cell used in the methods of the present invention can be any cell including, preferably, a cell that is insulin producing or insulin sensitive, and preferably cells in culture, such as continuous cell lines. In addition, cells from whole organisms, including cells in suspension or from a tissue or organ or fluid from an organism, such as Zucker diabetic fatty rats (ZDF's), preferably pancreatic cells such as beta cells can be used. For insulin producing cells, the rat cell line INS 1 is preferred. Other preferred cells include SY5Y cells, HEK293 cells, G7/V79 cells, rho⁰ 3T3-L1, rho⁰ INS-1 and NRF-1/293 cells. For insulin sensitive cells, muscle cells or liver cells are preferred as they are known in the art, such as HEPG2 cells.

The nucleic acid molecules that encode a PGC or NRF can be endogenous to the genome of the cell or can be engineered into the genome such as by homologous recombination or by random integration (Whitney et al., W098/13353, published Apr. 2, 1998, Smith et al., WO 94/24301, published Oct. 27, 1994). When endogenous, the expression of the NRF or PGC can be enhanced using stimuli or compounds known or expected to enhance such expression. When randomly integrated, such nucleic acid molecules can be operably linked to an endogenous regulatory element or an exogenous regulatory element that can be modulated in the presence of an inducer or repressor, such as 2XTetO₂. Optionally, the NRF gene or PGC gene can be operably linked to a detectable reporter gene, such as green fluorescent protein, beta-lactamase or luciferase, for example, or to a detectable tag, for example, an affinity tag defined by a specific binding partner or an epitope tag defined by a cognate antibody such as FLAG, such that the expression the NRF gene or PGC gene can be monitored by measuring the expression of the reporter gene or tag.

In the case of exogenous NRF or PGC genes, the genes can be operably linked to a regulatory element to form a regulatory expression construct that is extrachromosomal, such as a plasmid. The expression of the NRF or PGC gene in the regulatory expression construct can be modulated by a repressor or inducer of the regulatory element. Optionally, and in certain preferred embodiments, the NRF gene or PGC gene in the regulatory expression construct can be operably linked to a reporter gene, such as green fluorescent protein, beta-lactamase or luciferase, for example, or a tag, such as FLAG, such that the expression the NRF gene or PGC gene can be monitored by measuring the expression of the reporter gene or tag in vitro, ex vivo or in vivo. The NRF gene and PGC gene, when provided together in the same cell, can be on the same or on different extrachromosomal elements. Such general technology is known in the art (e.g., U.S. Pat. No. 5,298,429 to Evans issued Mar. 29, 1994).

As discussed above, the expression of NRF and/or PGC can be measured using a variety of methods (in vitro, ex vivo or in vivo), including reporter genes or tags, such as immunological tags. In addition, other detection methods, such as Northern blots or Southern blots can be used. Furthermore, nucleic acid amplification methods, such as PCR, such as quantitative PCR or RT-PCR can be used. Also, in situ hybridization methods or immunohistochemical or other receptor-ligand reactions can be used.

Alternatively, the activity of a NRF or PGC can be directly measured, such as PGC binding to its regulatory element or NRF binding to regulatory elements of cytochrome c or COX, or other methods as they are known in the art. Compounds that modulate NRF or PGC activity can also presumptively modulate mitochondrial biogenesis, ATP synthesis, insulin production or insulin secretion, among other

The cells of the present invention can be contacted with one or more test chemicals. The expression of NRF or PGC or both in the cells can be monitored and test compounds that increase such expression can be identified. Alternatively, test compounds that increase at least one mitochondrial activity, induce mitochondrial biogenesis, increase the production of ATP, increase the synthesis or secretion of insulin or increase the insulin sensitivity of the cell can be monitored using methods known in the art. Test compounds having such activity can be identified and screened for other activities described herein. Preferably, at least one measure of mitochondrial activity as discussed herein is measured, more preferably between about two and about five measures of mitochondrial activity. Preferably, the measures of mitochondrial activity are selected from those described herein, such as cytochrome c oxidase activity, ATP levels, malate dehydroginase activity, rate of ATP synthesis or mitochondrial number, but are not limited thereto

General Materials and Methods

1. Expression Constructs and Cells

Nucleic acid molecules of the present invention can be provided as part of an expression construct. An expression construct is a nucleic acid molecule that includes expression control sequences, such as promoters, appropriate for the expression of a nucleic acid molecule in an appropriate expression system. Preferably, a nucleic acid molecule of the present invention is operably linked to an expression control sequence, such as a promoter, that is appropriate for a particular expression system, such as an in vitro expression system or a host cell, such as a bacterial or eukaryotic cell.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal binding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

A nucleic acid molecule can be engineered into an expression construct, such as a plasmid or viral vector, using methods known in the art (Sambrook et al., supra, 1989). The nucleic acid molecule is preferably inserted in-frame and in the proper orientation in the expression construct such that a polypeptide of appropriate amino acid sequence relative to the native polypeptide coded by the nucleic acid is produced upon expression thereof. Such in-frame insertions can be inferred from the nucleotide sequence of a nucleic acid molecule and be confirmed using a variety of methods, including computer anlaysis of predicted amino acid sequences and the folding thereof, or by binding with antibodies that specifically bind with identified or orphan proteins, such as unidentified proteins or portions of proteins that do not have an identified function.

The nucleic acid molecules of the invention, preferably in an expression construct, can be inserted into a host cell, such as a prokaryotic cell (such as a bacterium such as E. coli) or a eukaryotic cell (such as a HeLa cell) using methods known in the art, such as electroporation or treatment with cold calcium solutions. The expression construct is preferably configured such that an expression control element, such as a promoter, is operably linked to a nucleic acid molecule of the present invention in-frame and in the proper orientation such that the native amino acid sequence encoded by the nucleic acid molecule of the present invention are expressed by the expression construct. Expression constructs can be chosen such that the nucleic acid molecule of the present invention is expressed efficiently in a chosen host cell. The products of the expressed nucleic acid of the present invention, including RNA transcripts and at least one polypeptide, can be collected and identified using methods known in the art. “RNA transcripts” are RNA molecules that are synthesized (“transcribed”) by RNA polymerase using DNA as a template.

2. Gene Therapy Constructs

Another aspect of the present invention is a gene therapy construct that includes an expression vector that includes a promoter operably linked to at least one nucleic acid of the present invention. Preferably, the nucleic acid of the present invention is selected from a) a substantially pure nucleic acid molecule including at least one of SEQ ID NO:1 through SEQ ID NO:12 and reverse complements thereof, a cDNA molecule prepared by a method of the present invention and reverse complements thereof.

The gene therapy construct is preferably a viral vector, such as a retrovirus, adenovirus, adenoassociated virus, papilloma virus or other type of virus vector used in gene therapy systems or genetic manipulation of cells. Preferred gene therapy constructs include those that can target insulin producing cells or insulin sensitive cells. Coxsakievisus, particular Coxsackievirus B and Coxsackievirus B4, Echoviruses, such as Echo 11, certain adenoviral vectors and certain retroviruses, such as C-type retroviruses, can target pancreatic cells, such as beta cells (Ramsingh et al., Bioessays 19:793-800 (1997), Hyoty et al., Clin. Diagn. Virol. 9:77-84 (1998), Jenson et al., Lancet 2(8190):354-358 (1980), Luppi et al., J. Biol. Regul. Homeost. Agents 13:14-24 (1999), Tsumura et al., Lab. Anim. 32:86-94 (1998), Frisk et al., Virus Res. 33:229-240 (1994), Giannoukakis et al. Diabetes 48:1730-1736 (1999)). In addition, liposomes and lipid preparations can also be used as vectors. A variety of these types of vectors are known in the art (see, for example: U.S. Pat. No. 5,399,346 to Anderson et al., issued Mar. 21, 1995; Bandara et al., DNA and Cell Biology, 11:227-231 (1992); Berkner, Biotechniques 6:616-629 (1989); U.S. Pat. No. 5,240,846 to Collins et al., issued Aug. 31, 1993; Culver and Blaese, TIG 5:171-178 (1994); Goldman et al., Gene Therapy 3:811-818 (1996); Hamada et al., Gynecologic Oncology 63:219-227 (1996); Holmberg et al., J. Liposome Res. 1:393-406 (1990); Hurford et al., Nature Genetics 10:430-435 (1995); Karlsson et al., EMBO J. 5:2377-2385 (1986); Kleinerman et al., Cancer Res. 55:2831-2836 (1995); Krul et al., Cancer Immunol. Immunother. 43:44-48 (1996); U.S. Pat. No. 5,532,220 to Lee et al., issued Jul. 2, 1996; Liu et al., Nature Biotechnology 15:167-173 (1997); Mathiowitz et al., Nature 386:410-(1997); Nabel et al. Proc. Natl. Acad. Sci. USA 90:11307-11311 (1993); Nabel et al., Science, 14 Sep:1285-1288 (1990); Ram et al., Cancer Res. 53:83-88 (1993); Rosenfeld et al., Cell 68:143-155 (1992); U.S. Pat. No. 5,580,859 to Felgner et al., issued Dec. 30, 1997; WO 98/13353 to Whitney et al., published Apr. 2, 1998; U.S. Pat. No. 5,298,429 to Evans et al., issued Mar. 29, 1994; U.S. Pat. No. 5,514,561 to Quante et al., issued May 7, 1996; WO 96/24301 to The University of Edinburgh, published Oct. 27, 1994; WO 96/30540 to The Regents of the University of California, published Oct. 3, 1996; Larrick and Burck, Gene Therapy, Application of Molecular Biology, Elsevier, N.Y. (1991); and Pinkert, Transgenic Animal Technology, a Laboratory Handbook, Academic Press, Inc., San Diego (1994)).

Appropriate viral vectors can be selected based on the route of administration and the target cell type or population. For example, retroviruses are preferred if the target cell type or population is actively proliferating and other viruses, such as lentivirus, adeno associated virus, adenoviruses, are preferred if the target cell type or population is not actively proliferating (see, for example, Larrick et al, Gene Therapy, Elsevier, N.Y. (1991)). Different viruses have different specificity for different cell types and populations. Thus, viruses that infect a targeted cell type of population of cells can be selected. The viral vector can be provided as a pharmaceutical composition in an appropriate pharmaceutically acceptable carrier, such as an exciptient, at an appropriate dose for an appropriate route of administration and regime.

The gene therapy construct can also be a naked DNA construct such as plasmids that are useful in a gene therapy treatment system (see, for example, U.S. Pat. No. 5,580,859 to Felgner et al., issued Dec. 3, 1996; U.S. Pat. No. 5,703,055 to Feigner et al., issued Dec. 30, 1997; U.S. Pat. No. 5,846,946 to Huebner et al., issued Dec. 8, 1998; and U.S. Pat. No. 5,910,488 to Nabel et al., issued Jun. 8, 1999). A particular vector can be made with a particular target tissue, cell type or population of cells in mind. For example, particular regulatory elements, such as control elements and promoters, can be chosen based on the target cells such that the regulatory elements are operable in the target cells. The vector is preferably introduced into a subject via direct injection into the pathological location, such as the brain, but other methods of delivery, such as systemic or intra-tissue or organ administration distal from the pathological location, such as the muscle, may also be used. These types of vectors can be provided as a pharmaceutical composition in an appropriate pharmaceutically acceptable carrier, such as an exipient, at an appropriate dose for an appropriate route of administration and regime.

3. Screening Methods

The present invention also includes a variety of methods to identify biologically active agents that can modulate the activity of at least one function of a polypeptide of the present invention. The functions can be in vitro (outside of a whole cell), ex vivo (within or on a cell but not in a whole organism such as samples from a whole organism or cells in culture) or in vivo (within a whole organism). The present invention also includes biologically active agents identified by these methods. Organism refers to a subject, such as a non-human animal (such as a test animal or transgenic animal) or a human.

The term “modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

The term “modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonists, partial antagonists, partial agonists, antagonists, antineoplastic agents, cytotoxic agents, inhibitors of neoplastic transformation or cell proliferation, cell proliferation promoting agents, antiviral agents, antimicrobial agents, antibacterial agents, antibiotics, and the like) by inclusion in assays described herein. The activity of a modulator may be known, unknown or partially known.

The terms “test compound” or “test chemical” refers to a chemical, compound, composition or extract to be tested by at least one method of the present invention to be a putative modulator. A test compound or test chemical identified by the present invention is a “biologically active agent.” Test compounds can include small molecules, such as drugs, proteins or peptides or active fragments thereof, such as antibodies, nucleic acid molecules such as DNA, RNA or combinations thereof, antisense molecules or ribozymes, or other organic or inorganic molecules, such as lipids, carboydrates, or any combinations thereof. Test compounds that include nucleic acid molecules can be provided in a vector, such as a viral vector, such as a retrovirus, adenovirus or adeno-associated virus, a liposome, a plasmid or with a lipofection agent. Test compounds, once identified, can be agonists, antagonists, partial agonists or inverse agonists of a target. A test compound is usually not known to bind to the target of interest. “Control test compound” refers to a compound known to bind to the target (for example, a known agonist, antagonist, partial agonist or inverse agonist). Test compound does not typically include a compound added to a mixture as a control condition that alters the function of the target to determine signal specificity in an assay. Such control compounds or conditions include chemicals that (1) non-specifically or substantially disrupt protein structure (for example chaotropes or denaturing agents such as urea or guanidinium, sulfhydryl reagents such as dithiotritol and beta-mercaptoethanol), (2) generally inhibit cell metabolism (for example mitochondrial uncouplers) or (3) non-specifically disrupt electrostatic or hydrophobic interactions of a protein (for example, high salt concentrations or detergents at concentrations sufficient to non-specifically disrupt hydrophobic or electrostatic interactions). The term test compound also does not typically include compounds known to be unsuitable for a therapeutic use for a particular indication due to toxicity to the subject. Usually, various predetermined concentrations of test compounds are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test compounds, the concentration of test chemical used can be expressed on a weight to volume basis. Under these circumstances, the following ranges of concentrations can be used: between about 0.001 micrograms/ml and about 1 milligram/ml, preferably between about 0.01 micrograms/ml and about 100 micro grams/ml, and more preferably between about 0.1 micrograms/ml and about 10 micrograms/ml.

Test compounds that modulate the activity of the at least one in vitro or ex vivo function of a polypeptide of the present invention have presumptive therapeutic activity in modulating the activity of that in vivo function in a subject, including a human. The present invention includes biologically active agents identified by a method of the present invention. Such biologically active agents can be provided as a pharmaceutical, such as with an excipient.

4. In vitro function

Another aspect of the invention involves a method for identifying biologically active agents, including: providing a sample that includes at least one polypeptide of the present invention; contacting the sample with at least one test chemical; detecting at least one in vitro function of the polypeptide; and identifying at least one test chemical that modulates (such as enhances or inhibits) the at least one in vitro function of the polypeptide. Preferably, this method is practiced in a high throughput format and device, such as described in WO 98/52047 to Stylli et al., published Nov. 19, 1998.

In operation, a polypeptide of the present invention having at least one in vitro function that is detectable using a compound that provides a readout of the at least one in vitro function, such as an enzymatic substrate that changes at least one property, such as, for example, colormetric, spectrographic or fluorescent properties, upon the action of the at least one in vitro function upon the enzymatic substrate is provided. Such enzymatic substrates are known in the art for a variety of activities, such as, for example, proteases and kinases (see, for example, WO 97/28261 to Tsien et al., published Aug. 7, 1997; WO 98/02571 to Tsien et al., published Jan. 22, 1998; and The Sigma Catalogue, Sigma Chemical Company, St. Louis, Mo. (1999)).

The polypeptide of the present invention having at least one in vitro function is contacted with a test chemical before or contemporaneously with being contacted with the compound that provides a readout for the at least one in vitro function. The at least one in vitro function is monitored by monitoring the readout of that activity. The results of these studies can be compared to an appropriate control to determine the ability of a test chemical to modulate the activity of the at least one in vitro function. Appropriate controls are known in the art, such as performing the test in the absence of the test chemical. The control can be performed at the same time as the test, but can also be performed at a time and place distant from the test. For example, standard curves or values can be obtained and provided for a particular test which can be used in the comparison.

5. Ex vivo function

Another aspect of the invention involves a method for identifying biologically active agents, including: providing a sample that includes at least one cell that includes at least one polypeptide of the present invention; contacting the sample with at least one test chemical; detecting at least one ex vivo function of the polypeptide; and identifying at least one test chemical that modulates (such as enhances or inhibits) the at least one ex vivo function of the polypeptide. The polypeptide of the present invention is preferably within or associated with a cell and the test chemical is contacted with the cell. Preferably, this method is practiced in a high throughput format and device, such as described in WO 98/52047 to Stylli et al., published Nov. 19, 1998. The at least one cell can be from a sample from a subject, such as a test animal, transgenic animal, or human, or can be a cell in culture.

In operation, a polypeptide of the present invention having at least one ex vivo function that is detectable using a compound that provides a readout of the at least one in vitro function, such as an enzymatic substrate that changes at least one property, such as, for example, colormetric, spectrographic or fluorescent properties, upon the action of the at least one ex vivo function upon the enzymatic substrate is provided. Such enzymatic substrates are known in the art for a variety of activities, such as, for example, proteases, kinases (see, for example, WO 97/28261 to Tsien et al., published Aug. 7, 1997; WO 98/02571 to Tsien et al., published Jan. 22, 1998; and The Sigma Catalogue, Sigma Chemical Company, St. Louis, Mo. (1999)).

The cell that includes at least one polypeptide of the present invention having at least one ex vivo function is contacted with a test chemical before or contemporaneously with being contacted with the compound that provides a readout for the at least one ex vivo function. The at least one ex vivo function is monitored by monitoring the readout of that activity. The results of these studies can be compared to an appropriate control to determine the ability of a test chemical to modulate the activity of the at least one ex vivo function. Appropriate controls are known in the art, such as performing the test in the absence of the test chemical. The control can be performed at the same time as the test, but can also be performed at a time and place distant from the test. For example, standard curves or values can be obtained and provided for a particular test which can be used in the comparison.

6. In vivo function

Another aspect of the invention involves a method for identifying biologically active agents, including: providing at least one subject that includes at least one polypeptide of the present invention; contacting the at least one subject with a test chemical; detecting at least one in vivo function of the polypeptide; and identifying at least one test chemical that modulates (such as enhances or inhibits) the at least one in vivo function of the polypeptide.

In operation, a polypeptide of the present invention having at least one in vivo function that is detectable using a compound that provides a readout of the at least one in vitro function, such as an enzymatic substrate that changes at least one property, such as, for example, calorimetric, spectrographic or fluorescent properties, upon the action of the at least one in vivo function upon the enzymatic substrate is provided. Such enzymatic substrates are known in the art for a variety of activities, such as, for example, proteases, kinases (see, for example, WO 97/28261 to Tsien et al., published Aug. 7, 1997; WO 98/02571 to Tsien et al., published Jan. 22, 1998; and The Sigma Catalogue, Sigma Chemical Company, St. Louis, Mo. (1999)).

The subject that includes at least one polypeptide of the present invention having at least one in vivo function is contacted with a test chemical before or contemporaneously with being contacted with the compound that provides a readout for the at least one in vivo function. The at least one in vivo function is monitored by monitoring the readout of that activity. The results of these studies can be compared to an appropriate control to determine the ability of a test chemical to modulate the activity of the at least one in vivo function. Appropriate controls are known in the art, such as performing the test in the absence of the test chemical. The control can be performed at the same time as the test, but can also be performed at a time and place distant from the test. For example, standard curves or values can be obtained and provided for a particular test which can be used in the comparison.

In the case of diabetes, a preferred animal model is the non-obese diabetic (NOD) mouse. The successful use of this animal model in diabetic drug discovery is reported in the literature (Yang et al., J. Autoimmun. 10:257-260 (1997), Akashi et al., Int. Immunol. 9:1159-1164 (1997), Suri and Katz, Immunol. Rev. 169:55-65 (1999), Pak et al., Autoimmunity 20:19-24 (1995), Toyoda and Formby, Bioessays 20:750-757 (1998), Cohen, Res. Immunol. 148:286-291 (1997), Baxter and Cooke, Diabetes Metal. Rev. 11:315-335 (1995), McDuffie, Curr. Opin. Immunol. 10:704-709 (1998), Shieh et al. Autoimmunity 15:123-135 (1993), Anderson et al., Autoimmunity 15:113-122 (1993)).

7. Pharmacology and toxicity of test compounds

The structure of a test compound can be determined or confirmed by methods known in the art, such as mass spectroscopy. For test compounds stored for extended periods of time under a variety of conditions, the structure, activity and potency thereof can be confirmed.

Identified test compounds can be evaluated for a particular activity using recognized methods and those disclosed herein. For example, if an identified test compound is found to have anticancer cell activity in vitro, then the test compound would have presumptive pharmacological properties as a chemotherapeutic to treat cancer. Such nexuses are known in the art for several disease states, and more are expected to be discovered over time. Based on such nexuses, appropriate confirmatory in vitro and in vivo models of pharmacological activity, and toxicology, can be selected and performed. The methods described herein can also be used to assess pharmacological selectivity and specificity, and toxicity.

Identified test compounds can be evaluated for toxicological effects using known methods (see, Lu, Basic Toxicology, Fundamentals, Target Organs, and Risk Assessment, Hemisphere Publishing Corp., Washington (1985); U.S. Pat. No.; 5,196,313 to Culbreth (issued Mar. 23, 1993) and U.S. Pat. No. 5,567,952 to Benet (issued Oct. 22, 1996)). For example, toxicology of a test compound can be established by determining in vitro toxicity towards a cell line, such as a mammalian, for example a human cell line. Test compounds can be treated with, for example, tissue extracts, such as preparations of liver, such as microsomal preparations, to determine increased or decreased toxicological properties of the test compound after being metabolized by a whole organism. The results of these types of studies are predictive of toxicological properties of chemical's in animals, such as mammals, including humans.

Alternatively, or in addition to these in vitro studies, the toxicological properties of a test compound in an animal model, such as mice, rats, rabbits, dogs or monkeys, can be determined using established methods (see, Lu, supra (1985); and Creasey, Drug Disposition in Humans, The Basis of Clinical Pharmacology, Oxford University Press, Oxford (1979)). Depending on the toxicity, target organ, tissue, locus and presumptive mechanism of the test compound, the skilled artisan would not be burdened to determine appropriate doses, LD₅₀ values, routes of administration and regimes that would be appropriate to determine the toxicological properties of the test compound. In addition to animal models, human clinical trials can be performed following established procedures, such as those set forth by the United States Food and Drug Administration (USFDA) or equivalents of other governments. These toxicity studies provide the basis for determining the efficacy of a test compound in vivo.

8. Efficacy of test compounds

Efficacy of a test compound can be established using several art recognized methods, such as in vitro methods, animal models or human clinical trials (see, Creasey, supra (1979)). Recognized in vitro models exist for several diseases or conditions. For example, the ability of a test compound to extend the life-span of HIV-infected cells in vitro is recognized as an acceptable model to identify chemicals expected to be efficacious to treat HIV infection or AIDS (see, Daluge et al., Antimicro. Agents Chemother. 41:1082-1093 (1995)). Furthermore, the ability of cyclosporin A (CsA) to prevent proliferation of T-cells in vitro has been established as an acceptable model to identify chemicals expected to be efficacious as immunosuppressants (see, Suthanthiran et al., supra (1996)). For nearly every class of therapeutic agent, disease or condition, an acceptable in vitro or animal model is available. The skilled artisan is armed with a wide variety of such models as they are available in the literature or from the USFDA or the National Institutes of Health (NIH). In addition, these in vitro methods can use tissue extracts, such as preparations of liver, such as microsomal preparations, to provide a reliable indication of the effects of metabolism on a test compound. Similarly, acceptable animal models can be used to establish efficacy of test compounds to treat various diseases or conditions. For example, the rabbit knee is an accepted model for testing agents for efficacy in treating arthritis (see, Shaw and Lacy, J. Bone Joint Surg. (Br.) 55:197-205 (1973)). Hydrocortisone, which is approved for use in humans to treat arthritis, is efficacious in this model which confirms the validity of this model (see, McDonough, Phys. Ther. 62:835-839 (1982)). When choosing an appropriate model to determine efficacy of test compounds, the skilled artisan can be guided by the state of the art, the USFDA or the NIH to choose an appropriate model, dose and route of administration, regime and endpoint and as such would not be unduly burdened.

In addition to animal models, human clinical trials can be used to determine the efficacy of test compounds. The USFDA, or equivalent governmental agencies, have established procedures for such studies.

9. Selectivity of test compounds

The in vitro and in vivo methods described above also establish the selectivity of a candidate modulator. It is recognized that chemicals can modulate a wide variety of biological processes or may be selective. Panels of cells as they are known in the art can be used to determine the specificity of the a test compound (WO 98/13353 to Whitney et al., published Apr. 2, 1998). Selectivity is evident, for example, in the field of chemotherapy, where the selectivity of a chemical to be toxic towards cancerous cells, but not towards non-cancerous cells, is obviously desirable. Selective modulators are preferable because they have fewer side effects in the clinical setting. The selectivity of a test compound can be established in vitro by testing the toxicity and effect of a test compound on a plurality of cell lines that exhibit a variety of cellular pathways and sensitivities. The data obtained form these in vitro toxicity studies can be extended to animal model studies, including human clinical trials, to determine toxicity, efficacy and selectivity of a test compound.

The selectivity, specificity and toxicology, as well as the general pharmacology, of a test compound can be often improved by generating additional test compounds based on the structure/property relationship of a test compound originally identified as having activity. Test compounds can be modified to improve various properties, such as affinity, life-time in blood, toxicology, specificity and membrane permeability. Such refined test compounds can be subjected to additional assays as they are known in the art or described herein. Methods for generating and analyzing such compounds or compositions are known in the art, such as U.S. Pat. No. 5,574,656 to Agrafiotis et al.

10. Pharmaceutical compositions

The present invention also encompasses a test compound in a pharmaceutical composition comprising a pharmaceutically acceptable carrier prepared for storage and preferably subsequent administration, which has a pharmaceutically effective amount of the test compound in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The test compounds of the present invention can be formulated and used as tablets, capsules or elixirs for oral administration; suppositories for rectal administration; sterile solutions or suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparations, such as liposomes, can be used.

The pharmaceutically effective amount of a test compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperiotoneally, employing a variety of dosage forms. Such methods can also be used in testing the activity of test compounds in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above, and can be guided by agencies such as the USFDA or NIH. Typically, human clinical applications of products are commenced at lower dosage levels, with dosage level being increased until the desired effect is achieved. Alternatively, acceptable in vitro studies can be used to establish useful doses and routes of administration of the test compounds.

In non-human animal studies, applications of the pharmaceutical compositions are commenced at higher dose levels, with the dosage being decreased until the desired effect is no longer achieved or adverse side effects are reduced of disappear. The dosage for the test compounds of the present invention can range broadly depending upon the desired affects, the therapeutic indication, route of administration and purity and activity of the test compound. Typically, dosages can be between about 1 ng/kg and about 10 mg/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ dysfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrans appropriate to the barrier to be permeated are used in the formulation. Such penetrans are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the test compounds of the invention to be formulated as tables, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries which facilitate processing of the active chemicals into preparations which can be used pharmaceutically. The preparations formulated for oral administration may be in the form of tables, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions.

Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The test compounds of the present invention, and pharmaceutical compositions that include such test compounds are useful for treating a variety of ailments in a patient, including a human. A patient in need of such treatment can be provided a test compound of the present invention, preferably in a pharmacological composition in an effective amount to reduce the symptoms, pathology or rate of progression of a disease or disorder in a patient. The amount, dosage, route of administration, regime and endpoint can all be determined using the procedures described herein or by appropriate government agencies, such as the United Stated Food and Drug Administration.

11. Treating Diabetes using Identified Compounds

Another aspect of the invention involves a method of treating diabetes by administering an effective amount of pharmaceutical composition of the present invention to a subject, such as a human patient, in need of treatment of diabetes. The pharmaceutical composition is administered to the subject in an amount, route of administration and regime sufficient to have a therapeutic, palliative, prophylactic, impeditive effect to ameliorate the effects, reversing the course of, delaying the onset of or preventing diabetes. The subject preferably is suspected of having or being at risk of developing diabetes.

An “effective amount” is the amount of a therapeutic reagent that when administered to a subject by an appropriate dose and regime produces the desired result.

A “subject in need of treatment for diabetes” is a subject diagnosed with diabetes or suspected of having diabetes.

A “therapeutic effect” is the reduction or elimination of a disease state or pathological condition.

A “palliative effect” is the alleviation of symptoms associated with a disease or pathological condition.

A “prophylactic effect” is the prevention of a disease state or pathological condition.

An “impeditive effect” is the reduction of the rate of progression of a disease state or pathological condition.

To “ameriolate the effects” refers to the reduction of the severity of the symptoms of a disease state or pathological condition.

To “reverse the course of diabetes disease” refers to the restoration or improvement of glucose metabolism in a subject.

1. Nucleic Acid Molecules

Therapeutic composition. The therapeutic composition of the present invention includes at least one nucleic acid molecule of the present invention, preferably a nucleic. The nucleic acids may be covalently or noncovalently conjugated or bound to other molecules, such as, but not limited to, proteins that may facilitate their delivery to the target tissue or tissues. Small molecules such as folate may be conjugated to nucleic acid molecules to enhance transport across the blood-brain barrier (Wu, D. et al. (1999) Pharm. Res. 16: 415-19.)

The nucleic acid molecules can be complexed with cationic lipids, packaged within liposomes, incorporated into hydrogels, cyclodextrins, biodegradable nanocapsules, or bioadhesive microspheres. The pharmaceutical compostition may include carriers, thickeners, diluents, buffers, preservatives, surface active agents, and the like in addition to oligonucleotides. Pharmaceutical compositions can also include one or more active ingredients such as antimicrobial agents, antiinflammatory agents, anesthetics, and the like in addition to oligonucleotides. If administration is by injection or infusion, the nucleic acid molecules cann be delivered directly or in the aforementioned compositions in sterile solution, which may also contain buffers, diluents, and other suitable additives. Formulations for topical administration may include ointments, lotions, creams, gels, drops, suppositories, sprays, liquids, and powders. Conventional pharmaceutical carriers, aqueous, powder or oily bases, thickeners and the like can be necessary or desirable.

Nasal inhalation may be particularly effective for delivery of pharmaceutical compositions to the brain (Wang, Y. et al. (1998) Biopharm Drug Dispos. 19: 571-5) and/or cerebrospinal fluid (Sakane T. (1991) J. Pharm. Pharmacol. 43: 449-51). Pharmaceutical compositions that include nucleic acid molecules can also include compounds that enhance absorption by nasal epithelial cells such as cationic compounds (Natsume, H. et al. (1999) Int. J Pharm. 185: 1-12), cyclodextrins (Martin, et al., J. Drug Target. 6: 17-36), or other compounds that are known or may be later discovered to enhance nasal absorption. Solutions containing nucleic acids for nasal delivery may be supplied in spray containers for aerosol inhalation.

Compositions for oral delivery include powders or granules, suspensions or solutions in water or nonaqueous media, capsules, sachets, or tablets. Thickeners, flavorings, diluents, emulsifiers, dispersing aids or binders may be desirable.

Dose. Optimum doses of pharmaceutical compositions that include nucleic acid molecules depends on a variety of factors, including the severity of the condition to be treated, the toxicity of the nucleic acid molecules being delivered, the route of administration, and the individual patients response to the treatment. The skilled practitioner is able to determine the appropriate dose based on these factors and the effective dose derived from animal and clinical studies. In general, dosage is from 0.01 micrograms to 100 g per kg of body weight, and may be given once or more daily, weekly, monthly, or yearly, or even once every 2 to 20 years. Persons of ordinary skill in the art can estimate repetition rates for dosing based on measured residence times and concentrations of the drug in bodily fluids or tissues. It may be desirable to have the patient undergo maintenance therapy to prevent the recurrence of the disease state, wherein the nucleic acids are administered in maintenance doses, ranging from 0.01 microgram to 100 g per kg of body weight once or more daily to once every 20 years.

Route of Administration. Nucleic acid molecules may be administered by any appropriate route of administration, such as, for example, parenteral or intravenous injection. Nucleic acids may also be delivered intravenously through pump, stent, or drip. Nucleic acid molecules may be introduced into the cerebrospinal fluid by injection into the spinal column. For delivery into the brain, injection may be into the brain cavity via a canula. Other routes of delivery include oral delivery and topical application. Nasal inhalation of aerosols may be particularly effective for administering the nucleic acids of the invention and their formulations to the brain. Nucleic acids may also be encased in or applied to a polymer, solid support or fabric, or gel which is delivered locally. Such solid supports, fabrics, polymers, or gels may be biodegradable.

Regime. The dose regime is determined experimentally based on animal studies and clinical trials. Doses may be given once or more daily, weekly, monthly, or yearly, or even once every 2 to 20 years. Persons of ordinary skill in the art can estimate repetition rates based on measured residence times and concentrations of the drug in bodily fluids or tissues. Following successful treatment, it may be desirable to have the patient under maintenance therapy to prevent the recurrence of the disease state, wherein the oligonucliotide is administered in maintenance doses, ranging from 0.01 micorgrams to 100 grams per kg of body weight, once or more daily, to once every 20 years.

Monitoring Progress. The progress of treatment for diabetes, either type I or type II, can be measured using methods known in the art. For example, blood glucose, urine glucose or blood or serum insulin levels can be monitored using established methods. These measurements can be taken at appropriate intervals, including before, during and after feeding or fasting. In this instance, the caloric intake and type of caloric intake, such as carbohydrates, should be noted.

2. Gene Therapy Constructs

Gene therapy constructs contain nucleic acids comprising a nucleic acid molecule of the present invention optionally operably linked to gene regulatory elements. The nucleic acid molecule and gene regulatory elements may be in a plasmid or may be incorporated into a vector, such as, but not limited to, a retroviral vector, an adenoviral vector, an adeno-associated viral vector, a vaccinia viral vector, a herpes viral vector, or other vectors as they are known or later developed in the art. The gene therapy constructs may be administered as DNA, as viral particles, or in cells.

Therapeutic composition. Gene therapy constructs that consist of nucleic acid molecules not incorporated into vectors such as viruses may be delivered as free nucleic acids, or may be delivered covalently or noncovalently conjugated or bound to other molecules, such as, but not limited to, molecules that enhance their transport across the blood-brain barrier or that may facilitate their delivery to the target tissue or tissues. Other DNA sequences, such as adenovirus VA genes can be included in the administration medium and be co-transfected with the gene of interest. The presence of genes coding for the adenovirus VA gene product may significantly enhance the translation of mRNA transcribed from the plasmid. Gene therapy constructs that are packaged in viruses may have proteins or other molecules or compounds, such as, but not limited to lipids, proteins, or polymers incorporated into or associated with the virus to enhance delivery into cells. The gene therapy constructs, whether naked DNA or packaged vector constructs, may be complexed with cationic lipids, packaged within liposomes, incorporated into hydrogels, cyclodextrins, biodegradable nanocapsules, or bioadhesive microspheres. The pharmaceutical composition may include carriers, thickeners, diluents, buffers, preservatives, surface active agents, and the like in addition to oligonucleotides. Pharmaceutical compositions may also include one or more active ingredients such as antimicrobial agents, antiinflammatory agents, anesthetics, and the like in addition to oligonucleotides. If administration is by injection or infusion, the gene therapy constructs may be delivered directly or in the aforementioned compositions in sterile solution, which may also contain buffers, diluents, and other suitable additives. Formulations for topical administration may include ointments, lotions, creams, gels, drops, suppositories, sprays, liquids, and powders. Conventional pharmaceutical carriers, aqueous, powder or oily bases, thickeners and the like may be necessary or desirable.

Nasal inhalation may be particularly effective for delivery of pharmaceutical compositions to the brain (Wang, Y. et al. (1998) Biopharm Drug Dispos. 19: 571-5) and/or cerebrospinal fluid (Sakane T. (1991) J. Pharm. PharmacoL 43: 449-51). Pharmaceutical compositions that include gene therapy constructs may also include compounds that enhance absorption by nasal epithelial cells such as cationic compounds (Natsume, H. et al. (1999) Int. J. Pharm. 185: 1-12), cyclodextrins (Martin, et al., J. Drug Target. 6: 17-36), or other compounds that are known or may be later discovered to enhance nasal absorption. Solutions containing gene therapy constructs may be supplied in spray containers for aerosol inhalation.

Compositions for oral delivery include powders or granules, suspensions or solutions in water or nonaqueous media, capsules, sachets, or tablets. Thickeners, flavorings, diluents, emulsifiers, dispersing aids or binders may be desirable. Nucleic acids may also be encased in or applied to a polymer, solid support or fabric, or gel which is delivered locally. Such solid supports, fabrics, polymers, or gels may be biodegradable.

Gene therapy constructs may also be delivered in cells. Cells containing gene therapy constructs may be derived from the patient, another human being, or even an animal of another species. Gene therapy constructs may be introduced into the cells ex vivo by viral transfection, electroporation, membrane fusion with liposomes, high velocity bombardment with DNA coated microprojectiles, incubation with calcium-phosphate-DNA precipitate, tansfection with DEAE-dextran, direct microinjection, or other methods known or later developed in the art. The cells are then delivered to the patient by any of a variety of means, including implantation or injection. The cells may express the gene therapy construct in vivo to obtain the therapeutic effect in the patient. Alternatively, after introduction into the patient, the cells containing the gene therapy construct may replicate and/or package the gene therapy construct such that endogenous cells in the patient may be infected, transformed, or transfected with the gene therapy construct and thereby express it. Cells containing gene therapy constructs may be enclosed in structures composed of polymers or other materials to retain them at the instillation site or to protect them from the patient's cellular immunity mechanisms.

Dose. Optimum doses depend on the severity of the condition to be treated, the toxicity of the gene therapy construct being delivered, the route of administration, and the individual patient's response to the treatment. The skilled practitioner is able to determine the appropriate dose based on these factors and the effective dose derived from animal and clinical studies. In general, for naked DNA gene therapy constructs, the dosage is from 0.01 micrograms to 100 g per kg of body weight. For viral gene therapy constructs, an appropriate dose is in the range of 0.1 to 50 ml of 10⁶ to 10¹¹ particle forming units per ml viral expression vectors.. For cells containing viral expression constructs, about 10⁵ to about 10⁸ cells may be delivered to an appropriate site.

Route of Administration. Naked DNA gene therapy constructs and viral gene therapy constructs may be delivered by intravenous or intraperitoneal injection, intratracheally, intrathecally parenterally, intraarticularly, intramuscularly, or introduced into the brain by injection via a cannula or injected into the spinal column for distribution within the cerebrospinal fluid. Gene therapy constructs may be administered intravenously, by injection, catheter, pump, or drip. Alternatively, Cells containing gene therapy constructs may be implanted surgically into the brain, or they may be delivered to another site in the body. This may be convenient if the protein or nucleic acid molecules expressed from the gene therapy construct is targeted to the brain or, if the cells are packaging cells, the virus produced by the introduced cells may be targeted to the brain or other relevant tissue. Cells may be administered topically, intraocularly, parenterally, intranasally, intratracheally, intrabronchially, intramuscularly, subcutaneously, or by any other means.

Regime. The dose regime is determined experimentally based on animal studies and clinical trials. Doses may be given once or more daily, weekly, monthly, or yearly, or even once every 2 to 20 years. Persons of ordinary skill in the art can estimate repetition rates based on measured residence times and concentrations of the gene product of the gene therapy vector in bodily fluids or tissues. Following successful treatment, it may be desirable to have the patient receive additional doses of the gene therapy vector if it is determined that levels of the gene product have declined below a level necessary to prevent disease progression, or if there are symptoms of disease progression. The gene therapy construct or cells containing the gene therapy construct may be administered in maintenance doses, where the dose has been determined based on animal and clinical studies, and may be monitored by measuring the expression product of the gene therapy construct in the patient's bodily fluids.

Monitoring Progress. The progress of treatment for diabetes, either type I or type II, can be measured using methods known in the art. For example, blood glucose, urine glucose or blood or serum insulin levels can be monitored using established methods. These measurements can be taken at appropriate intervals, including before, during and after feeding or fasting. In this instance, the caloric intake and type of caloric intake, such as carbohydrates, should be noted.

3. Biologically Active Agents.

Therapeutic composition. A therapeutic composition of the present invention can include at least one biologically active agent of the present invention. At least one biologically active agent of the present invention can optionally be covalently or noncovalently conjugated or bound to other molecules, such as, but not limited to, proteins that may facilitate their delivery to the target tissue or tissues. Small molecules such as folate may be conjugated to the biologically active agents of the invention to enhance transport across the blood-brain barrier (Wu, D. et al. (1999) Pharm. Res. 16: 415-19.). The pharmaceutical composition may comprise a pharmaceutically acceptable carrier prepared for storage and preferably subsequent administration, which has a pharmaceutically effective amount of the biologically active agent in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The biologically active agents of the present invention can be formulated and used as tablets, capsules or elixirs for oral administration; suppositories for rectal administration; sterile solutions or suspensions for injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparations, such as liposomes, can be used. The pharmaceutical composition may also include carriers, thickeners, diluents, buffers, preservatives, surface active agents, and the like in addition to one or more biologically active agents. Pharmaceutical compositions may also include one or more active ingredients such as antimicrobial agents, antiinflammatory agents, anesthetics, and the like in addition to the biologically active agents of the invention. Formulations for topical administration may include ointments, lotions, creams, gels, drops, suppositories, sprays, liquids, and powders. Conventional pharmaceutical carriers, aqueous, powder or oily bases, thickeners and the like may be necessary or desirable.

Agents intended to be administered intracellularly may be administered using techniques well known to those of ordinary skill in the art. For example, such agents may be encapsulated into liposomes, then administered as described above. Substantially all organic molecules present in an aqueous solution at the time of liposome formation are incorporated into or within the liposomes thus formed. The liposomal contents are both protected from the external micro-environment and, because liposomes fuse with cell membranes, are efficiently delivered into the cell cytoplasm. Additionally, due to their hydrophobicity, small organic molecules can be directly administered intracellularly.

Nasal inhalation may be particularly effective for delivery of pharmaceutical compositions to the brain (Wang, Y. et al. (1998) Biopharm Drug Dispos. 19: 571-5) and/or cerebrospinal fluid (Sakane T. (1991) J. Pharm. Pharmacol. 43: 449-51). Pharmaceutical compositions that include biologically active agents may also include compounds that enhance absorption by nasal epithelial cells such as cationic compounds (Natsume, H. et al. (1999) Int. J. Pharm. 185: 1-12), cyclodextrins (Martin, et al., J. Drug Target. 6: 17-36), or other compounds that are known or may be later discovered to enhance nasal absorption. Solutions containing biologically active agents for nasal delivery may be supplied in spray containers for aerosol inhalation.

Dose. The pharmaceutically effective amount of a biologically active agent of the present invention required as a dose will depend on the route of administration and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. The skilled practitioner is able to determine the appropriate dose based on these factors and the effective dose derived from animal and clinical studies.. The determination of effective dosage levels, that is the dose levels necessary to achieve the desired result, can be accomplished by one skilled in the art using routine methods. Typically, human clinical applications of products are commenced at lower dosage levels, with dosage level being increased until the desired effect is achieved. Alternatively, acceptable in vitro studies can be used to establish useful doses and routes of administration of the bioactive compounds and bioactivities.

Route of Administration. In employing them in vivo, the pharmaceutical compositions containing at least one biologically active agent of the present invention can be administered to the patient in a variety of ways, including, for example, parenterally, intravenously, subcutaneously, intramuscularly, colonically, rectally, nasally or intraperiotoneally, employing a variety of dosage forms. Biologically active agents may be introduced into the cerebrospinal fluid by injection into the spinal column. For delivery into the brain, injection may be into the brain via cannula. Other routes of delivery include oral delivery and topical application. Nasal inhalation of aerosols may be particularly effective for administering the biologically active agents of the invention and their formulations to the brain.

Regime. It will be recognized by one of skill in the art that the optimal quantity and spacing of individual dosages of a biologically active agent of the present invention will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of biologically active agent of the invention given per day for a defined number of days, can be ascertained by those skilled in the art using conventional course of treatment determination tests. Persons of ordinary skill in the art can estimate repetition rates based on measured residence times and concentrations of the biologically active agent in bodily fluids or tissues. Following successful treatment, it may be desirable to have the patient receive maintenance doses of the biologically active agent, where the maintenance dose has been determined based on animal and clinical studies..

Monitoring Progress. The progress of treatment for diabetes, either type I or type II, can be measured using methods known in the art. For example, blood glucose, urine glucose or blood or serum insulin levels can be monitored using established methods. These measurements can be taken at appropriate intervals, including before, during and after feeding or fasting. In this instance, the caloric intake and type of caloric intake, such as carbohydrates, should be noted.

### EXAMPLES

The following examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

### Example 1

### Glucose Respondiveness is Linked to Mitochondrial DNA Content

In order to determine if a correlation exists between mitochondrial mass and/or function, the following experiments were carried out.

Generation of INS-1 Cells Depleted of Mitochondrial DNA

INS-1 rat insulinoma cells were provided by Prof. Claes Wollheim, University Medical Centre, Geneva, Switzerland, and cultured at 37° C. in a humidified 5% CO₂ environment in RPMI cell culture media (Gibco BRL, Gaithersburg, Md.) supplemented with 10% fetal bovine serum (Irvine Scientific), 2 mM L-glutamine, 100 U/ml penicillin, 100 μg/ml streptomycin, 10 mM HEPES, 1 mM sodium pyruvate and 50 μM β-mercaptoethanol.

INS-1 cells were cultured for 3-60 days under conditions as described above except media were additionally supplemented with 50 μg/ml uridine and nucleoside analogs 2′3′-dideoxycytidine [ddC], 2′3′-dideoxyinosine [ddl] or 2′3′-didehydro-3-deoxythymidine [d4T] (all from Sigma) at varying concentrations (1-500 μM) diluted from 100× stock in PBS or a comparable dilution of PBS without. Media were replenished every two days. Cells were harvested at periodic intervals and assayed for insulin secretion and mtDNA content.

Total DNA was prepared from rat liver (for probing rat-derived cells) or the murine cell line 3T3 L1 (for probing mouse-derived cells; see Green et al., Cell 3:127-133, 1974 and Cell 5:19-27, 1975) using DNAzol™ reagents (Molecular Research Center, Inc., Cincinnati, Ohio) and method essentially according to the manufacturer's instructions. The template DNAs were examined by agarose gel electrophoresis and ethidium bromide staining and found to be roughly equivalent. Each template DNA was used in separate polymerase chain reaction (PCR) reactions to prepare DNA molecules having 1,207 base pairs and corresponding to either nucleotides 5342 to 6549 of the rat (Rattus norvegicus) mitochondrial genome (GenBank Accession No. X14848, Anderson et al., Nature 290:497-516, 1981) or nucleotides 5361 to 6568 of the murine (Mus musculus) mitochondrial genome (GenBank Accession No. V00711, Bibb et al., Cell 26:167-180, 1981). The same pair of oligonucleotide primers, specific for the mitochondrially encoded cytochrome c oxidase subunit I (COX-I) gene, were used for reactions for either rat or mouse templates. The pair of primers consisted of forward and reverse oligonucleotides having the following sequences:

| | | |
|---|---|---|
| Forward: | 5′-CACAAAGATATCGGAACCCTCTA | (SEQ ID NO:_) |
| Reverse: | 5′-AAGTGGGCTTTTGCTCATGTGTCAT | (SEQ ID NO:_) |

The PCR reactions contained appropriate amounts of template DNA, primers, MgCl₂, all four dNTPs, reaction buffer, and Taq polymerase, brought up to a volume of 50 μl using sterile water. The reactions were incubated at 95° C. for 10 seconds, followed by 30 cycles of 95° C. for 1 minute, 60° C. for 1 minute and 72° C. for 1 minute, after which the reactions were incubated at 72° C. for 4 minutes and then cooled to 4° C.

The PCR reactions mixes were extracted with phenol:chloroform and, along with a series of molecular weight markers, electrophoresed on an agarose gel that was stained with ethidium bromide and visualized with ultraviolet light. For both reactions, a single band of the predicted size (i.e., about 1.2 kilobases) was observed. The rat probe was radiolabeled with ³²P using a Prime-a-Gene® random priming kit (Promega, Madison, Wis.) essentially according to the manufacturer's instructions.

To quantify mitochondrial DNA by slot blotting, INS-1 cells, or ρ⁰ INS-1 cells generated using ddC as described above, were seeded into 12-well plates containing RPMI media supplemented as described above at 0.4×10 cells/well and cultured at 37° C., 5% CO₂ for 2 days. Cells (0.7×10 cells/well) were rinsed with PBS and total cellular DNA was extracted using DNAzol (Molecular Research Center, Inc., Cincinnati, Ohio) according to the manufacturer's instructions. One hundred ng DNA from each cell preparation was slot-blotted onto a Zeta-Probe membrane (Bio-Rad, Hercules, Calif.) and crosslinked at 125 joules using a BioRad GS GeneLinker irradiation/energy source.

The membranes were rinsed in hybridization buffer (5×SSC, 0.1% N-laurylsarcosine, 0.02% SDS, 1% blocking solution, Boehringer Mannheim, Indianapolis, Ind.) and hybridized overnight in the same buffer at 42° C. with the [³²P]-labeled rat COX I probe. Following hybridization, membranes were washed twice with 2×SSC/0.1% SDS and twice with 0.1×SSC/0.1% SDS and exposed to X-ray film. Mitochondrial DNA was quantified by densitometric scanning of the resulting autoradiographs.

Incubation of INS-1 cells with ddC, ddl or d4T for seven days decreased mtDNA content in a dose-dependent fashion. The relative mtDNA content (mean COX-I hybridization signal+SEM) of the cells, normalized to total cellular DNA, is plotted as a function of nucleoside analog concentration in FIG. 1A. The IC₅₀ for ddC was approximately 50 μM. In INS-1 cells incubated with 25 μM ddC for up to 40 days, the decline in mtDNA content was time-dependent, with a t_{½} of approximately three days; mtDNA was undetectable in these cells after 21 days.

Glucose-Responsive Insulin Production by INS-1 Cells Depleted of Mitochondrial DNA

INS-1 cells, or ρ⁰ INS-1 cells generated using ddC as described above, were seeded into 12-well plates containing RPMI media supplemented as described at 0.5×10⁶ cells/well and cultured at 37° C., 5% CO₂ for 2 days. Cells (0.7×10⁶ cells/well were rinsed with glucose-free KRH buffer (134 mM NaCl, 4.7 mM KCI, 1.2 mM KH₂PO₄, 1.2 mM MgSO₄, 1.0 mM CaCl₂, 10 mM HEPES, 10 mM NaHCO₃, 0.5% BSA), then incubated in the same buffer for 1 hr at 37° C. in a humidified 5% CO₂/95% air atmosphere. Fresh KRH buffer containing 0.5 mM isobutylmethyl xanthine and the following secretagogues was added: 5 mM glucose, 10 mM glucose, 20 mM glucose, 5 mM KCl or 20 mM KCl. After an additional 1 hr at 37° C., 5% CO₂ the culture supernatants were collected. Insulin concentrations in the supernatants were measured and normalized to cell number using an insulin-specific radioimmunoassay kit (ICN Biochemicals, Irvine, Calif.) according to the manufacturer's instructions.

As expected, untreated (mitochondrially proficient) INS-1 cells begin to exhibit glucose-mediated insulin secretion at concentrations of glucose starting at 5 mM (FIG. 1B, “parental INS-1”). In contrast, in cells treated with ddC (10 μM) for over 20 days, at which time mtDNA was significantly reduced, glucose stimulated insulin secretion was not observed at any glucose level tested (FIG. 1B, “mtDNA-depleted INS-1”).

Other Glucose-Mediated Responses are Blunted in INS-1 Cells Depleted of Mitochondrial DNA

The ability of mitochondrially proficient and INS-1 cells that have been treated with ddC, and thus depleted of mtDNA, to respond to glucose in other ways was examined.

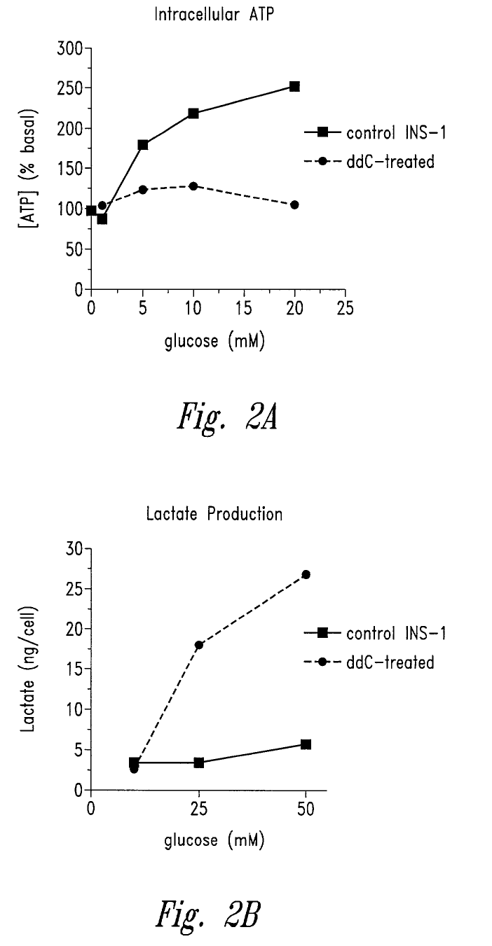

Intracellular ATP levels were determined using an ATP bioluminescent assay kit (Sigma) for both types of cells in response to various doses of glucose. The results (FIG. 2A) show that untreated INS-1 cells produce increasing amounts of ATP in response to increasing amounts of glucose. In contrast, INS-1 cells that have been substantially depleted of mtDNA, although able to maintain a basal level of ATP, do not show any substantial response to stimulation by glucose.

Lactate production was also determined for both types of cells in response to various doses of glucose. Cells were grown in 35 mm dishes with various concentrations of glucose. Media were replenished about 16 hr before assay with normal culture media containing various amounts of glucose. The media were then collected, and lactate measured using a commercially available kit, in which lactate dehydrogenase is used to produce a fluorescent compound (Sigma, St. Louis, Mo.), essentially according to the manufacturer's instructions.

The results (FIG. 2B) show that untreated INS-1 cells maintain abasal level of lactate and produce only slightly increasing amounts of ATP in response to increasing amounts of glucose. In contrast, INS-1 cells that have been substantially depleted of mtDNA show any substantial response to stimulation by glucose.

These results indicate that, at a minimum, functioning mitochondria promote glucose-responsiveness in insulin-secreting cells, and suggest that functioning mitochondria are required for a robust production of insulin, ATP and lactate in response to glucose in such cells.

### Example 2

### Preparation of NRF-1 Expression Constructs

This Example describes the preparation of a variety of expression constructs that are designed to overpress the human NRF1 protein or fusion protein derivatives thereof. Although the gene (nrf-1) encoding human NRF1 is used in this Example, nucleotide sequences of nrf-1 genes form other species are known and may be employed in like fashion.

A. PCR Amplification of Human NRF1 cDNAs

A CDNA library derived from total cellular RNA prepared from human placenta was obtained from a commercial source (Clontech, Palo Alto, Calif.). The RNA was purified by treatment with RNase-free DNase I (Roche Molecular Biochemicals, formerly Boehringer Mannheim Biochemicals, Indianapolis, Ind.) using 1 μl of DNase I (10 U/μl ) in a buffer containing 40 mM Trsi-HCl, pH 7.0, 6 mM magnesium chloride and 2 mM calcium chloride for 30 minutes at 37° C. This treatment was followed by two phenol/chloroform extractions, one chloroform extraction and an ethanol precipitation in the presence of sodium acetate. The RNA pellet was collected by centrifugation, washed with 70% ethanol, air dried, and resuspended in RNase-free sterile water. The RNA was reverse transcribed to generate cDNA using RNase H-deficient Reverse Transcriptase (SUPERSCRIPT™; Life Technologies, Rockville, Md.).

Human NRF1 cDNAs were amplified by polymerase chain reactions (PCR) in a thermal cycler using the following primers, AMPLITAQ™ DNA Polymerase (Perkin-Elmer, Inc., Norwalk, Conn.), and reagents and buffers supplied in a GENEAMP™ PCR Reagent Kit (Perkin-Elmer), according to the manufacturer's instructions. In the following representations of the PCR primers, underlined nucleotides indicate sequences complementary to the 5′-ends and 3′-ends of the human NRF1 cDNAs, double-underlined nucleotides indicate recognition sequences for the restriction enzymes BamHI (recognition sequence: 5′-GGATCC) and Asp718 (recognition sequence: 5′-GGTACC), and the huNRF1 start codon (ATG) and the reverse complement of the stop codon (TGA, having the reverse complement TCA) are emboldened.

For human NRF1 (huNRF1; SEQ ID NO:______), primers having the following nucleotide sequence were used:

| | |
|---|---|
| Forward (sense): | |
| 5′-TATAAAGGATCCATGGAGGAACACGGAGTGACC, and | SEQ ID NO:_{—} |
| Reverse (antisense): | |
| 5′-AATTTAGGTACCTCACTGTTCCAATGTCACCACC | SEQ ID NO:_{—.} |

The PCR products were digested with BamHI and Asp718 (both enzymes from Roche Molecular Biochemicals) essentially according to the manufacturer's recommendations using manufacturer-supplied reaction buffers. The restriction enzyme digested DNAs were purified by horizontal agarose gel electrophoresis and band extraction using the UltraClean™ GelSpin kit (Mo Bio Laboratories, Inc., Solana Beach, Calif.).

B. Generation of a Yeast huNRF1 Expression Construct

A yeast huNRF 1 expression vector was constructed using the expression vector pYPGE2, which comprises a TRP1 selectable marker and the strong PGK promoter upstream from a multiple cloning site (Brunelli and Pall, 1993 Yeast 9:1299-1308). Plasmid pYPGE2 DNA was digested with BamHI and Asp718, gel-purified and ligated with the BamHI- and Asp718-digested huNRF1 PCR product of the preceding section. The ligation mixture was used to transform competent E. coli cells. Plasmid DNA was isolated from several independently isolated bacterial cultures using the WIZARD™ Plus Series 9600 Miniprep Reagents System (Promega, Madison, Wis.) and were restriction mapped to confirm the structure of the expected expression construct. One confirmed plasmid was chosen to be used for further study and was designated “pPGK.hNRF1.” The nucleotide sequence of the huNRF-1-encoding DNA inserted into pPGK.hNRF1 was determined according to standard methods known in the art in order to confirm its veracity.

C. Generation of a huNRF1 Expression Construct

The expression vector pcDNA3.1 (Invitrogen, Carlsbad, Calif.) was used. This vector contains the following elements operably linked in a 5′ to 3′ orientation: the cytomegalovirus (CMV) enhancer/promoter (P_{CMV}); a multiple cloning site (MCS) containing recognition sequences for several restriction enzymes; and the bovine growth hormone (BGH) polyadenylation signal and transcription termination sequence to enhance mRNA stability. The expression vector also contains an ampicillin resistance gene for positive selection of transformants in prokaryotes, e.g., E. coli, as well as a neomycin resistance gene for positive selection of transformants in mammalian cells, and origins of replication for bacterial and mammalian cells (ColEl- and SV40-derived, respectively). The SV40 origin of replication allows for episomal replication of the expression construct as well as simple vector rescue in cells expressing the large T antigen of SV40 (i.e., COS-1 or COS-7 cells, ATCC accession numbers CRL-1650 and CRL-1651, respectively).

Plasmid pcDNA3.1 (“-” version) was prepared by digestion with the restriction endonucleases BamHI and Asp718 essentially according to the manufacturer's (Roche) instructions, and subjected to horizontal agarose gel electrophoresis and band extraction using the UltraClean™ GelSpin kit (Mo Bio Laboratories). Plasmid pPGK.hNRF1 (see preceding section) was digested with BamHI and Asp718, subjected to horizontal agarose gel electrophoresis, and the restriction fragment comprising the huNRF 1 sequences was extracted from the gel using the UltraClean™ GelSpin kit. The extracted huNRF1 DNA was ligated into the similarly-digested pcDNA3 expression vector DNA using T4 DNA ligase (New England Biolabs, Beverly, Mass.) using the manufacturer's reaction buffer and following the manufacturer's instructions. Competent E. coli cells (strain DH5α; Life Technologies, Inc. {Gibco BRL}, Gaithersburg, Md.) were transformed with ligation mixtures according to the manufacturer's instructions. Single colonies were selected and grown in 3-5 ml of LB broth (Sambrook, J., Fritsch, E. F., and Maniatis, T., Molecular Cloning: A Laboratory Manual, Cold Spring Harbor Laboratory Press, Cold Spring Harbor, N.Y., 1989) containing 50 μg/ml ampicillin (Roche Molecular Biochemicals). Plasmid DNA was isolated from the bacterial cultures using the WIZARD™ Plus Series 9600 Miniprep Reagents System (Promega). A few isolates of pcDNA3.1(−)-derived huNRF1 expression constructs were restriction mapped to confirm their structures. One isolate of a pcDNA3.1-huNRF1 expression construct having the predicted restriction map was selected for further experiments and designated “pCDNA3.1.huNRF1.” The nucleotide sequence of the huNRF-1-encoding DNA inserted into pCDNA3.1.huNRF1 was determined according to standard methods known in the art in order to confirm its veracity.

D. Generation of Epitope-Tagged, Tightly Regulated huNRF 1 Fusion Protein Expression Constructs

Human NRF1 cDNA was amplified from pCDNA3.1.huNRF1 by polymerase chain reactions (PCR) in a thermal cycler using the following primers, AMPLITAQ™ DNA Polymerase (Perkin-Elmer), and reagents and buffers supplied in a GENEAMP™ PCR Reagent Kit (Perkin-Elmer), according to the manufacturer's instructions. In the following representations of the PCR primers, underlined nucleotides indicate sequences complementary to the 5′-ends and 3′-ends of the human NRF 1 cDNAs, double-underlined nucleotides indicate recognition sequences for the restriction enzymes BamHI (recognition sequence: 5′-GGATCC) and Xhol (recognition sequence: 5′-CTCGAG), the huNRF1 start codon (ATG) and the reverse complement of the stop codon (TGA, having the reverse complement TCA) are emboldened.

| | |
|---|---|
| Forward-Flag(−) (5′→3′): | |
| TATGGATCCATGGAGGAACACGGAGTGACC, | SEQ ID NO:_{—} |
| Forward-Flag(=) (5′→3′): | |
| TATGGATCCATGGAGTACAAGGACGATGACAAGATGGAGGAACACGGA, and | SEQ ID NO:_{—} |
| Reverse (5′→3′): | |
| AATCTCGAGTCACTGTTCCAATGTCACCACC | SEQ ID NO:_{—} |

The same reverse primer was used in each set of PCR reactions, but two different forward primers, with or without sequences encoding an epitope tag, were used. The nucleotide sequence in the Forward-Flag(+) primer that is flanked by the BamHI recognition site on its 5′ end, and by the huNRF-1 start codon on its 3′ end, encodes an epitope known as FLAG® (see Hopp, Biotechnology 6:1204-1210, 1988). The PCR products were digested with the restriction endonucleases BamHI and Xhol essentially according to the manufacturer's (Roche) instructions, and subjected to horizontal agarose gel electrophoresis and band extraction using the UltraClean™ GelSpin kit (Mo Bio Laboratories).

The expression vector pcDNA4.TO (Invitrogen) was used. This vector contains the following elements operably linked in a 5′ to 3′ orientation: the cytomegalovirus (CMV) enhancer/promoter (P_{CMV}); two copies of the tetracycline operator 2 (TetO₂) site; a multiple cloning site (MCS) containing recognition sequences for several restriction enzymes; and the bovine growth hormone (BGH) polyadenylation signal and transcription termination sequence to enhance mRNA stability. The expression vector also contains an ampicillin resistance gene for positive selection of transformants in prokaryotes, e.g., E. coli, as well as a Zeocin™ resistance gene for positive selection of transformants in mammalian cells, and origins of replication for bacterial and mammalian cells, the latter of which is SV40-derived. The SV40 origin of replication allows for episomal replication of the expression construct as well as simple vector rescue in cells expressing the large T antigen of SV40 (i.e., COS-1 or COS-7 cells, ATCC accession numbers CRL-1650 and CRL-1651, respectively).

Plasmid pcDNA4.TO (“-” version) was prepared by digestion with the restriction endonucleases BamHI and XhoI essentially according to the manufacturer's (Roche) instructions, and subjected to horizontal agarose gel electrophoresis and band extraction using the UltraClean™ GelSpin kit (Mo Bio Laboratories). The extracted huNRF1 PCR products were separately ligated into the similarly-digested pcDNA4.TO expression vector DNA using T4 DNA ligase (New England Biolabs) using the manufacturer's reaction buffer and following the manufacturer's instructions. Competent E. coli cells were transformed with ligation mixture, and single colonies were selected and grown in 3-5 ml of LB broth containing 50 μg/ml ampicillin (Roche). Plasmid DNA was isolated from the bacterial cultures using the WIZARD™ Plus Series 9600 Miniprep Reagents System (Promega). A few candidate isolates of “pcDNA4/TO.hNRF1” and “pcDNA4/TO.FLAG.hNRF1” expression constructs were restriction mapped to confirm their structures. One isolate of each expression construct having the predicted restriction map was selected for further experiments.

### Example 3

### Expression of Human NRF 1 in Transfected Cells

The pcDNA4/TO-derived expression construct pcDNA4/TO.FLAG.hNRF1 was transfected into HEK293 cells (ATCC No. CRL-1573) that have been genetically engineered to stably express the tetracycline repressor protein (TetR; see, e.g., Yao et al., 1998 Hum. Gene Therapy 9:1939; Yao et al., 1999 Hum. Gene Therapy 10:419) (T-Rex-293™ cell line; Invitrogen) using the FUGENE™ transfection reagent (Roche Molecular Biochemicals, Indianapolis, Ind.). The T-Rex-293™ cells and tranformants thereof were cultured and maintained essentially according to the manufacturer's instructions.

Stable clones of T-Rex-293™ cells transfected with pcDNA4/TO.huNRF1 were treated with 1 μg/ml tetracycline. Tetracycline binds to tetracycline repressor (TetR) molecules in the cell, causing them to be released from the 2×TetO2 regulatory region, thereby allowing transcription of the nrf-1 gene to proceed, with the predicted result being that such treatment will result in overexpression of NRF-1 in transfected cells as compared to non-transfected cells or to cells transfected with vector (pcDNA4/TO) DNA that lacks any NRF-1-encoding sequences.

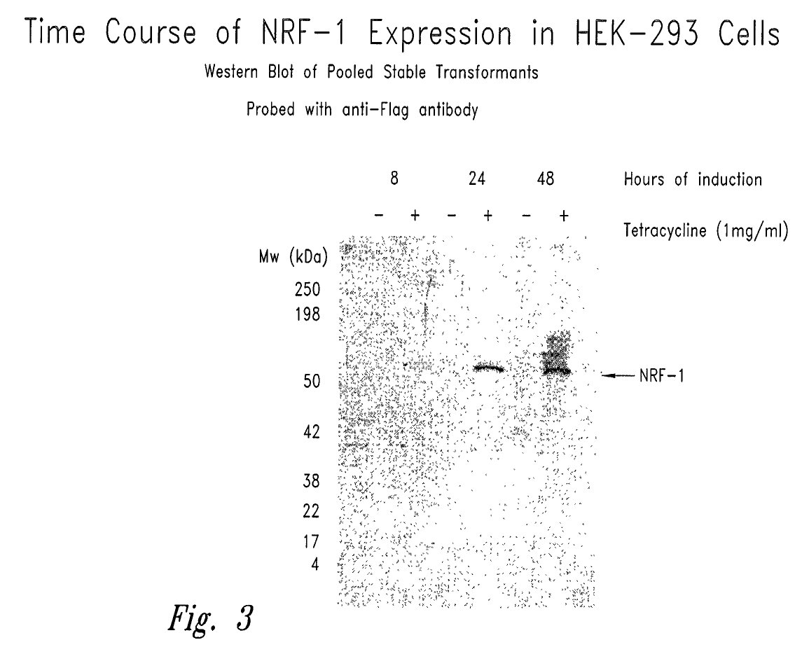

The cells were pooled and harvested at different time points (i.e., 0, 8, 24, and 48 hours), lysed and used to prepare protein extracts that were subjected to Western blot analysis using an anti-FLAG® antibody (Zymed Laboratories, Inc., South San Francisco, Calif.). In non-transfected cells FLAG®-tagged huNRF-1 was not detected, whereas cells transfected with pcDNA4/TO.hNRF1 show a low level of anti-FLAG® reactive material 8 hours after the initiation of tetracylcine induction, and increasing amounts of the FLAG®t-tagged huNRF1 protein was detected over time. The amount of FLAG®-reacting material further increased after 24 hours, with the highest detection observed after 48 hours in the presence of tetracycline (FIG. 3). A FITC conjugate of anti-FLAG® (Zymed) was used to visually examine cells harboring pcDNA4/TO.hNRF1 after induction using fluorescent microscopy. The results demonstrate that, as expected, the FLAG-huNRF 1 fusion protein localizes to the nuclei of cells.

### Example 4

### Assays in Cells Overexpressing NRF1

Effects of expression and overexpression of huNRFI in cell lines of interest for screening and other assays relating to specific mitochondrial diseases are evaluated as follows. Appropriate cell lines such as INS-1 insulinoma cells for a diabetes model system, or SH-SY5Y neutorblastoma cells and cybrids derived therefrom for Alzheimer's or Parkinson's disease models, are used. These cell lines are first genetically engineered to stably express the tetracycline repressor protein (TetR, see e.g., Yao et al., 1998 Hum. Gene Therapy 9:1939; Yao et al., 1999 Hum. Gene Therapy 10:419) using methods known in the art (e.g., pcDNA6/TR, available from Invitrogen, Carlsbad, Calif.) and as described in the preceding example. The TetR-expressing cells are then transformed with expression constructs derived from pcDNA4/TO (e.g., pcDNA4/TO.FLAG.hNRF1, pcDNA4/TO.hNRF1) according to standard methods known in the art. Stable clones, and optimal times of induction and concentrations of tetracycline, are determined as in the preceding sections.

After induction of stable clones for different periods of time, and/or different concentrations of tetracyline, the impact of expression and overexpression of huNRF-1 on mitochondrial biogenesis and function is determined by examining one or more indicators of mitochondrial function. The mitochondrial mass of cells overexpressing huNRF1 is determined by methods known in the art and compared to the mitochondrial mass of non-transfected cells. The impact of NRF1 overexpression on transcription of other nuclear mitochondrial genes such as cytochrome C (CytC), the transcription factor mtTFA, MRP, r12s, and ATP6 genes is examined. After induction for different periods of time, and/or different concentrations of tetracyline, RNA is extracted from the cells using standard methods (Sambrook et al,. A Laboratory Manual, Cold Spring Harbor Laboratory Press, 1989). Quantitative PCR is used to determine levels of mRNA transcripts of nuclear-encoded mitochondrial genes. In a similar fashion, Western blots are used to measure the affect on the translation products of the mitochondrial genes in NRF-1 overexpressing cells. Also microscopy, such as electron microscopy, is employed to directly assess mitochondrial parameters, e.g., volume, mass and the like.

Indicators of mitochondrial function are examined before and after induction of huNRF1 in cells having impaired mitochondrial function (e.g., cybrid cells comprising mitochondria derived from an individual known to be diabetic, or INS-1 cells that have been substantially depleted of mtDNA), in order to determine the extent to which NRF1 restores mitochondrial function. In the case of glucose-responsive cells having impaired mitochondrial function (e.g., INS-1 cells that have been substantially depleted of mtDNA), the extent to NRF1 restores glucose responsiveness is determined as in the preceding Examples. In the case of glucose-responsive cells that do not have impaired mitochondrial function (e.g., INS-1 cells), the extent to which expression of NRF-1 enhances the rapidity, extent and/or duration of cellular responses to glucose, as determined by measuring, e.g., insulin secretion, ATP synthesis or lactate production, is determined as in Example 1.

One or more indicators of mitochondrial ftmction or, in appropriate cell types, glucose responsiveness, that are increased after induction of NRF1 are used as the standards by which the effects of candidate compounds or compositions are evaluted in assays for identifying compounds or compositions useful for treating mitochondrial diseases. For example, in the case of diabetes, INS-1 cells are contacted with one or more candidate compounds or compositions and evaluated with regard to at least one indicator of mitochondrial function and/or at least one indicator of glucose responsiveness. Compounds and compositions useful for treating diabetes are identified as those candidate compounds or compositions that result in a change in an indicator of mitochondrial function and/or a change in an indicator of glucose responsiveness, wherein said change(s) parallels, is similar to, or exceeds the change(s) in these indicators that result from NRF1 expression or overexpression.

### Example 5

### PGC-1 Overexpression in Transfected Cells

Tightly-regulated (e.g. tetracyline-inducible) expression constructs expressing PGC-1 are prepared as in the preceding Examples. Nucleotide sequences encoding PGC-1 are known and are used to design PCR primers for the amplification of pgc-1 cDNAs (Puigserver et al., 1998 Cell 92:829; Wu et al., 1999 Cell 98:115). Cells that have been genetically engineered to stably express the tetracycline repressor protein (TetR, see e.g., Yao et al., 1998 Hum. Gene Therapy 9:1939; Yao et al., 1999 Hum. Gene Therapy 10:419) using methods known in the art (e.g., pcDNA6/TR, available from Invitrogen, Carlsbad, Calif.) are transfected with these expression constructs, and are used (1) to examine the impact of PGC-1 expression and overexpression on mitochondrial biogenesis and function, and (2) in assays for identifying compounds and/or compositions useful for treating mitochondrial diseases, as in the preceding Examples.

Cells that overexpress NRF1 and PGC-1 are prepared as follows. Appropraite cell lines are co-transfected with an NRF1 expression construct and with a PGC-1 expression construct. Alternatively, tightly-regulated (e.g., tetracyline-inducible) expression constructs capable of expressing both NRF1 and PGC-1 are prepared as in the preceding Examples.

Cells expressing TetR are transfected with these expression constructs, and are used (1) to examine the impact of the combination of NRF1 and PGC-1 expression and overexpression on mitochondrial biogenesis and function, and (2) in assays for identifying compounds and/or compositions useful for treating mitochondrial diseases, as in the preceding Examples.

### Example 6

### NRF-1 Overexpression

SH-SY5Y neuroblastoma cells (ATCC, Manassas, Va.) cell lines were genetically engineered to stably express the tetracycline repressor protein (TetR, see e.g., Yao et al., 1998 Hum. Gene Therapy 9:1939; Yao et al., 1999 Hum. Gene Therapy 10:419) using methods known in the art (e.g., pcDNA6/TR, available from Invitrogen, Carlsbad, Calif.) and were then transfected with the tightly regulated, tetracycline inducible NRF-1 construct described above (e.g., Example 2). These cells were capable of overexpressing NRF-1 following 48h induction (Ind) with 10 ng/ml of tetracycline, (Calbiochem, San Diego, Calif.). Tetracycline-induced, NRF-1 overexpressing SY5Y cells, and control (Con), non-induced SH-SY5Y cells, were labeled with 250 μCi ³⁵S-Express (methionine/cysteine) (New England Nuclear, Boston, Mass.) for 1 h at 37° C. in 5% CO₂ incubator. Following rinses in media and PBS, cells were scraped in 1 ml PBS containing 1 mM phenylmethylsulfonylfluoride (PMSF; all chemicals from Sigma, St. Louis, Mo., unless otherwise indicated) and pelleted at low speed in Eppendorf™ microcentrifuge tubes. Cell pellets were solubilized in lysis buffer (50 mM Tris pH 7.4, 1% Nonidet-P40™, 2.5% NaDeoxycholate, 150 mM NaCl, 1 mM EDTA, 1 mM PMSF, 1 mM aprotinin, 1 mg/ml leupeptin, 1 mg/ml pepstatin, 1 mM NaVO₃ and 1 mM NaF) on ice for 15 minutes, spun at 20,000×g in a refrigerated centrifuge for 20 minutes, and supernatant protein concentrations were determined using the BCA protein assay (Pierce Chemicals, Rockford, Ill.).

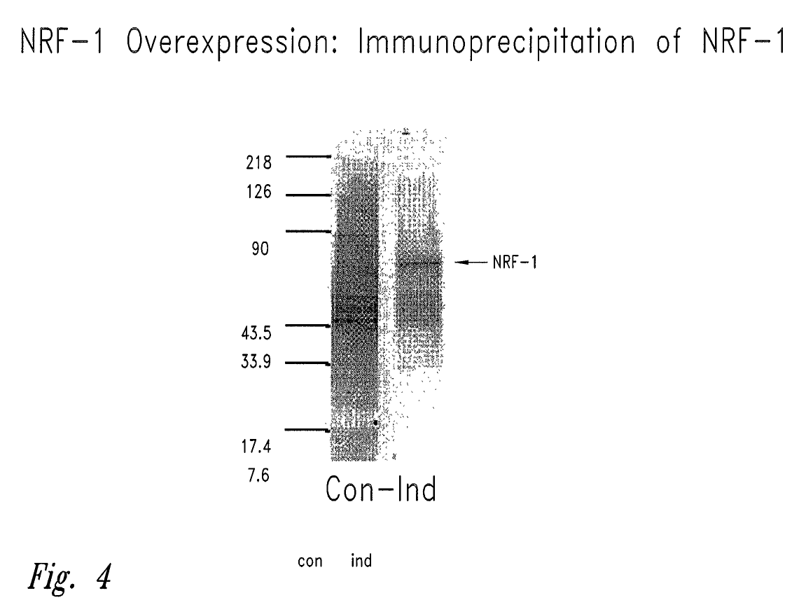

Equal amounts of protein for control, non-induced (Con) and tetracycline-induced (Ind) samples were each brought to a total volume of 500 μl with cell lysis buffer and the anti-NRF-1 polyclonal antibody (Gugneja et al., 1997 J Biol. Chem. 272:18732) was added at a 1:500 dilution. Lysates were incubated by rotating at 4° C. overnight. Protein A (Calbiochem, La Jolla, Calif.) was added to the immunoprecipitation reaction mixtures according to the supplier's recommendations, reactions were allowed to continue rotating at 4° C. for 1 h and were then pelleted by centrifugation. Each pellet was washed 3× with ice cold PBS+PMSF, brought up in 2× Laemmli's sample buffer, boiled for 5 min and electrophoresed on a 12% polyacrylamide gel using SDS-Tris-Glycine running buffer according to standard procedures. The gel was fixed, treated with Entensify™ autoradiography/fluoragrophy reagent (New England Nuclear), dried and then exposed to autoradiography film (Xomat™, Kodak, Rochester, N.Y.) for 5 days. As shown in FIG. 4, a specific band for overexpressed, transfected NRF-1 was apparent only in the immunoprecipitates from tetracycline induced cells.

TABLE 2
NRF-1 Overexpressing mRNA Clone 3

| GENE | % Increase | % Decrease |
|---|---|---|
| NRF-1 | 800 | |
| ND1 | 92 | |
| ATP6 | 85 | |
| MtTFA | | 46 |
| CytoC | 28 | |
| ALA-S | 144 | |

Table 2 shows results from a representative experiment in which mRNA was quantified from Tet^{r}-transfected, SH-SY5Y NRF-1 cells overexpressing transfected NRF-1 following 48h induction with 50 ng/ml of tetracyline (Calbiochem, La Jolla, Calif.); values in Table 2 are expressed as percentage of increased or decreased mRNA detected for the indicated gene transcript in the tetracycline-induced cells, relative to the corresponding mRNA level in non-induced control cells. RNA was collected from cells using the Trizol™ reagent (Life Technologies, Inc., Bethesda, Md.) method according to the supplier's instructions, and was then treated with DNAse (Promega, Madison, Wis.) to remove any contaminating DNA for 30 minutes at 37° C., followed by one phenol/chloroform/isoamyl, alcohol extraction and a precipitation with LiC1 (Sigma) in ethanol. The RNA pellet was then washed two times in 70% ethanol, resuspended in diethylpyrocarbonate- (DEPC) (Ambion, Austin, Tex.) treated water, quantitated with RiboGreen™ indicator reagent according to the manufacturer's recommendations (Molecular Probes, Eugene, Oreg.) and then used in an RT-PCR reaction using oligo dT primers (Superscript First-Strand Synthesis System for RT-PCR, Life Technologies, Inc.) to generate cDNA. Serial dilutions of the cDNA were used in real-time PCR in the ABI Prism 7700™ Sequence Detection System (Perkin-Elmer, Inc., ABI Division, Foster City, Calif.) to determine gene expression in the induced cells relative to the control cells. Genes of interest were probed, along with β-actin as a normalizer, using primers and probes (GenSet Corp., La Jolla, Calif.) generated from specific sequences for the genes of interest as follows:

| | | | |
|---|---|---|---|
| NRF-1 | FORWARD: | CACTTACTGGAGTCCAAGATGCTAAT | [SEQ ID NO:14] |
| | REVERSE: | TGGTGACTGCGCTGTCTGATAT | [SEQ ID NO:15] |
| | PROBE: | CCTGGTCCAGATCCCGTGAGCATGTAC | [SEQ ID NO:16] |
| ND1 | FORWARD: | CCTCCCTGTTCTTATGAATTCGA | [SEQ ID NO:17] |
| | REVERSE: | TTTTTTCATAGGAGGTGTATGTATGAGTTG | [SEQ ID NO:18] |
| | PROBE: | AGCATACCCCCGATTCCGCTACGA | [SEQ ID NO:19] |
| ATP6 | FORWARD: | CGCCACCCTAGCAATATCA | [SEQ ID NO:20] |
| | REVERSE: | CGACAGCGATTTCTAGGATAGTCA | [SEQ ID NO:21] |
| | PROBE: | CCATTAACCTTCCCTCTACACTTATCATCTTCACAATTC | [SEQ ID NO:22] |
| MRP | FORWARD: | GAGAGTGCCACGTGCATACG | [SEQ ID NO:23] |
| | REVERSE: | ACGCTTCTTGGCGGACTTT | [SEQ ID NO:24] |
| | PROBE: | ACGTAGACATTCCCCGCTTCCCACTC | [SEQ ID NO:25] |
| mtTFA | FORWARD: | TGATCCAGAAAGAAAACTTGTATTATGTG | [SEQ ID NO:26] |
| | REVERSE: | AAACAGGCTTTTATACGTTATGCAAA | [SEQ ID NO:27] |
| | PROBE: | AGAAATCTAAAAAACGAAAAGTCTCCAAAGTCTCTGGAA | [SEQ ID NO:28] |
| cytoC | FORWARD: | CATTCAGAAACAAACTGTAGAACTGTGTA | [SEQ ID NO:29] |
| | REVERSE: | GTGTATATCTCCGTTACTTTAATCCTTTTAAG | [SEQ ID NO:30] |
| | PROBE: | TTGATTGGGAATGGTGCTTTTGCCA | [SEQ ID NO:31] |
| ALA-S | FORWARD: | TTCACTTAACCCCAGGCCATT | [SEQ ID NO:32] |
| | REVERSE: | AATTTATTTCCAGGACTATGTTTTTACTATAGATT | [SEQ ID NO:33] |
| | PROBE: | TCATATCCAGATGGTCTTCAGTTGTCTTTATATGTG | [SEQ ID NO:34] |
| βActin | FORWARD: | CTGGAACGGTGAAGGTGACA | [SEQ ID NO:35] |
| | REVERSE: | CGGCCACATTGTGAACTTTG | [SEQ ID NO:36] |
| | PROBE: | CAGTCGGTTGGAGCGAGCATCCC | [SEQ ID NO:37] |

The following formula was used for quantitation of mRNA:

$$\% Change of normalized induced gene from normalized non \text{-} induced gene = \frac{\begin{matrix}[ induced gene / induced actin ] - \\ [ non \text{-} induced gene / non \text{-} induced actin ]\end{matrix}}{[ non \text{-} induced gene / non \text{-} induced actin ]} \times 100$$

A 100% increase indicates a 2 fold change in mRNA. As seen in Table 2, NRF-1 was greatly overexpressed in induced SH-SY5Y transfectants, as also was δ-aminolevulinate synthase (ALA-S, FIG. 5), consistent with observations in a different transfected cell line, HeLa cells (Li et al., 1999 J. Biol. Chem. 274:17534). Unexpectedly, increases in the mitochondrial gene transcripts for ND1 (mitochondrial NADH dehydrogenase) and ATP6 (mitochondrial ATP synthase subunit 6) genes were also observed, with these mRNAs being expressed at levels approximately 2-fold greater in induced cells than in the control cells. There did not appear to be an increase in either cytochrome c or mtTFA message levels in the induced cells. The results suggested that NRF-1 overexpression alone was able to influence transcription levels of mRNAs corresponding to several target genes that have been implicated in mitochondrial biogenesis.

### Example 7

### Altered Gene Expression in NRF- 1 Overexpressing Cells

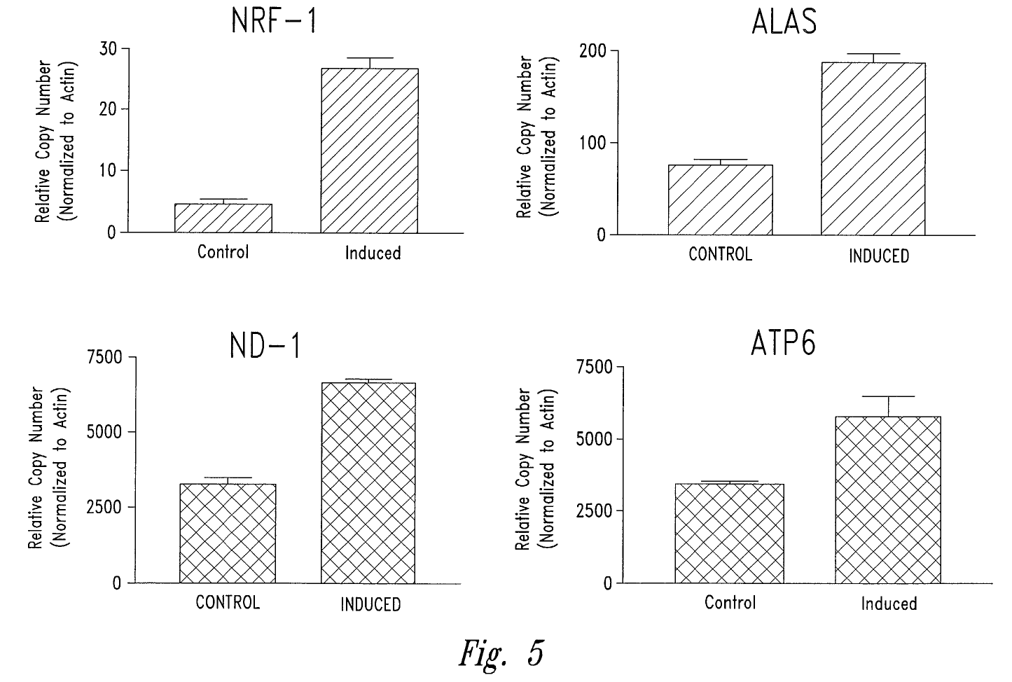

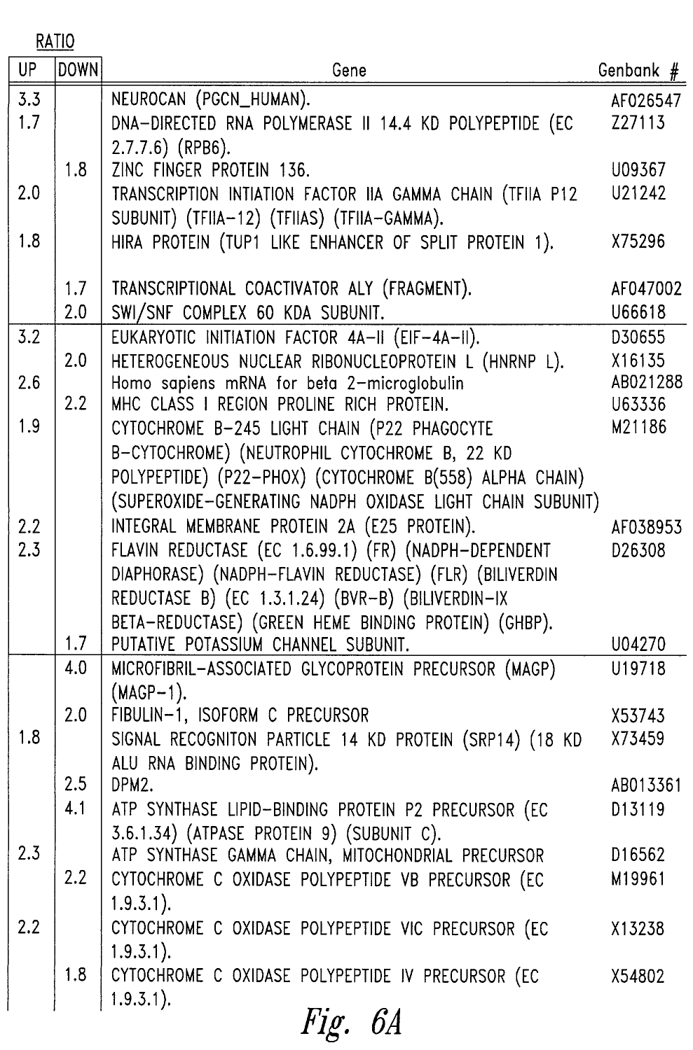

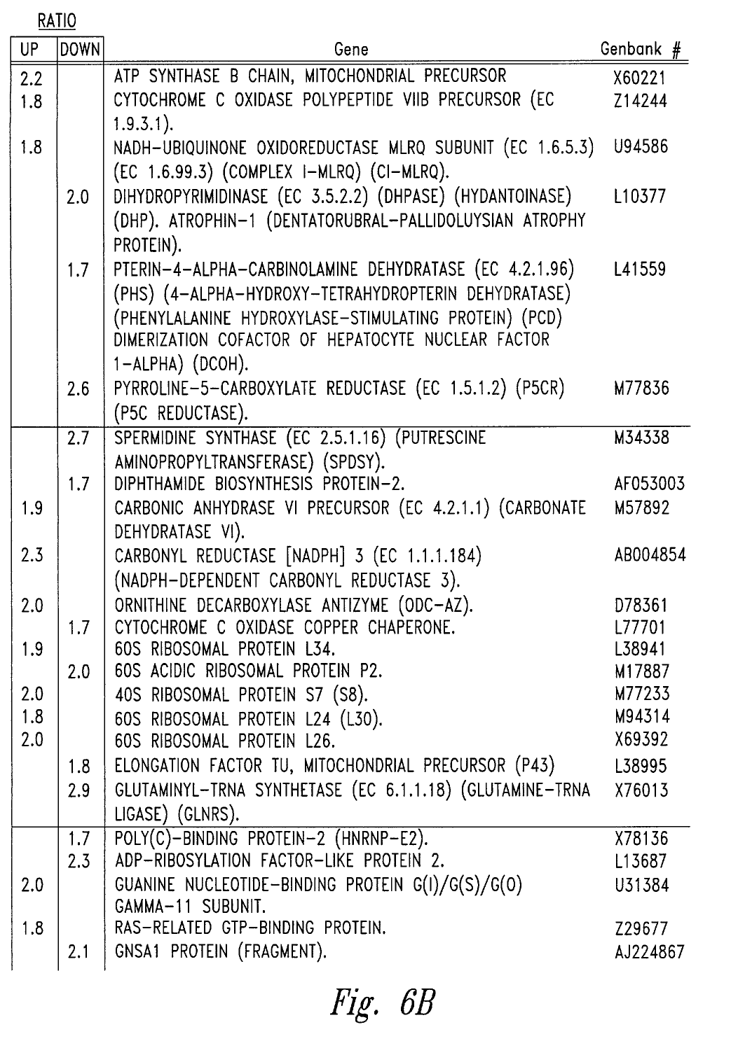

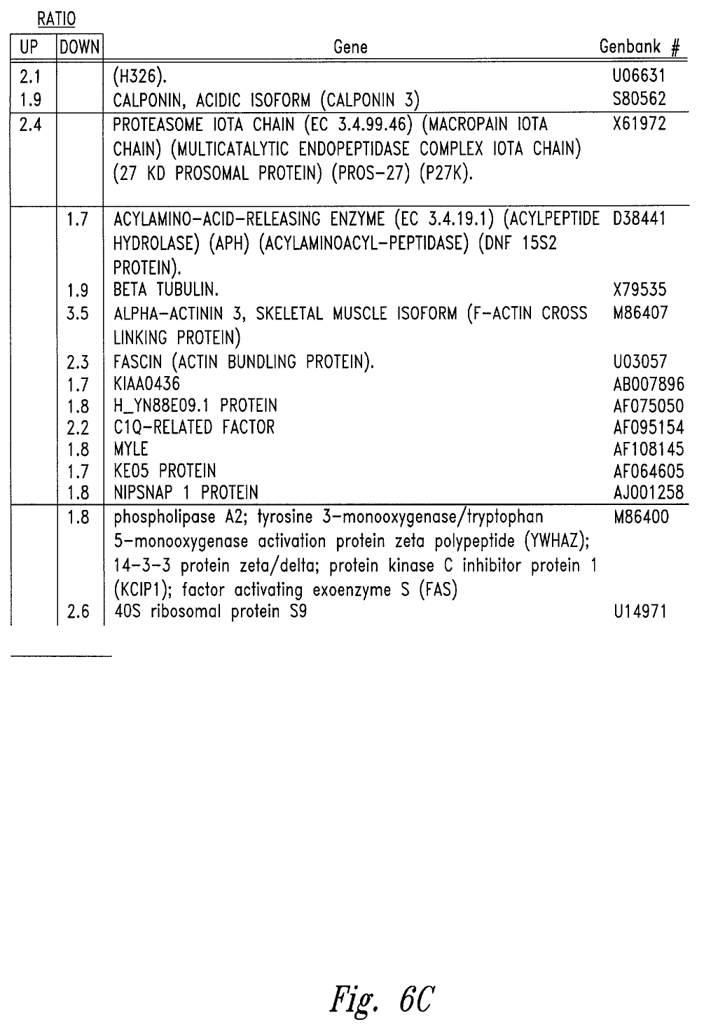

Total RNA was extracted from tetracycline-induced and control, uninduced Tet^{R} SH-SY5Y NRF-1 transfectants as described in the preceding Example, radiolabeled and hybridized to nylon membranes on which were immobilized an array of 1056 isolated human cDNA sequences (Atlas™ human 1.2k-III array, lot no. 0030342, Clontech, Palo Alto, Calif.) using standard methodologies and according to the supplier's recommendations. Comparison of induced and uninduced RNA samples for hybridization signal intensities demonstrated significant alteration of the mRNA levels of over 60 genes (FIG. 6). Genes exhibiting altered expression included ATP synthase gamma, cytochrome c oxidase Vb, cytochrome c oxidase VIc, transcription factor II gamma, eukaryotic initiation factor 4A-II, ATP synthase B, ATP synthase C, cytochrome c oxidase VIb and ETC Complex I MLRQ, many of which are associated with mitochondrial functions.

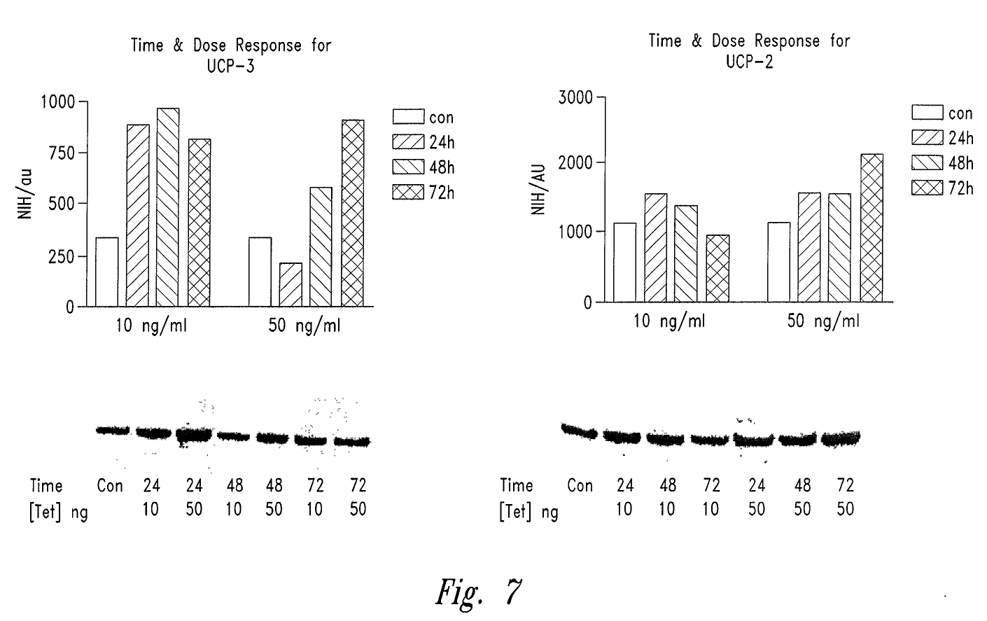

The effect of induced NRF-1 overexpression was also investigated at the level of expressed protein products by western immunoblot analysis according to established procedures. Briefly, tetracycline induced (10 or 50 ng/ml for 24, 48 or 72 hours as indicated in FIG. 7) or control, uninduced SH-SY5Y NRF-1 transfectants were lysed in cell lysis buffer (50 mM Tris-HCl, pH 7.4; 1% Nonidet™ P-40 (NP-40), 0.25% sodium deoxycholate; 150 mM NaCl; 1 mM EDTA; 1 mM PMSF; 1 μg/ml each of aprotinin, leupeptin and pepstatin), protein content of lysates determined using the BCA microwell plate protocol (Pierce Chemicals, Inc., Rockford, Ill.) according to the supplier's instructions, and equal protein levels of the total cell lysates were electrophoresed by SDS-polyacrylamide gel electrophoresis. Resolved proteins were then transferred to Immobilin-P™ (Millipore, Bedford, Mass.) membranes. The membranes were washed, probed with rabbit polyclonal primary antibodies (Alpha Diagnostic, Inc., San Antonio, Tex.) specific for human mitochondrial Uncoupling Protein-2 (UCP-2) and Uncoupling Protein-3 (UCP-3), developed with horseradish peroxidase-conjugated anti-rabbit Ig antibodies and the ECL™ chemiluminescent detection reagent (both from Amersham Pharmacia Biotech, Piscataway, N.J.) according to the suppliers' recommendations, and autoradiographed according to standard methodologies. Analysis of the resulting autoradiograms to determine the extent of increased protein levels in the NRF-1 overexpressing cells relative to the control, non-induced cells was conducted using the publicly available National Institutes of Health NIH-Image analysis software package (http://rsb.info.nih.gov/nih-image/). Results are presented in FIG. 7, including relative quantification of UCP-3 and UCP-2 signals according to arbitrary absorbance units (AU; i.e., optical density) of each gel lane in the autoradiograph.

All publications, including patent documents and scientific articles, referred to in this application are incorporated by reference in their entireties for all purposes to the same extent as if each individual publication were individually incorporated by reference. All headings are for the convenience of the reader and should not be used to limit the meaning of the text that follows the heading, unless so specified.

From the foregoing it will be appreciated that, although specific embodiments of the invention have been described herein for purposes of illustration, various modifications may be made without deviating from the spirit and scope of the invention. Accordingly, the invention is not limited except as by the appended claims

What is claimed is:

1. A method for treating a disease associated with altered mitochondrial function comprising administering an agent that increases mitochondrial mass in cells in an individual in need thereof.

2. The method of claim 1 wherein the disease associated with altered mitochondrial function is diabetes.

3. The method of claim 2 wherein the diabetes is type 2 diabetes mellitus.

4. The method of claim 1 wherein the agent that increases mitochondrial mass induces expression of a gene selected from the group consisting of a PGC gene and a NRF gene.

5. The method of claim 4 where the PGC gene is PGC-1.

6. The method of claim 4 wherein the agent that increases mitochondrial mass is provided in a pharmaceutical composition.

7. The method of claim 4 wherein the NRF gene is NRF-1.

8. A method for treating a disease associated with altered mitochondrial function comprising administering an agent that alters mitochondrial function in cells in an individual in need thereof.

9. The method of claim 8 wherein the disease associated with altered mitochondrial function is diabetes.

10. The method of claim 9 wherein the diabetes is type 2 diabetes mellitus.

11. The method of claim 8 wherein the mitochondrial function is selected from the group consisting of oxygen consumption, mitochondrial biogenesis, oxidative phosphorylation, glucose-stimulated insulin secretion and apoptosis.

12. The method of either claim 1 or claim 8 wherein the cells are pancreatic cells.

13. The method of claim 12 wherein the pancreatic cells are pancreatic beta cells.

14. The method of either claim 1 or claim 8 wherein the cells are treated with at least one agent selected from the group consisting of an agent that alters the expression of a PGC gene, an agent that alters the expression of a NRF gene, an agent that alters the activity of a PGC gene product and an agent that alters the activity of a NRF gene product.

15. The method of claim 14 wherein the agent is selected from the group consisting of a polypeptide, a nucleic acid, a small molecule, a gene therapy construct and a test compound.

16. The method of claim 14 wherein the PGC gene is PGC-1.

17. The method of claim 14 wherein the agent i s provided in a pharmaceutical composition.

18. The method of claim 14 wherein the NRF gene is NRF- 1.

19. A method for identifying an agent for treating a disease associated with altered mitochondrial function, comprising contacting a cell comprising a regulatory expression construct with at least one candidate agent, wherein the regulatory expression construct comprises at least one regulatory element that is derived from a gene selected from the group consisting of a PGC gene and an NRF gene and that is operably linked to a reporter gene, and wherein the candidate agent alters the expression of the reporter gene relative to reporter gene expression in the absence of the candidate agent, and therefrom identifying an agent for treating the disease associated with altered mitochondrial funtion.

20. A method for identifying an agent for treating a disease associated with altered mitochondrial function, comprising contacting a candidate agent with a sample comprising a mitochondrion, wherein the mitochondrion comprises an expression construct encoding one or more proteins selected from the group consisting of an NRF protein and a PGC protein; and determining a level of at least one indicator of mitochondrial function, wherein the candidate agent alters the level of said indicator of mitochondrial function relative to the level of said indicator in the absence of the agent, and therefrom identifying an agent for treating a disease associated with altered mitochondrial function.

21. A method for identifying an agent for treating a disease associated with altered mitochondrial function, comprising contacting a candidate agent with a sample comprising a mitochondrion, wherein the mitochondrion comprises a product of an expression construct encoding one or more proteins selected from the group consisting of an NRF protein and a PGC protein; and determining a level of at least one indicator of mitochondrial function, wherein the candidate agent alters the level of said indicator of mitochondrial function relative to the level of said indicator in the absence of the agent, and therefrom identifying an agent for treating a disease associated with altered mitochondrial function.

22. A method for identifying an agent for treating a disease associated with altered mitochondrial function, comprising contacting a candidate agent with a sample comprising a cell containing a mitochondrion, wherein the cell comprises an expression construct encoding one or more proteins selected from the group consisting of an NRF protein and a PGC protein; and determining a level of at least one indicator of mitochondrial function, wherein the candidate agent alters the level of said indicator of mitochondrial function relative to the level of said indicator in the absence of the agent, and therefrom identifying an agent for treating a disease associated with altered mitochondrial function.

23. A method for identifying an agent for treating a disease associated with altered mitochondrial function, comprising contacting a candidate agent with a sample comprising a cell containing a mitochondrion, wherein the cell comprises a product of an expression construct encoding one or more proteins selected from the group consisting of an NRF protein and a PGC protein; and determining a level of at least one indicator of mitochondrial function, wherein the candidate agent alters the level of said indicator of mitochondrial function relative to the level of said indicator in the absence of the agent, and therefrom identifying an agent for treating a disease associated with altered mitochondrial function.

24. The method of any one of claims 19-23 wherein the disease associated with altered mitochondrial function is diabetes.

25. The method of any one of claims 20-23 wherein the indicator of mitochondrial function is glucose responsiveness.

26. A method for identifying a regulator of mitochondrial biogenesis, comprising contacting a stimulus with a cell comprising a mitochondrion under conditions and for a time sufficient to induce mitochondrial biogenesis; and detecting an altered level of a candidate signaling molecule, wherein an altered level of the candidate signaling molecule in a cell that has been contacted with the stimulus that induces mitochondrial biogenesis relative to the level of the candidate signaling molecule in a cell that has not been contacted with the stimulus indicates that the candidate signaling molecule is a regulator of mitochondrial biogenesis.

27. The method of claim 26 wherein the stimulus is selected from the group consisting of cold stress, an electrical stimulus and an adrenergic stimulus.

28. The method of claim 26 wherein mitochondrial biogenesis is detected by determining an indicator of mitochondrial function selected from the group consisting of oxygen consumption, amount of mitochondrial DNA, mitochondrial mass and an ATP biosynthesis factor.

29. The method of claim 26 wherein the candidate signaling molecule regulates activity of a gene selected from the group consisting of a PGC gene and a NRF gene.

30. The method of claim 26 wherein the candidate signaling molecule is regulated by a gene selected from the group consisting of a PGC gene and a NRF gene.

31. The method of claim 26 wherein the altered level of the candidate signaling molecule is a level selected from the group consisting of a level of a nucleic acid, a level of a polypeptide and a level of phosphorylation of a protein.

32. A method for identifying an agent that alters activity of a regulator of mitochondrial biogenesis for treating a disease associated with altered mitochondrial function, comprising contacting, in the presence of a candidate agent, a stimulus with a cell comprising a mitochondrion under conditions and for a time sufficient to induce an altered level of a signaling molecule that regulates mitochondrial biogenesis, wherein an altered level of the signaling molecule that regulates mitochondrial biogenesis in a cell that has been contacted with the candidate agent relative to the level of the signaling molecule that regulates mitochondrial biogenesis in a cell that has not been contacted with the candidate agent indicates that the agent alters activity of a regulator of mitochondrial biogenesis.

33. A method of identifying a gene encoding a target for therapeutic intervention in a disease associated with altered mitochondrial function, comprising:
(a) comparing (i) a first plurality of isolated nucleic acid molecules derived from a first biological source in which expression of a gene known to alter mitochondrial biogenesis has been induced, to (ii) a second plurality of isolated nucleic acid molecules derived from a second biological source in which expression of the gene known to alter mitochondrial biogenesis has not been induced, wherein the presence of at least one differentially expressed nucleic acid molecule in (i) or (ii) indicates the differentially expressed nucleic acid molecule is a candidate gene encoding a target for therapeutic intervention in a disease associated with altered mitochondrial function; and
(b) determining that altered expression of said candidate gene alters mitochondrial biogenesis, and therefrom identifying a gene encoding a target for therapeutic intervention in a disease associated with altered mitochondrial function.

34. The method of claim 33 wherein the gene known to alter mitochondrial biogenesis is selected from the group consisting of a PGC gene and a NRF gene.

35. The method of claim 33 wherein mitochondrial biogenesis is determined by measuring oxygen consumption.

36. The method of claim 33 wherein mitochondrial biogenesis is determined by detecting an indicator of mitochondrial function selected from the group consisting of oxygen consumption, amount of mitochondrial DNA, mitochondrial mass and an ATP biosynthesis factor.

37. The method of claim 33 wherein altered expression of the candidate gene is increased expression.

38. The method of claim 33 wherein altered expression of the candidate gene is decreased expression.
